(19) United States
(12) Patent Application Publication
(10) Pub. No.: US 2025/0017992 A1
(43) Pub. Date: Jan. 16, 2025

(54) COMPOSITIONS AND METHODS FOR TREATING OVARIAN AGING, OVARIAN DISEASES AND CONDITIONS, AND SYMPTOMS THEREOF

(71) Applicant: YUVA BIOSCIENCES, INC., Birmingham, AL (US)

(72) Inventor: Keshav K. Singh, Birmingham, AL (US)

(21) Appl. No.: 18/690,132

(22) PCT Filed: Sep. 7, 2022
(86) PCT No.: PCT/US2022/042799

§ 371 Date: Mar. 7, 2024

Related U.S. Application Data
(60) Provisional application No. 63/241,429, filed on Sep. 7, 2021.

(51) Int. Cl. A61K 36/03; A61K 36/47; A61K 45/06

(52) U.S. Cl. CPC A61K 36/03; A61K 36/47; A61K 45/06

(57) ABSTRACT

Methods of enhancing or improving fertility are disclosed. Methods of extending reproductive longevity are disclosed. Methods of increasing oocyte mitochondrial mass or preventing a decrease of oocyte mitochondrial mass are disclosed. Methods of improving embryo development or improving embryo fertilization rate are disclosed. Methods of treating or preventing an ovarian disease or condition are disclosed. Methods of reducing, preventing, or delaying perimenopause, menopause, or a symptom thereof are disclosed. The methods include administering an Emblica extract or a compound constituent of or having a similarity score of at least 95% with a compound constituent of an Emblica extract, or Fucus extract or a compound constituent of or having a similarity score of at least 95% with a compound constituent of a Fucus extract, or a chebula extract or a compound constituent of or having a similarity score of at least 95% with a compound constituent of a chebula extract.

### CROSS-REFERENCE TO RELATED APPLICATIONS

This application claims priority to U.S. Provisional Application Ser. No. 63/241,429, filed on Sep. 7, 2021, titled “COMPOSITIONS AND METHODS FOR TREATING OVARIAN DISEASES AND CONDITIONS AND SYMPTOMS THEREOF.” which is incorporated herein by reference in its entirety for all purposes.

### FIELD OF TECHNOLOGY

Aspects and embodiments disclosed herein relate to methods of treatment of diseases and conditions related to mitochondrial dysfunction. More specifically, aspects and embodiments disclosed herein relate to methods of treatment of diseases and conditions related to mitochondrial DNA depletion.

### BACKGROUND

Mitochondrial dysfunction, such as mitochondrial DNA (mtDNA) depletion, is involved in many diseases and conditions, such as mtDNA depletion syndromes, mitochondrial diseases, viral infections, viral infection-induced symptoms, aging, aging-associated chronic diseases, reduced energy levels and vitality, and other human pathologies. Fundamental questions about mitochondrial biology and mtDNA biology and their roles in such diseases and conditions remain mostly unsolved. Animal models capable of inducing mitochondrial dysfunction and/or modulating mtDNA copy number and/or concentration have been developed and provide a system for investigating mitochondrial pathology and its role in the disease process. There is a need for treatment methods for diseases and conditions related to mitochondrial dysfunction, such as mtDNA depletion.

### SUMMARY

In accordance with one aspect, there is provided a method of enhancing or improving fertility in a subject. The method may comprise administering to the subject a composition comprising an effective amount of one or more of an Emblica extract, a Fucus extract, and a chebula extract, or a pharmaceutically acceptable form thereof, or one or more compounds constituent of one or more of an Emblica extract and a Fucus extract, and a chebula extract, or a pharmaceutically acceptable form thereof, or one or more compounds having a similarity score of at least 95% with a compound constituent of one or more of an Emblica extract, a Fucus extract, and a chebula extract, or a pharmaceutically acceptable form thereof.

In accordance with one aspect, there is provided a method of extending reproductive longevity in a subject. The method may comprise administering to the subject a composition comprising an effective amount of one or more of an Emblica extract, a Fucus extract, and a chebula extract, or a pharmaceutically acceptable form thereof, or one or more compounds constituent of one or more of an Emblica extract, a Fucus extract, and a chebula extract, or a pharmaceutically acceptable form thereof, or one or more compounds having a similarity score of at least 95% with a compound constituent of one or more of an Emblica extract, a Fucus extract, and a chebula extract, or a pharmaceutically acceptable form thereof.

In accordance with one aspect, there is provided a method of increasing oocyte mitochondrial mass or preventing a decrease of oocyte mitochondrial mass in a subject. The method may comprise administering to the subject a composition comprising an effective amount of one or more of an Emblica extract, a Fucus extract, and a chebula extract, or a pharmaceutically acceptable form thereof, or one or more compounds constituent of one or more of an Emblica extract, a Fucus extract, and a chebula extract, or a pharmaceutically acceptable form thereof, or one or more compounds having a similarity score of at least 95% with a compound constituent of one or more of an Emblica extract, a Fucus extract, and a chebula extract, or a pharmaceutically acceptable form thereof.

In accordance with one aspect, there is provided a method of improving embryo development or improving embryo fertilization rate. The method may comprise administering to a subject source of the embryo or to the embryo a composition comprising an effective amount of one or more of an Emblica extract, a Fucus extract, and a chebula extract, or a pharmaceutically acceptable form thereof, or one or more compounds constituent of one or more of an Emblica extract, a Fucus extract, and a chebula extract, or a pharmaceutically acceptable form thereof, or one or more compounds having a similarity score of at least 95% with a compound constituent of one or more of an Emblica extract, a Fucus extract, and a chebula extract, or a pharmaceutically acceptable form thereof.

In accordance with one aspect, there is provided a method of treating or preventing an ovarian disease or condition in a subject. The method may comprise administering to the subject a composition comprising an effective amount of one or more of an Emblica extract, a Fucus extract, and a chebula extract, or a pharmaceutically acceptable form thereof, or one or more compounds constituent of one or more of an Emblica extract, a Fucus extract, and a chebula extract, or a pharmaceutically acceptable form thereof, or one or more compounds having a similarity score of at least 95% with a compound constituent of one or more of an Emblica extract, a Fucus extract, and a chebula extract, or a pharmaceutically acceptable form thereof.

In accordance with one aspect, there is provided a method of improving embryo development or improving embryo fertilization rate. The method may comprise transferring ooplasm from a donor oocyte to a recipient oocyte to be fertilized to form the embryo. The donor oocyte may have a mitochondrial DNA (mtDNA) content greater than the recipient oocyte. At least one of the donor subject, a recipient subject, and the embryo may have been administered a composition comprising an effective amount of one or more of an Emblica extract, a Fucus extract, a chebula extract, or a pharmaceutically acceptable form thereof, or one or more compounds constituent of one or more of an Emblica extract, a Fucus extract, and a chebula extract, or a pharmaceutically acceptable form thereof, or one or more compounds having a similarity score of at least 95% with a compound constituent of one or more of an Emblica extract, a Fucus extract, and a chebula extract, or a pharmaceutically acceptable form thereof.

In accordance with one aspect, there is provided a method of reducing, preventing, or delaying perimenopause, menopause, or a symptom thereof in a subject. The method may comprise administering to the subject a composition comprising an effective amount of one or more of an Emblica extract, a Fucus extract, and a chebula extract, or a pharmaceutically acceptable form thereof, or one or more compounds constituent of one or more of an Emblica extract, a Fucus extract, and a chebula extract, or a pharmaceutically acceptable form thereof, or one or more compounds having a similarity score of at least 95% with a compound constituent of one or more of an Emblica extract, a Fucus extract, and a chebula extract, or a pharmaceutically acceptable form thereof.

The method may comprise administering to the subject an effective amount of an Emblica extract, one or more compound constituent of an Emblica extract or having a similarity score of at least 95% with a compound constituent of an Emblica extract, or a pharmaceutically acceptable form thereof.

The method may comprise administering to the subject an effective amount of a Fucus extract, one or more compound constituent of an Fucus extract or having a similarity score of at least 95% with a compound constituent of an Fucus extract, or a pharmaceutically acceptable form thereof.

The method may comprise administering to the subject an effective amount of a chebula extract, one or more compound constituent of a chebula extract or having a similarity score of at least 95% with a compound constituent of a chebula extract, or a pharmaceutically acceptable form thereof.

In some embodiments, the Emblica extract is derived from Emblica officinalis.

In some embodiments, the compound constituent of the Emblica extract is a benzoic acid substituted with 1 to 5 hydroxy groups and optionally 1 to 3 O—(C1-C5 alkyl) or O—(C1-C5 alkenyl) groups, or a pharmaceutically acceptable form thereof, or a benzene substituted with —CH═CH—(CH2)a-C(O)OH, wherein a is 0 to 5, and 1 to 5 hydroxy groups, or a pharmaceutically acceptable form thereof, or a combination of the foregoing.

In some embodiments, the compound contained in the Emblica extract is gallic acid, vanillic acid, chlorogenic acid, 5 caffeic acid, syringic acid, coumaric acid, quercetin, Emblicanin A, Emblicanin B, punigluconin, pedunculagin, punicafolin, phyllanemblin, kaempferol, ellagic acid, chebulinic acid, chebulagic acid, punicalagin, a metabolite of any of the foregoing, a compound having a similarity score of at least 95% with any of the foregoing, or a pharmaceutically acceptable form of any of the foregoing.

In some embodiments, the Fucus extract is derived from Fucus vesiculosus, Fucus serratus, Fucus, spiralis, or Fucus guiryi.

In some embodiments, the compound constituent of the Fucus extract is a benzoic acid substituted with 1 to 5 hydroxy groups and optionally 1 to 3 O—(C1-C5 alkyl) or O—(C1-C5 alkenyl) groups, or a pharmaceutically acceptable form thereof, or a benzene substituted with —CH═CH—(CH2)a-C(O)OH, wherein a is 0 to 5, and 1 to 5 hydroxy groups, or a pharmaceutically acceptable form thereof, or a combination of the foregoing.

In some embodiments, the compound constituent of the Fucus extract is gallic acid, vanillic acid, chlorogenic acid, caffeic acid, syringic acid, coumaric acid, quercetin, fucoidan, punigluconin, pedunculagin, punicafolin, phyllanemblin, kaempferol, ellagic acid, chebulinic acid, chebulagic acid, punicalagin, a metabolite of any of the foregoing, a compound having a similarity score of at least 95% with any of the foregoing, or a pharmaceutically acceptable form of any of the foregoing.

In some embodiments, the chebula extract is derived from Terminilia chebula, Terminalia arborea, or Lumnitzera racemose.

In some embodiments, the compound constituent of the chebula extract is a benzoic acid substituted with 1 to 5 hydroxy groups and optionally 1 to 3 O—(C1-C5 alkyl) or O—(C1-C5 alkenyl) groups, or a pharmaceutically acceptable form thereof, or a benzene substituted with —CH═CH—(CH₂)a-C(O)OH, wherein a is 0 to 5, and 1 to 5 hydroxy groups, or a pharmaceutically acceptable form thereof, or a combination of the foregoing.

In some embodiments, the compound constituent of the chebula extract is gallic acid, vanillic acid, chlorogenic acid, caffeic acid, syringic acid, coumaric acid, quercetin, fucoidan, punigluconin, and pedunculagin, punicafolin, phyllanemblin, kaempferol, ellagic acid, chebulinic acid, chebulagic acid, punicalagin, a metabolite of any of the foregoing, a compound having a similarity score of at least 95% with any of the foregoing, or a pharmaceutically acceptable form of any of the foregoing.

In some embodiments, the composition comprises two or more of: an effective amount of an Emblica extract, or a pharmaceutically acceptable form thereof, or a compound constituent of an Emblica extract, or a pharmaceutically acceptable form thereof, or a compound having a similarity score of at least 95% with a compound constituent of an Emblica extract, or a pharmaceutically acceptable form thereof; an effective amount of a Fucus extract, or a pharmaceutically acceptable form thereof, or a compound constituent of a Fucus extract, or a pharmaceutically acceptable form thereof, or a compound having a similarity score of at least 95% with a compound constituent of a Fucus extract, or a pharmaceutically acceptable form thereof; and an effective amount of a chebula extract, or a pharmaceutically acceptable form thereof, or a compound constituent of a chebula extract, or a pharmaceutically acceptable form thereof, or a compound having a similarity score of at least 95% with a compound constituent of a chebula extract, or a pharmaceutically acceptable form thereof.

In some embodiments, the composition is fortified with one or more compounds constituent of an Emblica extract, or a pharmaceutically acceptable form thereof, or a compound having a similarity score of at least 95% with a compound constituent of an Emblica extract, or a pharmaceutically acceptable form thereof.

In some embodiments, the composition is fortified with one or more compounds constituent of a Fucus extract, or a pharmaceutically acceptable form thereof, or a compound having a similarity score of at least 95% with a compound constituent of a Fucus extract, or a pharmaceutically acceptable form thereof.

In some embodiments, the composition is fortified with one or more compounds constituent of a chebula extract, or a pharmaceutically acceptable form thereof, or a compound having a similarity score of at least 95% with a compound constituent of a chebula extract, or a pharmaceutically acceptable form thereof.

In some embodiments, the one or more compounds constituent of the Emblica extract or having a similarity score of at least 95% with a compound constituent of the Emblica extract is purified, e.g., at least 80% purified, at least 85% purified, at least 90% purified, at least 95% purified, at least 98% purified, at least 99% purified, at least 99.9% purified, at least 99.99% purified, or at least 99.999% purified.

In some embodiments, the one or more compounds constituent of the Fucus extract or having a similarity score of at least 95% with a compound constituent of the Fucus extract is purified, e.g., at least 80% purified, at least 85% purified, at least 90% purified, at least 95% purified, at least 98% purified, at least 99% purified, at least 99.9% purified, at least 99.99% purified, or at least 99.999% purified.

In some embodiments, the one or more compounds constituent of the chebula extract or having a similarity score of at least 95% with a compound constituent of the chebula extract is purified, e.g., at least 80% purified, at least 85% purified, at least 90% purified, at least 95% purified, at least 98% purified, at least 99% purified, at least 99.9% purified, at least 99.99% purified, or at least 99.999% purified.

In some embodiments, the effective amount is a therapeutically effective amount.

In some embodiments, administration induces mitochondrial biogenesis and/or improves mitochondrial function.

In some embodiments, the effective amount or therapeutically effective amount is sufficient to induce mitochondrial biogenesis.

In some embodiments, administration increases expression of at least one protein selected from PGC-1a, TFAM, NRF-1, and COX II.

In some embodiments, administration modulates menstrual cycles, e.g., normalizes menstrual cycles, increases ovulation events, and/or increases an anti-Mullerian hormone (AMH) level of the subject or recipient subject.

In some embodiments, administration decreases the probability of embryonic aneuploidy and/or Leigh's syndrome.

In some embodiments, administration reduces or lessens severity or frequency of at least one symptom of an ovarian diseases or conditions e.g., skin or vaginal dryness, pelvic pain or cramping, inflammation, prolonged or irregular menstrual cycles, and decreased ovulation events.

In some embodiments, the composition is administered topically.

In some embodiments, the composition is administered parenterally, e.g., intravenously, intraperitoneally, or intramuscularly.

In some embodiments, the composition is administered enterally.

In some embodiments, the composition is formulated as a topical solution, oil, cream, emulsion, or gel.

In some embodiments, the composition is formulated as a shampoo, conditioner, spray, cream, gel, balm, body wash, soap, lotion, or make-up.

In some embodiments, the composition is formulated as a parenteral liquid solution.

In some embodiments, the composition is formulated as an enteral capsule or tablet, or dietary supplement or food, e.g., food, food supplement, medical food, food additive, nutraceutical, or drink.

In some embodiments, the composition is administered locally.

In some embodiments, the composition is administered systemically.

In some embodiments, the composition is formulated for immediate release.

In some embodiments, the composition is formulated for extended release, e.g., controlled or sustained release.

The composition may be administered in combination with a standard of care treatment for improving fertility.

The composition may be administered in combination with a standard of care treatment for treating an ovarian disease or condition or a symptom thereof.

The composition may be administered in combination with one or more of clomiphene, tamoxifen, letrozole, metformin, gonadotrophins, gonadotrophin-releasing hormone, dopamine agonists, and other hormonal treatments.

The composition may be administered in combination with a surgical procedure, e.g., fallopian tube surgery, laparoscopic surgery to remove or destroy cysts or submucosal fibroids, or laparoscopic ovarian drilling.

The composition may be administered in combination with a UV-blocking agent, moisturizer, sunscreen, wrinkle cream, retinoid, alpha-hydroxy acid, beta-hydroxy acid, squalene, antioxidant, tretinoin, glycosaminoglycan (GAG), lactic acid, malic acid, citric acid, tartaric acid, hydroquinone, kojic acid, L-ascorbic acid, licorice extract, N-acetylglucosamine, niacinamide, soy, dermal filler or injection, e.g. hyaluronic acid or calcium hydroxylapatite, botulinum toxin, laser resurfacing procedure, ultrasound therapy, chemical peel, e.g. glycolic acid peel, trichloroacetic acid or salicylic acid, or dermabrasion procedure.

In some embodiments, the composition is administered in combination with a biomaterial, or encapsulated in a biomaterial, selected from an extracellular vesicle, collagen, hyaluronic acid, a synthetic biomaterial (e.g., polylactic acid (PLA), polyglycolic acid (PGA), polycaprolactone (PCL), polyethylene glycol (PEG)), fibrin, alginate, and a composite biomaterial.

In some embodiments, the ovarian disease or condition is selected from perimenopause, menopause, endometriosis, ovarian cysts, pre-menopausal or post-menopausal ovarian cancer, e.g., ovarian epithelial cancer, ovarian tumors, e.g., ovarian germ cell tumor, ovarian low malignant potential tumors, and ovarian stromal tumors, polycystic ovary syndrome (PCOS), primary ovarian insufficiency (POI), and ovarian torsion.

The composition may be administered in combination with one or more of hormone therapy, e.g., estrogen, progesterone, testosterone, or a synthetic form thereof, selective serotonin reuptake inhibitors (SSRIs), gabapentin, clonidine, hormonal contraceptives, gonadotropin-releasing hormone (GnRH) agonists or antagonists, heat therapy, pain relievers, nonsteroidal anti-inflammatory drugs (NSAID), such as ibuprofen or other analgesics, spironolactone, eflornithine, electrolysis, chemotherapy, radiation therapy, and surgery, e.g., hysterectomy, bilateral salpingo-oophorectomy, and debulking surgery, and lifestyle changes, such as weight loss, improved nutrition, and increased physical activity.

In some embodiments, the embryo may be produced by in vitro fertilization.

The method may comprise measuring mitochondrial content of the embryo and selecting the embryo responsive to the measurement of mitochondrial content being within a predetermined range.

The donor oocyte may be autologous.

The donor oocyte may be allogeneic.

The donor oocyte may be xenogeneic.

In some embodiments, the ooplasm transfer may be complete.

In some embodiments, the ooplasm transfer may be partial, e.g., by electrofusion or direct ooplasmic injection.

In some embodiments, the transfer may be performed by modified intracytoplasmic sperm injection (ICSI).

In some embodiments, the transfer may be performed by autonomous germline mitochondrial energy transfer (AUGMENT) on the oocyte.

The method may comprise using CRISPR/Cas 9 gene editing technology on the mtDNA of the donor oocyte or the recipient oocyte.

In some embodiments, the method may comprise using CRISPR/Cas 9 gene editing technology on mitochondrial DNA (mtDNA) of an oocyte of the subject to be fertilized to form the embryo.

The method may further comprise introducing stem cell generated mitochondrial DNA (mtDNA) into an oocyte of the subject to be fertilized to form the embryo.

The method may comprise introducing antioxidants to a culture media of the embryo during development.

In some embodiments, the transfer may be performed by nuclear genome transfer, e.g., oocyte spindle transfer, germinal vesicle (GV) transfer, pronuclear transfer (PNT), or polar body nuclear transfer (PBNT).

The subject or recipient subject may be characterized by age-related reduced fertility, infertility, and/or ovarian aging.

The subject or recipient subject may be 40 years or older.

The subject or recipient subject may be characterized by premature reproductive aging, e.g., primary ovarian insufficiency (POI), early onset prolonged or irregular menstrual cycles, early onset decreased ovulation events, premature menopause or perimenopause, and/or ovarian inflammation associated with premature reproductive aging.

The subject or recipient subject may be younger than 40 years.

The subject may be pre-menopausal.

The subject may be perimenopausal.

The subject may be post-menopausal.

The subject or recipient subject may have a diminished ovarian reserve (DOR).

The subject or recipient subject may have a normal ovarian reserve (NOR).

The subject or recipient subject may suffer from one or more of obesity, diabetes, chronic inflammation, high blood sugar, an autoimmune disease, and/or poor lifestyle factors, such as smoking, alcohol use, drug use, exposure to chemicals (e.g., bisphenol A, advanced glycation end products (AGE)) and/or radiation, poor nutrition, e.g., increased ingestion of charred foods, increased ingestion of saturated fats, and/or reduced ingestion of fruits and vegetables, and/or reduced exercise.

The subject or recipient subject may be characterized by a reduced anti-Mullerian hormone (AMH) level, e.g., less than about 80 ng/mL, an increased follicle stimulating hormone (FHL) level, e.g., greater than about 10 IU/L, an increased estradiol level, and/or a reduced antral follicle count (AFC), e.g., less than about 5-7 total follicles.

The subject or recipient subject may be undergoing or have attempted in vitro fertilization (IVF).

The subject or recipient subject may be a poor responder to IVF.

The subject or recipient subject may be a normal responder to IVF.

The subject or recipient subject may suffer from reduced production of reproductive hormones, e.g., estrogen and/or progesterone, increased production of follicle-stimulating hormone (FSH), prolonged or irregular menstrual cycles, uterine or vaginal atrophy, e.g., decreased endometrial glands, diffuse glandular breakdown (apoptotic bodies) of the uterus, acute inflammation of the uterus, and/or decreased vaginal epithelial thickness, decreased ovulation events, ovarian inflammation, and/or infertility.

The subject or recipient subject may be characterized by a mitofusin (Mfn1) gene abnormality, a dynamic related protein 1 (Drp1) gene abnormality, a caseinolytic peptidase P (Clpp) gene abnormality, a growth differentiation factor 9 (GDF9) gene abnormality, a fragile-X mental retardation 1 (FMR1) gene abnormality, a transcription factor A (TFAM) gene abnormality, a mitochondrial DNA polymerase gamma (PolgA) gene abnormality, a mitochondrial inner membrane peptidase (IMP) gene abnormality, and/or a mitochondrial ABC transporter (MDR-1) gene abnormality.

The subject or recipient subject may be characterized by decreased gene expression of PPARy, PGC-1a, PGC-1b, ERR, NRF-1, NRF-2, SIRT1, SIRT3, SIRT4, and/or SIRT5.

The subject or recipient subject may be characterized by decreased AMP-activated protein kinase (AMPK) and or PTEN-induced kinase 1 (PINK1) protein expression and/or increased mammalian target of rapamycin (mTOR) protein expression.

The subject or recipient subject may have a somatic mitochondrial DNA mutation in an oocyte to be fertilized to form the embryo or the recipient oocyte, e.g., a T414G transverse mutation.

The subject or recipient subject may have an inherited mitochondrial DNA (mtDNA) mutation in an oocyte to be fertilized to form the embryo or the recipient oocyte, e.g., a T414G transverse mutation.

The subject or recipient subject may be characterized by decreased levels of mitochondrial DNA (mtDNA) in an oocyte to be fertilized to form the embryo or the recipient oocyte and/or reduced oocyte quality, e.g., chromosomal misalignment and/or spindle malformation.

The subject or recipient subject may be characterized by decreased gene expression of superoxide dismutase (SOD) and/or increased levels of Aldh3A2 enzyme.

The subject or recipient subject may be characterized by decreased mRNA expression of Prdx3, Prdx4, and/or Txn2 enzyme.

The subject or recipient subject may suffer from Turner's Syndrome, galactosemia, and/or Fragile X Syndrome.

The subject or recipient subject may be characterized by progressive external ophthalmoplegia (POE), spinocerebellar ataxia with epilepsy (SCAE), and/or mitochondrial encephalomyopathy, lactic acidosis, and stroke-like episodes (MELAS).

The subject or recipient subject may be characterized by a neurologic condition.

The subject or recipient subject may be characterized by dysfunctional folliculogenesis, e.g., follicle depletion or follicle dysfunction.

The subject or recipient subject may have decreased tertiary follicle counts, e.g., early antral, antral, and/or pre-ovulatory, and/or decreased corpora lutea (CL) follicle counts.

The subject or recipient subject may be characterized by down-regulation of estrogen receptors (ER) α and β in the ovary, reduced ESR1 or ESR2 gene expression, increased miR-206-3p RNA expression, reduced ovarian production of 17β-estradiol (E2), and/or increased lipid-laden ovarian stromal cells.

In some embodiments, the composition comprises a nanoparticle-based delivery carrier.

In some embodiments, the composition comprises a skin penetration enhancer or is administered in combination with a skin penetration enhancer, e.g., a chemical skin penetration enhancer or a physical skin penetration enhancer.

In some embodiments, the composition comprises a mitochondria-targeting agent or a delivery carrier functionalized with a mitochondria-targeting agent.

In some embodiments, the compound constituent of an Emblica extract was derived from, purified from, or isolated from the Emblica extract.

In some embodiments, the compound constituent of an Emblica extract was derived from, purified from, or isolated from a source other than the Emblica extract.

In some embodiments, the compound constituent of an Emblica extract was synthesized.

In some embodiments, the compound constituent of a Fucus extract was derived from, purified from, or isolated from the Fucus extract.

In some embodiments, the compound constituent of a Fucus extract was derived from, purified from, or isolated from a source other than the Fucus extract.

In some embodiments, the compound constituent of a Fucus extract was synthesized.

In some embodiments, the compound constituent of a chebula extract was derived from, purified from, or isolated from the chebula extract.

In some embodiments, the compound constituent of a chebula extract was derived from, purified from, or isolated from a source other than the chebula extract.

In some embodiments, the compound constituent of a chebula extract was synthesized.

In accordance with another aspect, there is provided a kit comprising a preparation comprising an effective amount of donor mtDNA in a therapeutically acceptable carrier. The kit may comprise a composition comprising an effective amount of one or more of an Emblica extract, a Fucus extract, and a chebula extract, or a pharmaceutically acceptable form thereof, or one or more compounds constituent of one or more of an Emblica extract, a Fucus extract, and a chebula extract, or a pharmaceutically acceptable form thereof, or one or more compounds having a similarity score of at least 95% with a compound constituent of one or more of an Emblica extract, a Fucus extract, and a chebula extract, or a pharmaceutically acceptable form thereof.

The disclosure contemplates all combinations of any one or more of the foregoing aspects and/or embodiments, as well as combinations with any one or more of the embodiments set forth in the detailed description and any examples.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A shows quantification of immunofluorescence analysis of OXPHOS complex IV (COXII) in relative fluorescence units (RFUs) in paraffin dorsal skin sections control and mtDNA-depleter mice after dox induction and 16 weeks of topical Emblica extract treatment or control treatment. Data are mean±SD (n=3); * P<0.05;

FIG. 1B shows RT-PCR analysis of dorsal skin RNA for mtDNA encoded genes from control and mtDNA-depleter mice after dox induction and 16 weeks of topical Emblica extract treatment or control treatment;

FIG. 1C shows quantification of mtDNA content (mean±s.e.m; * P<0.05, Student's t test) in dorsal skin samples from control (n=3) and mtDNA-depleter (n=3) mice after dox induction and 16 weeks of topical Emblica extract treatment or control treatment application;

FIG. 1D shows quantitative analysis of inflammatory cells in the skin sections from control and mtDNA-depleter mice after dox induction and 16 weeks of topical Emblica extract treatment or control treatment (mean±SD; * P<0.05; ** P<0.01; *** P<0.001 Student's t test);

FIG. 2A shows representative pictures of: (i) control mice after dox induction; (ii) mtDNA-depleter mice after dox-induction; (iii) control mice after dox induction and 51 days of treatment with Fucus extract; and (iv) mtDNA-depleter mice after dox induction and 51 days of treatment with Fucus extract (preventive intervention) (n=3 for each group);

FIG. 2B shows quantification of mtDNA content (mean±s.e.m; * P<0.05, Student's t test) in dorsal skin samples from control (n=3) and mtDNA-depleter (n=3) mice after dox induction and 16 weeks of topical Fucus extract treatment or control treatment application;

FIG. 3 shows schematics of Emblica extract preventive and therapeutic in vivo experiments;

FIG. 4A is a graph showing induced expression of mitochondrial complex IV subunit 2 (COXII) by various compositions, according to one embodiment;

FIG. 4B is a graph showing induced expression of mitochondrial transcription factor A (TFAM) by various compositions, according to one embodiment;

FIG. 4C is a graph showing induced expression of peroxisome proliferator-activated receptor gamma coactivator 1-alpha (PGC-1a) by various compositions, according to one embodiment;

FIG. 5 is an image of an electrophoresis gel showing protein levels of the mitochondrial biogenesis markers COXII, TFAM, and PGC-1a induced by various compositions, according to one embodiment;

FIGS. 6A-6F are graphs showing COXII expression induced by administration of an Emblica extract in vitro at 6 hours to 96 hours after administration;

FIGS. 7A-7F are graphs showing TFAM expression induced by administration of an Emblica extract in vitro at 6 hours to 96 hours after administration;

FIGS. 8A-8F are graphs showing PCG-1a expression induced by administration of an Emblica extract in vitro at 6 hours to 96 hours after administration;

FIGS. 9A-9D are graphs showing COXII expression induced by administration of an Emblica extract in vitro, optionally with a nanoparticle delivery carrier, at 6 hours and 24 hours after administration;

FIGS. 10A-10B include graphs showing expression of COXII and TFAM at 6 hours after in vitro administration of a constituent of Emblica extract, according to one embodiment;

FIGS. 11A-11D include graphs showing expression of COXII and TFAM at 12 hours and 24 hours after in vitro administration of a constituent of Emblica extract, according to one embodiment;

FIGS. 12A-12D include graphs showing expression of COXII and TFAM at 12 hours and 24 hours after in vitro administration of a constituent of Emblica extract, according to one embodiment;

FIGS. 13A-13D include graphs showing expression of COXII and TFAM at 12 hours and 24 hours after in vitro administration of a constituent of Emblica extract, according to one embodiment;

FIGS. 14A-14D include graphs showing expression of COXII and TFAM at 12 hours and 24 hours after in vitro administration of a constituent of Emblica extract, according to one embodiment;

FIGS. 15A-15D include graphs showing expression of COXII at 6, 12, 24, and 48 hours after in vitro administration of a constituent of Emblica extract, according to one embodiment;

FIGS. 16A-16D include graphs showing expression of TFAM at 6, 12, 24, and 48 hours after in vitro administration of a constituent of Emblica extract, according to one embodiment;

FIGS. 17A-17D include graphs showing expression of COXII at 6, 12, 24, and 48 hours after in vitro administration of a constituent of Emblica extract, according to one embodiment;

FIGS. 18A-18D include graphs showing expression of TFAM at 6, 12, 24, and 48 hours after in vitro administration of a constituent of Emblica extract, according to one embodiment;

FIGS. 19A-19D include graphs showing expression of COXII at 6, 12, 24, and 48 hours after in vitro administration of a constituent of Emblica extract, according to one embodiment;

FIGS. 20A-20D include graphs showing expression of TFAM at 6, 12, 24, and 48 hours after in vitro administration of a constituent of Emblica extract, according to one embodiment;

FIGS. 21A-21D include graphs showing expression of COXII at 6, 12, 24, and 48 hours after in vitro administration of a constituent of Emblica extract, according to one embodiment;

FIGS. 22A-22D include graphs showing expression of TFAM at 6, 12, 24, and 48 hours after in vitro administration of a constituent of Emblica extract, according to one embodiment;

FIGS. 23A-23D include graphs showing expression of COXII at 6, 12, 24, and 48 hours after in vitro administration of a chebulinic acid, optionally encapsulated in a nanoparticle carrier, according to one embodiment;

FIGS. 24A-24D include graphs showing expression of TFAM at 6, 12, 24, and 48 hours after in vitro administration of chebulinic acid, optionally encapsulated in a nanoparticle carrier, according to one embodiment;

FIGS. 25A-25B include graphs showing expression of COXII at 6 hours after in vitro administration of Emblica extract encapsulated in a nanoparticle carrier, according to one embodiment;

FIGS. 26A-26D include graphs showing expression of COXII at 6, 12, 24, and 48 hours after in vitro administration of Emblica extract encapsulated in a nanoparticle carrier, according to one embodiment;

FIGS. 27A-27D include graphs showing expression of TFAM at 6, 12, 24, and 48 hours after in vitro administration of Emblica extract encapsulated in a nanoparticle carrier, according to one embodiment;

FIGS. 28A-28D include graphs showing expression of COXII at 6, 12, 24, and 48 hours after in vitro administration of Emblica extract encapsulated in a nanoparticle carrier, according to one embodiment;

FIGS. 29A-29D include graphs showing expression of COXII at 6, 12, 24, and 48 hours after in vitro administration of Emblica extract encapsulated in a nanoparticle carrier, according to one embodiment; and

FIGS. 30A-30D include graphs showing expression of TFAM at 6, 12, 24, and 48 hours after in vitro administration of Emblica extract encapsulated in a nanoparticle carrier, according to one embodiment.

### DETAILED DESCRIPTION

Mitochondrial dysfunction is associated with many mitochondrial diseases, many of which are the result of dysfunctional mitochondrial oxidative phosphorylation (OXPHOS). Mitochondrial OXPHOS accounts for the generation of most of the cellular adenosine triphosphate (ATP) in a cell. The OXPHOS function largely depends on the coordinated expression of proteins encoded by both nuclear and mitochondrial genomes. The human mitochondrial genome encodes for 13 polypeptides of the OXPHOS system, and the nuclear genome encodes the remaining more than 85 polypeptides required for the assembly of OXPHOS system. mtDNA depletion impairs OXPHOS and leads to mtDNA depletion syndromes (Alberio, et al., Mitochondrion 7, 6-12, 2007; Ryan, M et al., Annu. Rev. Biochem. 76, 701-722, 2007). The mtDNA depletion syndromes are a heterogeneous group of disorders, characterized by low mtDNA levels in specific tissues. In different target organs, mtDNA depletion leads to specific pathological changes (Tuppen, et al., Biochim. Biophys. Acta 1797, 113-128, 201)). mtDNA depletion syndromes result from the genetic defects in the nuclear-encoded genes that participate in mtDNA replication, and mitochondrial nucleotide metabolism and nucleotide salvage pathway (Alberio, et al., Mitochondrion 7, 6-12, 2007). mtDNA depletion is also implicated in other human diseases and conditions, such as, but not limited to, mtDNA depletion syndromes, mitochondrial diseases, viral infections, viral infection-induced symptoms, aging, aging-associated chronic diseases or conditions, reduced energy levels and vitality, characteristics of hair aging including hair loss and graying, characteristics of skin aging including skin wrinkles and senile lentigines, skin diseases and conditions, and other human pathologies.

Decreases in fertility and oocyte mitochondrial mass typically occur with aging. Embryo development and embryo fertilization rate may also decline with advancing age. Additionally, advanced age individuals are often at greater risk of ovarian diseases or disorders. It is also hypothesized that a decrease in fertility or oocyte mitochondrial mass, declining embryo development and embryo fertility rate, as well as certain ovarian diseases and disorders may occur as a result of premature aging. For instance, primary ovarian insufficiency (POI) is a disorder associated with reduced ovarian function before the age of 40.

A general decline in mitochondrial function has been extensively reported during aging and mitochondrial dysfunction is known to be a driving force underlying age-related human diseases. A mouse that carries a specific mtDNA mutation has been shown to present signs of premature aging (i.e., a mtDNA depleter mouse). In addition to mutations in mtDNA, studies also suggest a decrease in mtDNA content and mitochondrial copy number with age. Low mtDNA copy number is linked to frailty and, for a multiethnic population, is a predictor of all-cause mortality. A recent study revealed that humans on an average lose about four copies of mtDNA every ten years. This study also identified an association of decrease in mtDNA copy number with age-related physiological parameters.

Accumulating evidence suggests a strong link between mitochondrial dysfunction, mitochondrial diseases, aging, and aging-associated diseases. Notably, increased somatic mtDNA mutations and decline in mitochondrial functions have been extensively reported during human aging. Studies also suggest a decrease in mtDNA content and mitochondrial number with age.

### Definitions

As used herein, the term “carrier” refers to a diluent or vehicle with which a compound is administered. The carrier may be a pharmaceutically acceptable carrier. The carrier may be a cosmetically acceptable carrier. Examples of suitable pharmaceutical carriers are described in “Remington's Pharmaceutical Sciences” by E. W. Martin.

As used herein, the term “corresponding aspartic acid” means an aspartic acid (D) residue that is mutated to an alanine (A) residue in a POLG1 amino acid sequence that is the equivalent of the aspartic acid at position 1135 of the human POLG1 sequence. In a particular embodiment, the “corresponding aspartic acid” is flanked on the amino terminus side by an amino acid sequence of S/T I/V H X, I S/T I/V H X, or C/A I S/T I/V H X, and/or on the carboxy terminus side by an amino acid sequence of X E V/IR, X E V/IR Y/F, or X E V/I R Y/F L, wherein “X” indicates the aspartic acid amino acid that is mutated or will be mutated to alanine.

As used herein, the terms “depleted.” “depletion,” or “depleter” with respect to mtDNA refers to a decrease in mtDNA copy number and/or concentration in a human or in a non-human animal, tissue, or cell of the non-human animal. Such a determination may be made with regard to a human that has not been administered a compound or composition of the disclosure or to a control non-human animal tissue, or cell (for example, a non-human animal that has not expressed or does not express a mutant POLG1 polypeptide).

As used herein, “treatment” of a disease or condition refers to reducing or lessening the severity or frequency of at least one symptom of that disease or condition, compared to a similar but untreated patient. Treatment can also refer to halting, slowing, or reversing the progression of a disease or condition, compared to a similar but untreated patient. Treatment may comprise addressing the root cause of the disease and/or one or more symptoms.

As used herein, the term “effective amount” refers to an amount of a compound or composition of the disclosure administered to a subject, which is effective to provide a desired response and/or effect in the subject. The response and/or effect may be a cosmetic response and/or effect. The response and/or effect may be a therapeutic response and/or effect. The response and/or effect may be a prophylactic or preventative response and/or effect.

As used herein, the term “therapeutically effective amount” refers to an amount of compound or composition of the disclosure administered to a subject, which is effective to treat a disease or condition described herein in a subject and/or to produce a desired physiological response and/or therapeutic effect in the subject. One example of a desired physiological response includes increasing mtDNA copy number and/or concentration.

The actual dose which comprises the “effective amount” or “therapeutically effective amount” may depend upon the route of administration, the size and health of the subject, the disorder being treated, and the like.

In some embodiments, the “effective amount” or “therapeutically effective amount” in the context of the present disclosure may be sufficient to treat or prevent ovarian diseases or conditions and symptoms thereof.

In some embodiments, the “effective amount” or “therapeutically effective amount” in the context of the present disclosure is sufficient to induce mitochondrial biogenesis. The “effective amount” or “therapeutically effective amount” may be sufficient to induce mitochondrial biogenesis locally. The “effective amount” or “therapeutically effective amount” may be sufficient to induce mitochondrial biogenesis systemically.

In some embodiments, the “effective amount” or “therapeutically effective amount” in the context of the disclosure decreases an inflammatory phenotype, increases expression of mitochondrial oxidative phosphorylation complexes, increases stability of mitochondrial oxidative phosphorylation complexes, alters, e.g., decreases expression of at least one gene selected from the group consisting of: NF-κB, COX-2, INF-β1, CCL5, MMP1, MMP2, MMP9, MMP13, IGF1R, VEGF, and MRPS5, alters, e.g., increases expression of at least one gene selected from the group consisting of: TIMP1 KLOTHO, COL1A1, MTCO2, TFAM, and VDAC, prevents a deactivation of a gene associated with mitochondrial health and activity, selected from: FGF2, FGFR1, COX7A1, PDK4, FAM173A, MRPL12, and WNT11, decreases inflammatory infiltrate in the skin and/or hair follicles, and/or increases expression of at least one protein selected from PGC-1a, TFAM, NRF-1, and COXII.

In some embodiments, the “effective amount” or “therapeutically effective amount” in the context of the disclosure increases mtDNA copy number or concentration by at least 5%, for example, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%. In each of the foregoing, when a reduction or increase is specified, such reduction or increase may be determined with respect to a subject or non-human animal that has not been treated with a compound or composition of the disclosure and that is suffering from a disease or condition described herein.

In some embodiments, the “effective amount” or “therapeutically effective amount” in the context of the disclosure prevents incidence of an inflammatory phenotype, prevents a decrease in expression of mitochondrial oxidative phosphorylation complexes, prevents a decrease in stability of mitochondrial oxidative phosphorylation complexes, prevents an increase in expression of at least one gene selected from the group consisting of: NF-κB, COX-2. INF-β1, CCL5, MMP1, MMP2, MMP9, MMP13, IGF1R, VEGF, and MRPS5, prevents a decrease in expression of at least one gene selected from the group consisting of: TIMP1 KLOTHO, COL1A1, MTCO2, TFAM, and VDAC, prevents a deactivation of a gene associated with mitochondrial health and activity, selected from: FGF2, FGFR1, COX7A1, PDK4, FAM173A, MRPL12, and WNT11, prevents an increase in inflammatory infiltrate in the skin and/or hair follicles, prevents a decrease in mtDNA copy number or concentration, and/or prevents a decrease in expression of at least one protein selected from PGC-1a, TFAM, NRF-1, and COXII.

As used herein, the term “excipient” means a substance formulated alongside the active ingredient of a composition included for purposes such as, but not limited to, long-term stabilization, bulking up solid formulations that contain potent active ingredients in small amounts (thus often referred to as bulking agents, fillers, or diluents), or to confer a therapeutic enhancement on the active ingredient in the final dosage form, such as facilitating drug absorption, reducing viscosity, or enhancing solubility. Excipients can also be useful in the manufacturing process, to aid in the handling of the active substance concerns such as by facilitating powder flowability or non-stick properties, in addition to aiding in vitro stability such as prevention of denaturation or aggregation over the expected shelf life. The excipient may be a pharmaceutically acceptable excipient. The excipient may be a cosmetically acceptable excipient. Examples of suitable excipients are described in “Remington's Pharmaceutical Sciences” by E. W. Martin.

As used herein, the term “in need of” (such as in the phrase “in need of treatment”) refers to a judgment made by a healthcare professional that a subject requires or will benefit from administration of a compound of the disclosure. This judgment is made based on a variety of factors that are in the realm of a healthcare professional's expertise, such as, but not limited to, the knowledge that the subject is ill, or will be ill, as the result of a disease or condition that is treatable by a method or drug composition of the disclosure.

As used herein, the terms “mutant POLG1” or “mutated POLG1” refers to a POLG1 amino acid sequence from a particular species that contains at least one mutation as compared to the wild-type POLG1 sequence from that species. A mutation need not cause a disease. A single mutation or more than one mutation may be present. In a particular embodiment, a single dominant negative mutation may be present (such as, but not limited to, a D1135A mutation), optionally with one or additional mutations.

As used herein, the term “pharmaceutically acceptable” refers to a compound that is compatible with the other ingredients of a composition and not deleterious to the subject receiving the compound or composition. In some embodiments, the term “pharmaceutically acceptable” means approved by a regulatory agency of the Federal or a state government or listed in the U.S. Pharmacopeia or other generally recognized pharmacopeia for use in animals, and more particularly in humans.

As used herein, the term “cosmetically acceptable” refers to a compound that is compatible with the other cosmetic ingredients of a composition and not deleterious to the subject receiving the compound or composition. A cosmetically acceptable composition or compound may be pharmaceutically acceptable. However, a cosmetically acceptable composition or compound need not be pharmaceutically acceptable.

As used herein, the term “pharmaceutically acceptable form” is meant to include known forms of a compound of the disclosure that may be administered to a subject, including, but not limited to, solvates, hydrates, prodrugs, isomorphs, polymorphs, pseudomorphs, neutral forms and salt forms of a compound of the disclosure.

As used herein, the term “pharmaceutically acceptable salt” refers to physiologically and pharmaceutically acceptable salts of the compounds of the invention: i.e., salts that retain the desired biological activity of the parent compound and do not impart undesired toxicological effects to the compounds disclosed. For oligonucleotides, exemplary pharmaceutically acceptable salts include but are not limited to (a) salts formed with cations such as sodium, potassium, ammonium, magnesium, calcium, polyamines such as spermine and spermidine and the like; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

As used herein, the term “pharmaceutical composition” refers to a mixture of one or more of the compounds of the disclosure, with other components, such as, but not limited to, pharmaceutically acceptable carriers and/or excipients. The purpose of a pharmaceutical composition is to facilitate administration of a compound of disclosure.

In some embodiments, a “cosmetically acceptable” excipient refers to a cosmetically acceptable material, composition, or vehicle, such as a liquid or solid filler, diluent, solvent, or encapsulating material. In some embodiments, each excipient is cosmetically acceptable in the sense of being compatible with the other ingredients of a cosmetic formulation, and suitable for use in contact with the tissue or organ of humans and animals without excessive toxicity, irritation, allergic response, immunogenicity, or other problems or complications, commensurate with a reasonable benefit/risk ratio.

As used herein, the terms “repleted,” “repletion,” or “repleter” with respect to mtDNA refers to an increase in mtDNA copy number and/or concentration in a human or a non-human animal, tissue, or cell. Such a determination may be made with regard to a human or to a control non-human animal tissue, or cell that has undergone mtDNA depletion (such as immediately before repletion is initiated. In certain aspects, mtDNA is repletion results in mtDNA copy number and/or concentration approximately equal to the mtDNA copy number and/or concentration observed in a control non-human animal, tissue, or cell (for example, a non-human animal that has not expressed or does not express a mutant POLG1 polypeptide).

As used herein, the term “solvate” means a compound of the disclosure, or a pharmaceutically acceptable salt thereof, wherein one or more molecules of a suitable solvent are incorporated in the crystal lattice. A suitable solvent is physiologically tolerable at the dosage administered. Examples of suitable solvents are ethanol, water and the like. When water is the solvent, the molecule may be referred to as a “hydrate.”

As used herein, the terms “subject” or “patient” include all members of the animal kingdom including, but not limited to, vertebrates, mammals, animals (e.g., cats, dogs, horses, swine, rodents, etc.) and humans. In certain embodiments, the subject is a human. In certain embodiments, the subject is an animal, e.g., a research animal, e.g., a mouse, a rat.

As used herein, the terms “similarity” or “similarity score” when used with respect to a compound refers to a degree of similarity between two or more compounds in chemical structure, chemical function, and/or one or more chemical properties. The similarity score may be quantified by augmenting data generated by a deep neural network trained to model a plurality of chemical reactions for any given compound. The data may be augmented by a multi-dimensional vector defining a matrix of properties of the compound or a chemical reaction involving the compound to generate an embedding score for the compound. The embedding score for two or more compounds may be compared to generate the similarity score between the two or more compounds.

All patent applications, patents, and printed publications cited herein are incorporated herein by reference in their entireties, for all purposes.

### Impact of Mitochondrial Function on Intrinsic and Extrinsic Aging

Mitochondrial dysfunction is implicated in both intrinsic and extrinsic aging. The presence of skin wrinkles, acanthosis, epidermal hyperplasia with hyperkeratosis, and marked inflammatory infiltrate in the skin has been observed in mtDNA-depleter mice and represent characteristics similar to the extrinsic aging of skin in humans. Furthermore, the changes in expression of intrinsic aging-associated genetic markers support intrinsic mechanisms underlying the phenotypic changes observed in mtDNA-depleter mice.

Loss of collagen fibers is reported to underlie skin wrinkles. A tight balance between the proteolytic matrix metalloprotease (MMP) enzymes and their tissue-specific inhibitor tissue inhibitor metalloproteinase-1 (TIMP1) is essential to maintain the collagen fiber content in the skin. Expression of MMPs is altered in the aged skin. Consistent with these reports, the skin of mtDNA-depleter mice showed increased expression of MMPs and decreased expression of TIMP1, indicating loss of balance contributing to the development of skin wrinkles. Repletion of mtDNA content restored MMP expression leading to a reversal of wrinkled skin and hair loss. These experiments show that mitochondria are regulators of aging. This observation is surprising and suggests that epigenetic mechanisms underlying mitochondria-to-nucleus cross-talk must play an important role in the restoration of normal skin and hair phenotype.

mtDNA stress triggers inflammatory response. Inflammation also underlies aging and age-related diseases. Increased levels of markers of inflammation in the mtDNA-depleter mice indicate an activated immune response in the skin of mtDNA-depleter mice. Increased expression of NF-κB, a master regulator of the inflammatory response upon mtDNA depletion and its reduced expression after the restoration of mtDNA content suggests that NF-κB signaling is a critical mechanism contributing to the skin and hair follicle pathologies observed in mtDNA-depleter mice. Furthermore, a unique feature of proteins encoded by mtDNA is N-formyl-methionine at the N terminus. N-formylated peptides when present in the extracellular space are known to act as mitochondrial damage-associated molecular patterns and activate neutrophils or activate keratinocyte-intrinsic responses resulting in the recruitment of immune cells. While previous animal models have shown alternations in mtDNA homeostasis and/or mtDNA copy number or concentration using a localized approach (for example targeting only a specific cell type), the disclosure utilizes an animal model that provides for the global and targeted disruption of mtDNA homeostasis and/or mtDNA copy number of concentration in a controlled manner. Using such an animal model, the disclosure identified compounds that are effective in treating diseases and conditions relating to mitochondrial dysfunction in a background of significant mtDNA depletion. In addition, the disclosure demonstrates that such compounds reverse the physiological and phenotypic effects mediated by mitochondrial dysfunction. Exemplary physiological and phenotypic effects of intrinsic and extrinsic aging reversed include a decrease in skin wrinkles, a decrease in hair loss, an increase in hair follicles in growth phase, decreased inflammatory gene expression, decreased inflammatory infiltrate in the skin and hair follicles, and increased collagen content in the skin.

Further, due to the short lifespan of the animal models of the conventional practice, prior studies were prevented from determining the effect of mitochondrial dysfunction on the aging process and such studies did not observe the appearance of many of the physiological and phenotypic changes related to mitochondrial dysfunction. As such, the conventional practice was not capable of identifying compounds effective to treat such physiological and phenotypic changes related to mitochondrial dysfunction reported herein.

The foregoing limitations make identifying effective treatments for the skin diseases and conditions where mitochondrial dysfunction is at issue difficult. In addition, due to aberrations in normal epithelial development and hair follicle morphogenesis in current models, the issue of false positive and false negative results is high. The disclosure utilized an inducible non-human animal model expressing a mutated POLG1 polypeptide (such as, but not limited to, a POLG1 polypeptide expressing a dominant-negative (DN) mutation) that induces mitochondrial dysfunction (for example by depletion of mtDNA) in the whole animal or selected cells/tissues. The non-human animal model allows for the ubiquitous suppression and restoration of mitochondrial function in the whole animal or in specific cells/tissues.

The non-human animal model disclosed can be used to rapidly identify compounds effective in treating mtDNA-related diseases and conditions. The animal model in the absence of the expression of mutated POLG1 expression maintained normal epidermal differentiation and hair follicle morphogenesis making this animal model system a valuable tool in identifying therapies for the treatment of a variety of diseases and conditions characterized by mitochondrial dysfunction. When the mutant POLG1 polypeptide is expressed, the animal model demonstrates profound phenotypic age-related changes in the skin, including development of skin wrinkles and hair loss. The phenotypic changes are reversible when mitochondrial function is restored (such as through the administration of an extract or compound described herein).

### Non-Human Animal Model

In certain aspects, the methods described herein utilize a genetically modified non-human animal that expresses a mutant POLG1 polypeptide in a controlled manner. Tissues, organs and cells from such animal model may also be used. In one embodiment, the mutant POLG1 polypeptide is expressed ubiquitously (in every cell of the non-human animal). In another embodiment, the mutant POLG1 polypeptide is expressed in a specific tissue or set of tissues or in a specific cell type. Such non-human animal model is described in US Patent Publication No. 2020-0085021-A1, incorporated herein by reference in its entirety for all purposes.

In a particular aspect, the animal model exhibits at least one characteristic selected from the group consisting of: reduced mitochondrial (mt) DNA content, reduced mtDNA copy number, changes in mitochondrial protein expression, reduced expression of mitochondrial oxidative phosphorylation complexes, reduced stability of mitochondrial oxidative phosphorylation complexes, skin wrinkles, hair loss, increased epidermal thickness, epidermal hyperplasia, acanthosis, hyperkeratosis, altered, e.g., increased expression of at least one gene selected from the group consisting of: NF-κB, COX-2, INF-1, CCL5, MMP1, MMP2, MMP9, MMP13, IGF1R, VEGF, and MRPS5, altered, e.g., decreased expression of at least one gene selected from the group consisting of: TIMP1 and KLOTHO, COL1A1, MTCO2, TFAM, and VDAC, increased skin inflammation, and aberrant hair follicles.

### Methods of Screening for Therapeutic Agents

The disclosure provides an artificial neural network trained to model a plurality of chemical reactions for any given compound. Through the use of the artificial neural network described herein, compounds and compositions having a similarity score with a known promoter or inhibitor of mitochondrial biogenesis may be reliably identified. The similarity score may be quantified by augmenting data generated by the deep neural network in a multi-dimensional vector defining a matrix of properties of the compound or a chemical reaction involving the compound to generate an embedding score for the compound. The embedding score for the compound may be compared to an embedding score for the known promotor or inhibitor of mitochondrial biogenesis to generate the similarity score. Therefore, the disclosure provides for methods of screening compounds and compositions having a sufficient similarity score with known compounds. The identified compounds are predicted to be effective at promoting or inhibiting mitochondrial biogenesis.

The disclosure provides a non-human animal model comprising a mutant POLG1 polypeptide and that express the mutant POLG1 polypeptide in a controlled manner either throughout the animal or in specific tissues. Through the use of the non-human animal model described herein, compounds and compositions effective in treating diseases and conditions related to mitochondrial dysfunction can be reliably identified. Methods utilizing cells, and tissues from such a non-human animal are also provided. Therefore, the disclosure provides for methods of screening compounds and compositions effective to treat diseases and conditions related to mitochondrial dysfunction in a subject, including diseases and conditions related to mtDNA depletion.

In one embodiment, the disclosure provides for identification of a compound for the treatment of a disease or condition due, at least in part, to mitochodrial dysfunction, including changes in mtDNA copy number and/or concentration, and/or dysfunctional mitochondrial OXPHOS. Such diseases and conditions include, but are not limited to, mtDNA depletion syndromes, mitochondrial diseases, ovarian diseases or conditions and symptoms thereof, aging, aging-associated chronic diseases, reduced energy levels and vitality, and other human pathologies. Exemplary mitochondrial diseases include cardiovascular disease, diabetes, cancer, neurological disorders, such as age-associated neurological disorders, skin diseases and conditions, e.g., skin wrinkles, changes in skin pigmentation, senile lentigines, characteristics of skin aging, hair or scalp diseases and conditions, e.g., hair loss, hair thinning, changes in hair pigmentation, e.g., hair graying.

In one embodiment, such a method of screening comprises the steps of: a) providing a deep neural network trained to model a plurality of chemical reactions for a known promoter or inhibitor of mitochondrial biogenesis; b) executing the deep neural network to identify one or more compounds having a threshold similarity score of at least 70% with the known promoter or inhibitor of mitochondrial biogenesis; c) selecting one or more identified compounds on the basis of at least one inclusion criteria; and d) evaluating the at least one selected compound in an assay to determine an effect on mitochondrial biogenesis by the selected compound, wherein the assay may include an in vitro assay, ex vivo assay, or in vivo assay. The at least one inclusion criteria may include a structural, functional, physical, or chemical property of the identified compound. Exemplary properties include size, charge, ionic strength, bond strength, valence, hybridization, micromolecular structure, macromolecular structure, and others. The threshold similarity score may be at least 70%, at least 75%, at least 80%, at least 85%, at least 90%, at least 95%, at least 99%, at least 99.5%, at least 99.9%, at least 99.95%, at least 99.99%, at least 99.995%, at least 99.999%, or greater.

In some embodiments, the assay may include screening the at least one selected compound with a non-human animal capable of inducible expression of a mutant POLG1 polypeptide, as described herein. In some embodiments, the assay may include screening the at least one selected compound to measure an effect on protein or gene expression of a mitochondrial health biomarker, as identified herein. The method of screening may be repeated by executing the deep neural network to identify one or more compounds having a similarity score of at least 70% with at least one identified compound and/or at least one selected compound. In some embodiments, the deep neural network may be retrained with at least one identified compound and/or at least one selected compound.

In one embodiment, such a method of screening comprises the steps of: a) providing a non-human animal capable of inducible expression of a mutant POLG1 polypeptide; b) stimulating the expression of the mutant POLG1 polypeptide, wherein stimulating expression of the mutant POLG1 polypeptide induces a physiological or phenotypic response; c) administering an agent to the non-human animal either before step b) or after step b); d) determining the effect of the agent on pathology; and e) comparing the effect of the agent to a control animal, wherein a reduction or an increase (as appropriate) in the physiological or phenotypic response in the non-human animal after administration of the agent indicates the agent is a therapeutic agent for the treatment of the physiological or phenotypic response.

In another embodiment, the disclosure provides a method for identifying a therapeutic agent for the treatment of mitochondrial dysfunction.

In another embodiment, the disclosure provides a method for identifying a therapeutic agent for the treatment of a disease or condition associated with mitochondrial dysfunction or a symptom thereof.

In another embodiment, the disclosure provides a method for identifying a therapeutic agent for the treatment of an aging-associated chronic disease related to mitochondrial dysfunction or a symptom thereof.

In another embodiment, the disclosure provides a method for identifying a therapeutic agent for improving or preventing a decrease or condition in energy level and/or vitality.

In another embodiment, the disclosure provides a method for identifying a therapeutic agent for treating or preventing ovarian diseases or conditions and symptoms thereof. In any of the described methods of screening, agents can include, but are not limited to, chemical compounds, pharmaceutical compositions, cosmetic composition, extracts, plant extracts, seaweed extracts, microbial extracts, biological compounds and compositions (e.g., proteins, DNA, RNA, siRNAs, vaccines and the like), and microorganisms. Further, the agent may be selected from a library, including a library of agents approved by a regulatory authority such as the FDA.

In any of the described methods of screening, any of the transgenic non-human animals of the disclosure may be used.

In any of the described methods of screening, step b) may be accomplished by providing an inducer compound to the transgenic non-human animal or withholding the inducer compound from the transgenic non-human animal.

In any of the described methods of screening, the agent is added before step b). In any of the described methods of screening, the agent is added after step b).

In any of the described methods of screening, the animal model is an animal model described in the preceding section. In any of the described methods of screening, the mutant POLG1 polypeptide may be any mutant POLG1 polypeptide described herein. In certain aspects, the mutant POLG1 polypeptide comprises a dominant negative mutation. In certain aspects, the mutant POLG1 polypeptide comprises a D1135A mutation.

### Methods of Treatment

In one embodiment, the disclosure provides a method for treating or preventing mitochondrial dysfunction in a subject, the method comprising administering to said subject an effective amount of a compound of the disclosure, or a pharmaceutically acceptable form thereof. In one embodiment, the disclosure provides a method for treating or preventing a disease or condition associated with mitochondrial dysfunction or a symptom thereof in a subject, the method comprising administering to said subject an effective amount of a compound of the disclosure, or a pharmaceutically acceptable form thereof.

The disclosure provides compounds and compositions effective to treat or prevent diseases and conditions related to mitochondrial dysfunction, including mtDNA depletion. Diseases and conditions related to mitochondrial dysfunction include ovarian diseases or conditions and symptoms thereof. Ovarian diseases or conditions that may be treated by the methods disclosed herein include, for example, perimenopause, menopause, endometriosis, ovarian cysts, pre-menopausal or post-menopausal ovarian cancer, e.g., ovarian epithelial cancer, ovarian tumors, e.g., ovarian germ cell tumor, ovarian low malignant potential tumors, and ovarian stromal tumors, polycystic ovary syndrome (PCOS), primary ovarian insufficiency (POI), and ovarian torsion. The ovarian diseases or conditions may be associated with infertility, premature ovarian aging, and reduced oocyte mitochondrial mass. Treatment may include reducing or lessening the severity or frequency of symptoms of ovarian diseases or conditions including, for example, skin or vaginal dryness, pelvic pain or cramping, inflammation, prolonged or irregular menstrual cycles, and decreased ovulation events. The treatment or prevention may involve inducing mitochondrial biogenesis and/or improving mitochondrial function.

Thus, in certain embodiments, the disclosure provides compounds and compositions effective to treat or prevent, for example, reduce, inhibit, delay, and/or reverse, perimenopause, menopause, and/or symptoms thereof. Perimenopause is the stage before menopause, or the final period. Perimenopause may be characterized by altered levels of hormones, such as reduced estrogen and/or increased follicle stimulating hormone (FSH), reduced ovulation, and prolonged or irregular menstrual cycles. In certain instances, perimenopause may be characterized by FSH levels of about 20 IU/mL-30 IU/mL or greater in a blood sample and menopause may characterized by FSH levels of about 25 IU/mL-135 IU/mL or greater in a blood sample. However, in general, any altered level of a hormone may be compared to a baseline level of the subject to determine whether the subject is experiencing perimenopause or post-menopause. Symptoms of perimenopause and menopause include mood changes, changes in sexual desire, trouble concentrating, headaches, hot flashes, night sweats, vaginal dryness, trouble sleeping, joint or muscle aches, heavy sweating, frequent urination, and other pre-menstrual cycle (PMS)-like symptoms.

In certain embodiments, the ovarian diseases or conditions and symptoms thereof include those associated with mitochondrial dysfunction. While not being bound to any particular theory, the compounds disclosed herein may exert observed effects through inhibiting a decrease in mitochondrial DNA copy number or concentration, inhibiting depletion of mitochondrial DNA, inhibiting degradation of mitochondrial DNA, contributing to an increase in mitochondrial DNA copy number or concentration, repleting mitochondrial DNA, and/or promoting increased activity of mitochondrial DNA.

The methods disclosed herein relate to enhancing or improving fertility in a subject. Any reduced fertility of the subject may be age-related or premature. For example, the diseases, conditions, or symptoms disclosed herein may be associated with age-related ovarian aging or premature ovarian aging. The methods disclosed herein relate to extending reproductive longevity of a subject.

In accordance with certain embodiments, the methods disclosed herein relate to increasing oocyte mitochondrial mass or preventing a decrease of oocyte mitochondrial mass of a subject.

Certain extracts have been identified as comprising one or more compounds that promote and/or inhibit mtDNA. Emblica extract, Fucus extract, and chebula extract are described herein. It should be understood that similar extracts, in particular, extracts comprising one or more compounds disclosed herein, compounds that are constituents of an extract disclosed herein, and/or compounds having a similarity score of at least 95% with one or more compounds disclosed herein, are expected to provide similar mtDNA promotion and/or inhibition. Accordingly, other extracts, compounds derived from, constituents of, or purified from other extracts, and compounds having a similarity score of at least 95% with compounds derived from, constituents of, or purified from other extracts are within the scope of the disclosure.

Exemplary extracts include Polygonum aviculare extract, Physalis gngulata extract, Dunaliella salina extract, Camellia sinensis leaf extract, Tremella fuciformis sporocarp extract. Alteromonas ferment extract, Theobroma cacao (cocoa) seed extract, Vitis vinifera (grape) flower cell extract, Mirabilis jalapa callus extract, Alteromonas ferment extract, and Vibrio alginolyticus ferment filtrate.

Accordingly, the compounds described herein may be derived from, purified from, or isolated from the extract. The compounds described herein may be derived from, purified from, or isolated from a source other than the extract. A compound constituent of an extract may be derived from, purified from, or isolated from another natural or artificial source. In other embodiments, the compounds described herein may be synthetic. For instance, the compounds of the disclosure may be synthesized in a laboratory, manufacturing, or other setting. The above applies to compounds contained in or constituents of an extract, as well as compounds having a high similarity score thereof.

The methods disclosed herein may comprise administering to said subject an effective amount of an Emblica extract, or a pharmaceutically acceptable form thereof.

The methods disclosed herein may comprise administering to said subject an effective amount of a compound derived from, constituent of, or purified from an Emblica extract, or a pharmaceutically acceptable form thereof, or a compound having a similarity score of at least 95% with the compound derived from, constituent of, or purified from an Emblica extract, or a pharmaceutically acceptable form thereof.

The methods disclosed herein may comprise administering to said subject an effective amount of a Fucus extract, or a pharmaceutically acceptable form thereof.

The methods disclosed herein may comprise administering to said subject an effective amount of a compound derived from, constituent of, or purified from a Fucus extract, or a pharmaceutically acceptable form thereof, or a compound having a similarity score of at least 95% with the compound derived from, constituent of, or purified from a Fucus extract, or a pharmaceutically acceptable form thereof.

The methods disclosed herein may comprise administering to said subject an effective amount of a chebula extract, or a pharmaceutically acceptable form thereof.

The methods disclosed herein may comprise administering to said subject an effective amount of a compound derived from, constituent of, or purified from a chebula extract, or a pharmaceutically acceptable form thereof, or a compound having a similarity score of at least 95% with the compound derived from, constituent of, or purified from a chebula extract, or a pharmaceutically acceptable form thereof.

The disclosure may generally be related to extracts and compounds derived from, constituents of, or purified from extracts. It should be understood that compounds having a similarity score of at least 95%, for example, at least 96%, at least 97%, at least 98%, at least 99%, at least 99.5%, at least 99.9%, at least 99.95%, at least 99.99%, at least 99.995%, or at least 99.999% with a compound derived from, constituents of, or purified from the extract may be utilized in any composition as described herein.

Without wishing to be bound by theory, it is believed the administration of the compositions disclosed herein may involve inhibition of a decrease in mitochondrial DNA copy number or concentration, inhibition of a depletion of mitochondrial DNA, inhibition of a degradation of mitochondrial DNA, or a combination of the foregoing. Without wishing to be bound by theory, it is believed the administration of the compositions disclosed herein may involve inducing mitochondrial biogenesis and/or improving mitochondrial function.

In accordance with certain aspects, administration of the compositions disclosed herein may involve an increase in expression of mitochondrial oxidative phosphorylation complexes, an increase in stability of mitochondrial oxidative phosphorylation complexes, and/or an increase in expression of at least one protein selected from PGC-1a, TFAM, NRF-1, and COXII.

In accordance with certain aspects, administration of the compositions disclosed herein may involve modulation of a menstrual cycle, for example, normalizing a menstrual cycle, for example, regulating an amount and/or frequency of menstrual cycles. Administration of the compositions disclosed herein may involve an increase of ovulation events. Administration of the compositions disclosed herein may involve an increase of an anti-Mullerian hormone (AMH) level of the subject. Furthermore, administration of the compositions disclosed herein may decrease the probability of embryonic aneuploidy and/or Leigh's Syndrome.

In certain aspects, the Emblica extract is derived from Emblica officinalis (also known as Phyllanthus Emblica. Indian gooseberry, or by its Hindi name Amla). Such an extract from Emblica officinalis may be derived from any portion of the plant as desired. For example, the extract may be derived from the stem portion, the fruit portion, or both the stem portion and the fruit portion of Emblica officinalis. In preparing such an extract, the Emblica officinalis may be provided in a powdered from and extracted using chemical solvents known in the art, such as, but not limited to, aqueous ethyl acetate ethanol, and methanol. Other solvents or excipients disclosed herein may be used. In certain exemplary embodiments, the chemical solvent may be methanol.

In certain aspects, the effective amount of an Emblica extract or a compound derived from, constituent of, or purified from an Emblica extract is from 1 to 100 mg, such as 1 mg, 5, mg, 10 mg, 20 mg, 30 mg, 40 mg, 50 mg, 60 mg, 70 mg, 80 mg, 90 mg, or 100 mg.

In certain aspects, the compound derived from, constituent of, or purified from an Emblica extract is a breakdown product of a tannin, wherein the Emblica extract is optionally as described above. In certain aspects, the compound derived from, constituent of, or purified from an Emblica extract is an ellagitannin. In certain aspects, the compound derived from, constituent of, or purified from an Emblica extract is Emblicanin A, Emblicanin B, punigluconin pedunculagin, and/or chebulinic acid.

In certain aspects, the compound is an ellagitannin or a compound having a similarity score of at least 95% with an ellagitannin. Exemplary ellagitannins and compounds having a high similarity score with such ellagitannins are listed in Table 1. Other compounds having a high similarity score with ellagitannins are within the scope of the disclosure.

TABLE 1
Ellagitannins and compounds having a high similarity score with ellagitannins

| Ellagitannin | Compound having high similarity score with ellagitannin |
|---|---|
| Emblicanin b | Eugeniin |
| Emblicanin a | 1,2,3,4-Tetrakis-O-Galloyl-Alpha-D-Glucose |
| Emblicanin a | 1,2,3,6-Tetragalloylglucose |
| Emblicanin b | Punicafolin |
| Emblicanin a | 4-hydroxy-3,5-bis(3,4,5-trihydroxybenzoyloxy)-6-[(3,4,5- |
| | trihydroxybenzoyloxy)methylloxan-2-yl 3,4,5-trihydroxybenzoate |
| Emblicanin b | Punicalin |
| Emblicanin a | Thonningianin A |
| Emblicanin a | Sagerinic Acid |
| Emblicanin b | Casuariin |
| Emblicanin a | Prunasin 2′,3′,4′,6′-Tetra-O-Gallate |
| Emblicanin b | Epigallocatechin-(4Beta -> 8)-Epigallocatechin-3-O-Gallate |
| Emblicanin b | Arjuna |
| Emblicanin a | Hyemaloside A |
| Emblicanin b | Trapain |
| Emblicanin a | 4-[3,4-dihydroxy-5-(3,4,5-trihydroxybenzoyloxy)benzoyloxy]-1- |
| | hydroxy-3,5-bis(3,4,5-trihydroxybenzoyloxy)cyclohexane-1- |
| | carboxylic acid |
| Emblicanin a | Pentagalloylglucose |
| Emblicanin a | Myricetin 3-(2″,3″-Digalloylrhamnoside) |
| Emblicanin b | Trilobatin G |
| Emblicanin b | Stachyurin |
| Emblicanin a | Eutannin |
| Emblicanin b | Thonningianin A |
| Emblicanin b | Castalagin |
| Emblicanin b | Casuarictin |
| Emblicanin a | Yunnaneic Acid G |
| Emblicanin a | Rabdosiin |
| Emblicanin b | 2-(3,4-dihydroxyphenyl)-5,7-dihydroxy-8-[3,5,7-trihydroxy-2- |
| | (3,4,5-trihydroxyphenyl)-3,4-dihydro-2H-1-benzopyran-4-yl]-3,4- |
| | dihydro-2H-1-benzopyran-3-yl 3,4,5-trihydroxybenzoate |
| Emblicanin b | Pterocaryanin C |
| Emblicanin b | Tercatain |
| Emblicanin a | Trapain |
| Emblicanin b | Tellimagrandin I |
| Emblicanin b | Sagerinic Acid |
| Emblicanin b | Paeonianin E |
| Emblicanin a | 2-(3-{[1-carboxy-2-(3,4-dihydroxyphenyl)ethoxy]carbonyl}-2- |
| | (3,4-dihydroxyphenyl)-7,8-dihydroxy-1,2-dihydronaphthalene-1- |
| | carbonyloxy)-3-(3,4-dihydroxyphenyl)propanoic acid |
| Emblicanin a | 2-{[2-(3,4-dihydroxyphenyl)-5,7-dihydroxy-4-oxo-4H-chromen- |
| | 3-yl]oxy}-4,5-dihydroxy-6-[(3,4,5-trihydroxybenzoyloxy)methyl] |
| | oxan-3-yl 3,4,5-trihydroxybenzoate |
| Emblicanin a | Remurin B |
| Emblicanin a | Arjuna |
| Emblicanin a | 5-Hydroxyferuloyl-Coa |
| Emblicanin a | Geraniinic Acid B |
| Emblicanin a | Casuariin |
| Emblicanin a | Eugeniin |
| Emblicanin a | Sinapoyl-Coa |
| Emblicanin b | Geraniin |
| Emblicanin a | Feruloyl-Coa |
| Emblicanin a | Balanophotannin A |
| Emblicanin b | 6-(4-{7-[(6-carboxy-3,4,5-trihydroxyoxan-2-yl)oxy]-5-hydroxy-4- |
| | oxo-4H-chromen-2-yl}phenoxy)-5-[(6-carboxy-4,5-dihydroxy-3- |
| | {[3-(4-hydroxyphenyl)prop-2-enoyl]oxy}oxan-2-yl)oxy]-3,4- |
| | dihydroxyoxane-2-carboxylic acid |
| Emblicanin a | Chebulagic Acid |
| Emblicanin b | Eutannin |
| Emblicanin b | Balanophotannin A |
| Emblicanin b | Myricetin 3-(2″,3″-Digalloylrhamnoside) |
| Emblicanin b | Pradimicin C |
| Emblicanin b | 5,5″-Bis-[2,3-Dicarboxy-6,7-Dihydroxy-1-(3′,4′- |
| | Dihydroxyphenyl)-1,2-Dihydronaphthalene] |
| Emblicanin a | Stachyurin |
| Emblicanin a | Punicafolin |
| Emblicanin b | Alnusiin |
| Emblicanin b | Phyllanthusiin D |
| Emblicanin a | Caffeoyl-Coa |

In certain aspects, the compound is chebulinic acid or a compound having a similarity score of at least 95% with chebulinic acid (formula I below). Exemplary compounds having a high similarity score with chebulinic acid are listed in Table 2.

TABLE 2
Compounds having a high similarity score with chebulinic acid.

| Compound ID | |
|---|---|
| from COCONUT | |
| Database | Structure |
| CNP0285899 | 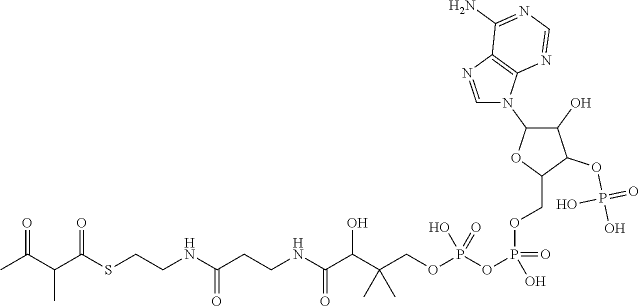 [SMILES: CC(=O)C(C)C(=O)SCCNC(=O)CCNC(=O)C(O)C(C)(C)COP(=O)(O)OP(=O)(O)OCC1OC(n2cnc3c(N)ncnc32)C(O)C1OP(=O)(O)O] |
| CNP0059074 | 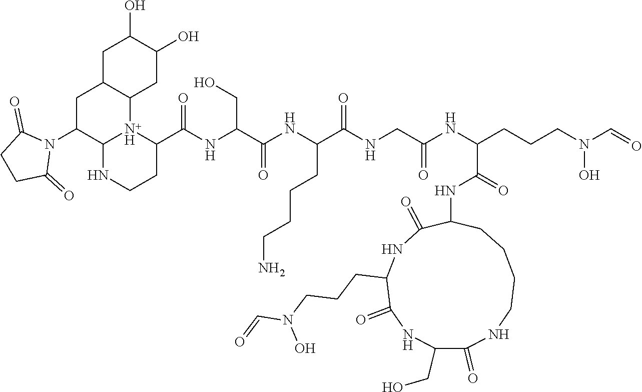 [SMILES: NCCCCC(NC(=O)C(CO)NC(=O)C1CCNC2C(N3C(=O)CCC3=O)CC3CC(O)C(O)CC3N12)C(=O)NCC(=O)NC(CCCN(O)C=O)C(=O)NC1CCCCNC(=O)C(CO)NC(=O)C(CCCN(O)C=O)NC1=O] |
| CNP0343660 | 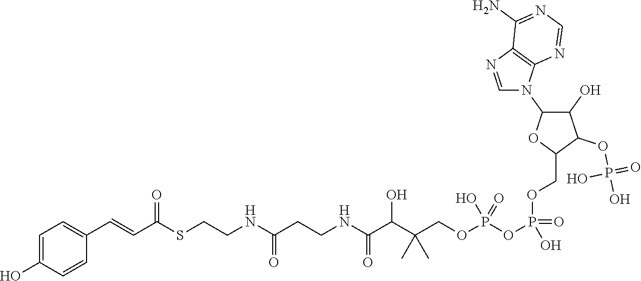 [SMILES: CC(C)(COP(=O)(O)OP(=O)(O)OCC1OC(n2cnc3c(N)ncnc32)C(O)C1OP(=O)(O)O)C(O)C(=O)NCCC(=O)NCCSC(=O)/C=C/c1ccc(O)cc1] |
| CNP0139083 | 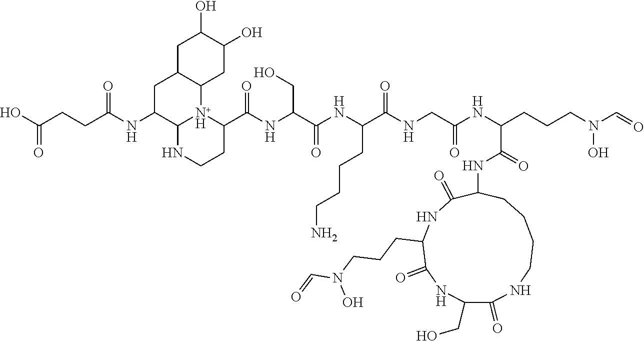 [SMILES: NCCCCC(NC(=O)C(CO)NC(=O)C1CCNC2C(NC(=O)CCC(=O)O)CC3CC(O)C(O)CC3N12)C(=O)NCC(=O)NC(CCCN(O)C=O)C(=O)NC1CCCCNC(=O)C(CO)NC(=O)C(CCCN(O)C=O)NC1=O] |
| CNP0041318 | 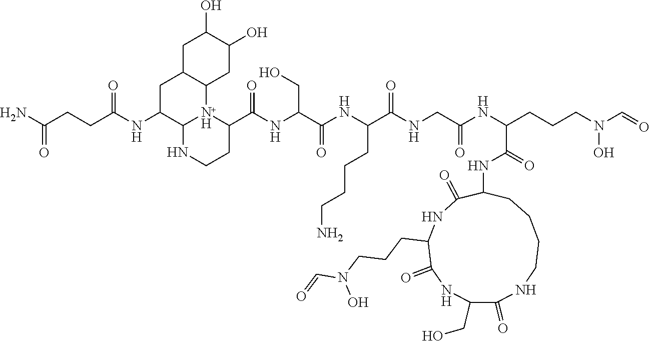 [SMILES: NCCCCC(NC(=O)C(CO)NC(=O)C1CCNC2C(NC(=O)CCC(N)=O)CC3CC(O)C(O)CC3N12)C(=O)NCC(=O)NC(CCCN(O)C=O)C(=O)NC1CCCCNC(=O)C(CO)NC(=O)C(CCCN(O)C=O)NC1=O] |
| CNP0422373 | 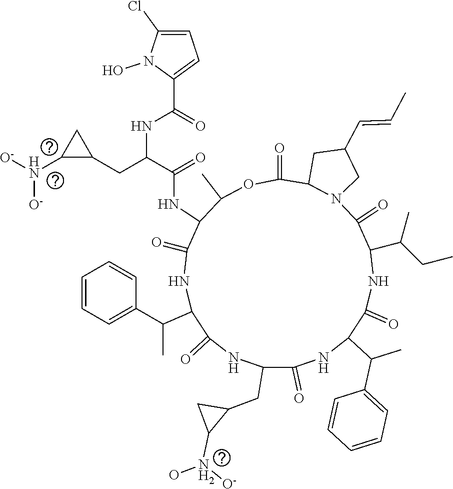 |
| CNP0415802 | 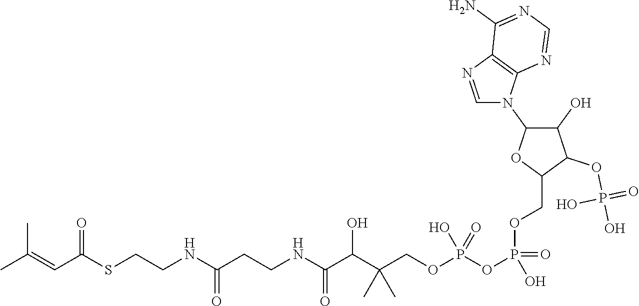 [SMILES: CC(C)=CC(=O)SCCNC(=O)CCNC(=O)C(O)C(C)(C)COP(=O)(O)OP(=O)(O)OCC1OC(n2cnc3c(N)ncnc32)C(O)C1OP(=O)(O)O] |
| CNP0070782 | 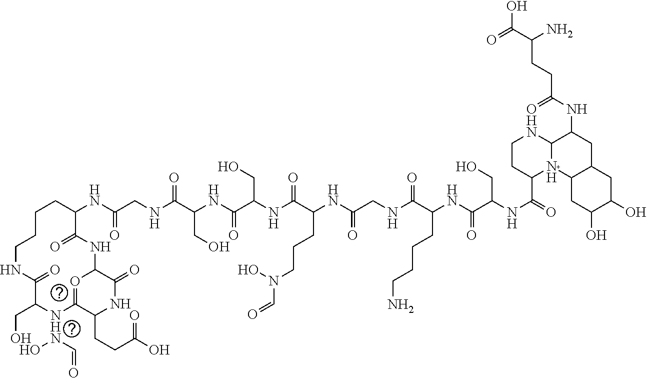 |
| CNP0102046 | 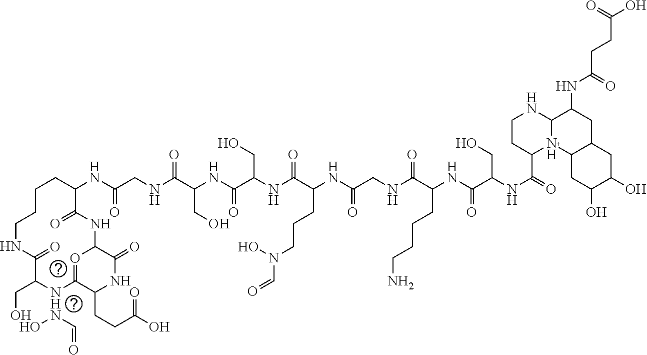 |
| CNP0098207 | 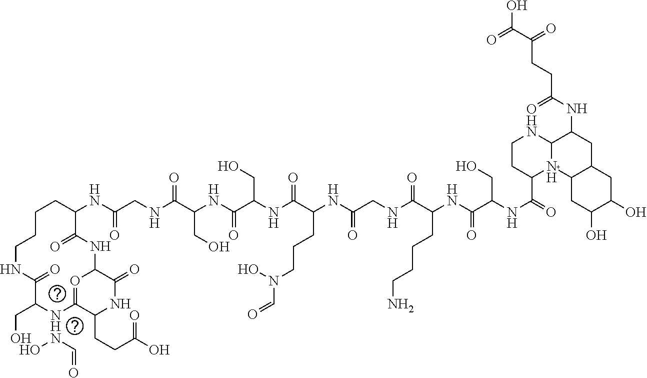 |
| CNP0026197 | 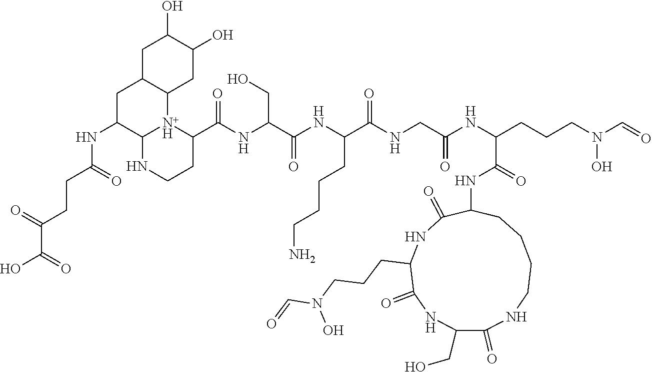 [SMILES: NCCCCC(NC(=O)C(CO)NC(=O)C1CCNC2C(NC(=O)CCC(=O)C(=O)O)CC3CC(O)C(O)CC3N12)C(=O)NCC(=O)NC(CCCN(O)C=O)C(=O)NC1CCCCNC(=O)C(CO)NC(=O)C(CCCN(O)C=O)NC1=O] |
| CNP0326396 | 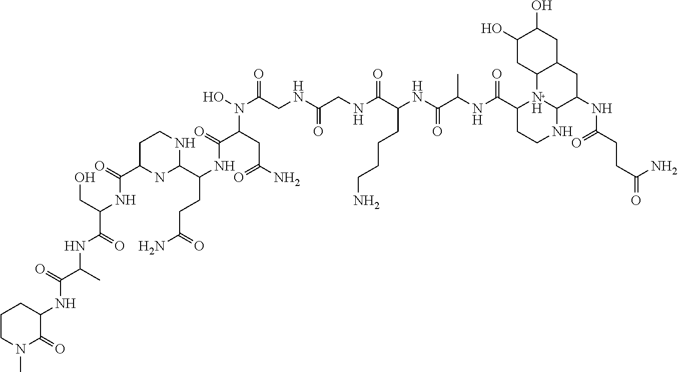 [SMILES: CC(NC(=O)C(CO)NC(=O)C1CCNC(C(CCC(N)=O)NC(=O)C(CC(N)=O)N(O)C(=O)CNC(=O)CNC(=O)C(CCCCN)NC(=O)C(C)NC(=O)C2CCNC3C(NC(=O)CCC(N)=O)CC4CC(O)C(O)CC4N23)N1)C(=O)NC1CCCN(C)C1=O] |
| CNP0384812 | 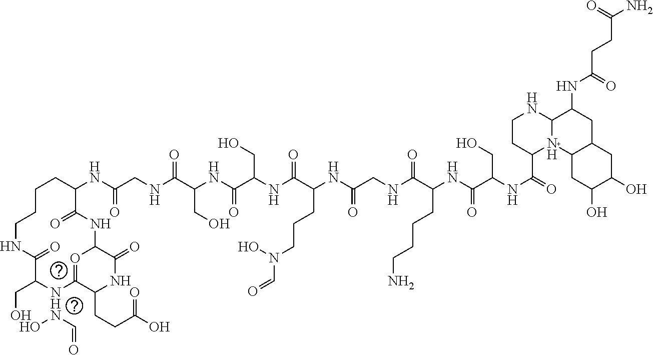 |
| CNP0024232 | 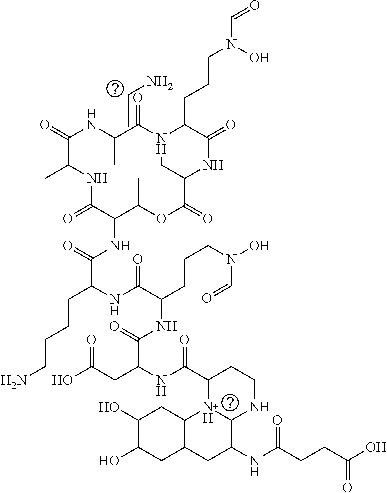 |
| CNP0070582 | 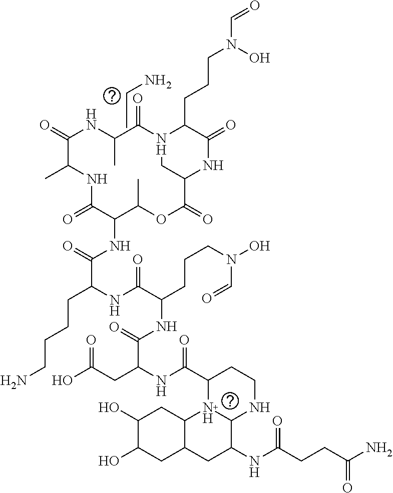 |

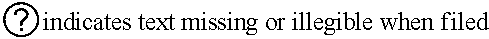

In certain aspects, the compound derived from, constituent of, or purified from an Emblica extract is a benzoic acid substituted with 1 to 5 hydroxy groups and optionally 1 to 3 O—(C1-C5 alkyl) or O—(C1-C5 alkenyl) groups. In certain aspects, the compound derived from, constituent of, or purified from an Emblica extract is a benzoic acid substituted with 1 to 3 hydroxy groups and optionally 1 to 2 O—(C1-C5 alkyl) or O—(C1-C5 alkenyl) groups.

In certain aspects, the compound derived from, constituent of, or purified from an Emblica extract is a benzene substituted with —CH═CH—(CH₂)ₐ—C(O)OH, wherein a is 0 to 5, and 1 to 5 hydroxy groups. In certain aspects, the compound derived from, constituent of, or purified from an Emblica extract is a benzene substituted with —CH═CH—(CH₂)ₐ—C(O)OH, wherein a is 0 to 5, and 1 to 3 hydroxy groups. In certain aspects, the compound derived from, constituent of, or purified from an Emblica extract is a benzene substituted with —CH═CH—(CH₂)ₐ—C(O)OH, wherein a is 0, and 1 to 3 hydroxy groups.

In certain aspects, the compound derived from, contained in, or purified from an Emblica extract is gallic acid, vanillic acid, chlorogenic acid, caffeic acid, syringic acid, coumaric aid, quercetin, Emblicanin A, Emblicanin B, punigluconin, and pedunculagin, punicafolin, phyllanemblin, kaempferol, ellagic acid, chebulinic acid, chebulagic acid, punicalagin, or a metabolite of any of the foregoing.

In certain aspects, the compound derived from, constituent of, or purified from an Emblica extract is ascorbic acid or citric acid.

In certain aspects, the compound derived from, constituent of, or purified from an Emblica extract is gallic acid, vanillic acid, chlorogenic acid, caffeic acid, syringic acid, coumaric aid, quercetin, vitamin C, or a metabolite of any of the foregoing.

In certain aspects, the compound derived from, constituent of, or purified from an Emblica extract is gallic acid or a compound having a similarity score of at least 95% with gallic acid.

In certain aspects, the active agent in the composition is gallic acid. The active agent may be or comprise an ellagitannin. The active agent may be or comprise Emblicanin A, Emblicanin B, punigluconin, pedunculagin, and/or chebulinic acid.

In accordance with certain embodiments, the composition may comprise an Emblica extract fortified with one or more compound that is a constituent of an Emblica extract. The compound constituent of the Emblica extract or combination of compounds constituents of the Emblica extract may be purified, e.g., at least 80% purified, at least 85% purified, at least 90% purified, at least 95% purified, at least 98% purified, at least 99% purified, at least 99.9% purified, at least 99.99% purified, or at least 99.999% purified.

In certain aspects, the effective amount of a compound derived from, constituent of, or purified from an Emblica extract, optionally the effective amount of an active agent of the composition, is from 1 to 100 mg of the, such as 1 mg, 5 mg, 10 mg, 20 mg, 30 mg, 40 mg, 50 mg, 60 mg, 70 mg, 80 mg, 90 mg, or 100 mg of the compound derived from, constituent of, or purified from an Emblica extract.

In certain aspects, the Fucus extract is derived from Fucus vesiculosus, Fucus serratus, Fucus, spiralis, or Fucus guiryi. In certain aspects, the Fucus extract is derived from Fucus vesiculosus. Such an extract may be derived from any portion of the algae as desired. In preparing such an extract, the Fucus vesiculosus, Fucus serratus, Fucus, spiralis, or Fucus guiryi may be provided in a powdered from and extracted using chemical solvents known in the art, such as, but not limited to, aqueous ethyl acetate ethanol, and methanol. Other solvents or excipients disclosed herein may be used. In certain exemplary embodiments, the chemical solvent may be methanol.

In certain aspects, the effective amount of Fucus extract is from 1 to 100 mg of extract, such as 1 mg, 5 mg, 10 mg, 20 mg, 30 mg, 40 mg, 50 mg, 60 mg, 70 mg, 80 mg, 90 mg, or 100 mg of a Fucus extract.

In certain aspects, the compound derived from, constituent of, or purified from a Fucus extract is a benzoic acid substituted with 1 to 5 hydroxy groups and optionally 1 to 3 O—(C1-C5 alkyl) or O—(C₁-C₅ alkenyl) groups. In certain aspects, the compound derived from, constituent of, or purified from a Fucus extract is a benzoic acid substituted with 1 to 3 hydroxy groups and optionally 1 to 2 O—(C₁-C₅ alkyl) or O—(C₁-C₅ alkenyl) groups.

In certain aspects, the compound derived from, constituent of, or purified from a Fucus extract is a benzene substituted with —CH═CH—(CH₂)ₐ—C(O)OH, wherein a is 0 to 5, and 1 to 5 hydroxy groups. In certain aspects, the compound derived from, constituent of, or purified from a Fucus extract a benzene substituted with —CH—CH—(CH₂)ₐ—C(O)OH, wherein a is 0 to 5, and 1 to 3 hydroxy groups. In certain aspects, the compound derived from, constituent of, or purified from a Fucus extract a benzene substituted with —CH—CH—(CH₂)ₐ—C(O)OH, wherein a is 0, and 1 to 3 hydroxy groups.

In certain aspects, the compound derived from, constituent of, or purified from a Fucus extract is gallic acid, vanillic acid, chlorogenic acid, caffeic acid, syringic acid, coumaric aid, or a metabolite of any of the foregoing.

In certain aspects, the compound derived from, constituent of, or purified from a Fucus extract is gallic acid.

In certain aspects, the active agent in the composition is gallic acid or a compound having a similarity score of at least 95% with gallic acid. In certain aspects, the active agent in the composition is chebulinic acid or a compound having a similarity score of at least 95% with chebulinic acid (see, e.g., Table 2 above). The active agent may be or comprise an ellagitannin or a compound having a similarity score of at least 95% with an ellagitannin (see, e.g., Table 1 above). The active agent may be or comprise fucoidan or a compound having a similarity score of at least 95% with fucoidan.

In accordance with certain embodiments, the composition may comprise a Fucus extract fortified with one or more compound that is a constituent of a Fucus extract. The compound constituent of a Fucus extract or combination of compounds constituents of a Fucus extract may be purified, e.g., at least 80% purified, at least 85% purified, at least 90% purified, at least 95% purified, at least 98% purified, at least 99% purified, at least 99.9% purified, at least 99.99% purified, or at least 99.999% purified.

In certain aspects, the effective amount of a compound derived from, constituent of, or purified from a Fucus extract, optionally the effective amount of an active agent of the composition, is from 1 to 100 mg, such as 1 mg, 5 mg, 10 mg, 20 mg, 30 mg, 40 mg, 50 mg, 60 mg, 70 mg, 80 mg, 90 mg, or 100 mg of the compound derived from, constituent of, or purified from a Fucus extract.

In certain aspects, the chebula extract is derived from Terminilia chebula, Terminalia arborea, or Lumnitzera racemose. In certain aspects, the chebula extract is derived from Termibnilia chebula. Such an extract may be derived from any portion of the plant as desired. In preparing such an extract, the Terminilia chebula, Terminalia arborea, or Lumnitzera racemose may be provided in a powdered from and extracted using chemical solvents known in the art, such as, but not limited to, aqueous ethyl acetate ethanol, and methanol. Other solvents or excipients disclosed herein may be used. In certain exemplary embodiments, the chemical solvent may be methanol.

In certain aspects, the effective amount of chebula extract is from 1 to 100 mg of extract, such as 1 mg, 5 mg, 10 mg, 20 mg, 30 mg, 40 mg, 50 mg, 60 mg, 70 mg, 80 mg, 90 mg, or 100 mg of a chebula extract.

In certain aspects, the compound derived from, constituent of, or purified from a chebula extract is a breakdown product of a tannin. In certain aspects, the compound derived from, constituent of, or purified from a chebula extract is an ellagitannin. In certain aspects, the compound derived from, constituent of, or purified from a chebula extract is chebulinic acid.

In certain aspects, the compound derived from, constituent of, or purified from a chebula extract is a breakdown product of a tannin. In certain aspects, the compound derived from, constituent of, or purified from a Fucus extract is an ellagitannin. In certain aspects, the compound derived from, constituent of, or purified from a Fucus extract is fucoidan.

In certain aspects, the compound derived from, constituent of, or purified from a chebula extract is a benzoic acid substituted with 1 to 5 hydroxy groups and optionally 1 to 3 O—(C₁-C₅ alkyl) or O—(C₁-C₅ alkenyl) groups. In certain aspects, the compound derived from, contained in, or purified from a Fucus extract is a benzoic acid substituted with 1 to 3 hydroxy groups and optionally 1 to 2 O—(C₁-C₅ alkyl) or O—(C₁-C₅ alkenyl) groups.

In certain aspects, the compound derived from, constituent of, or purified from a chebula extract is a benzene substituted with —CH═CH—(CH₂)ₐ—C(O)OH, wherein a is 0 to 5, and 1 to 5 hydroxy groups. In certain aspects, the compound derived from, constituent of, or purified from a chebula extract a benzene substituted with —CH═CH—(CH₂)ₐ—C(O)OH, wherein a is 0 to 5, and 1 to 3 hydroxy groups. In certain aspects, the compound derived from, constituent of, or purified from a chebula extract a benzene substituted with —CH═CH—(CH₂)ₐ—C(O)OH, wherein a is 0, and 1 to 3 hydroxy groups.

In certain aspects, the compound derived from, constituent of, or purified from a chebula extract is gallic acid, vanillic acid, chlorogenic acid, caffeic acid, syringic acid, coumaric aid, or a metabolite of any of the foregoing.

In certain aspects, the compound derived from, constituent of, or purified from a chebula extract is chebulinic acid.

In certain aspects, the active agent in the composition is chebulinic acid or a compound having a similarity score of at least 95% with chebulinic acid (see, e.g., Table 2 above). The active agent may be or comprise an ellagitannin or a compound having a similarity score of at least 95% with an ellagitannin (see, e.g., Table 1 above).

In accordance with certain embodiments, the composition may comprise a chebula extract fortified with one or more compound that is a constituent of a chebula extract. The compound constituent of a chebula extract or combination of compounds constituents of a chebula extract may be purified, e.g., at least 80% purified, at least 85% purified, at least 90% purified, at least 95% purified, at least 98% purified, at least 99% purified, at least 99.9% purified, at least 99.99% purified, or at least 99.999% purified.

In certain aspects, the effective amount of a compound derived from, constituent of, or purified from a chebula extract, optionally the effective amount of an active agent of the composition, is from 1 to 100 mg, such as 1 mg, 5 mg, 10 mg, 20 mg, 30 mg, 40 mg, 50 mg, 60 mg. 70 mg. 80 mg, 90 mg, or 100 mg of the compound derived from, constituent of, or purified from a chebula extract.

The methods, and any of aspects of the foregoing, may comprise administering a composition comprising an effective amount of two or more of an Emblica extract or a compound constituent of an Emblica extract, an effective amount of a Fucus extract or a compound constituent of a Fucus extract, and an effective amount of a chebula extract or a compound constituent of a chebula extract. In accordance with certain embodiments, the two or more of the Emblica extract or compound constituent of the Emblica extract, the Fucus extract or compound constituent of the Fucus extract, and the chebula extract or compound constituent of the chebula extract may provide synergistic effects in the treatment of the disease or condition.

The compositions disclosed herein may comprise or be fortified with one or more of: an Emblica extract or compound constituent of an Emblica extract, a Fucus extract or compound constituent of a Fucus extract, a chebula extract or compound constituent of the chebula extract, BAMLET 10, BAMLET 50, ferulic acid, quercetin, urolithin A, pterostilbene, acadesine, embelin, EGCG, criocitrin, gallic acid, gomsin A, lutein, luteolin, NAD, rutin, zeaxanthin, and melatonin.

The compositions disclosed herein may be administered in combination with and/or encapsulated in a biomaterial. Biomaterials provide certain advantages for drug delivery, including promoting favorable cellular interaction, relatively high drug loading content, controllable drug release, excellent passive and active targeting, biocompatibility and low toxicity. Biomaterials can be classified into three basic categories: 1) natural biomaterials (e.g., extracellular vesicles, collagen, hyaluronic acid, fibrin), 2) synthetic biomaterials (e.g., polylactic acid (PLA), polyglycolic acid (PGA), polycaprolactone (PCL), polyethylene glycol (PEG)), and 3) composite biomaterials (e.g., protein-polysaccharide composite biomaterials, nanocomposite biomaterials, sponges).

Biomaterials may be used to encapsulate and deliver the compounds disclosed herein, and optional combination therapies, for treatment of ovarian diseases and conditions. In accordance with certain embodiments, the biomaterials described herein may be nontoxic, biocompatible, biodegradable, bioresorbable, and/or able to support cellular and tissue regeneration without a resultant inflammatory reaction. Biomaterials may be used in connection with artificial ovary construction, follicle development, biomaterial encapsulation of cells and/or compositions, and the delivery of natural extracellular vesicles. Exemplary biomaterials include extracellular vesicles, collagen, hyaluronic acid, synthetic biomaterials, fibrin, and alginate.

Extracellular vesicles (EVs) are a heterogeneous group of cell-derived membranous structures comprising of exosomes (˜50-150 nm) and microvesicles (˜100-1000 nm), which carry bioactive material and protein in different body fluids and deliver their contents to recipient cells. EVs may be employed as effective nanocarriers of the compositions disclosed herein for advanced drug delivery due to multiple advantages including good biocompatibility with the immune system and low toxicity. Stem cell-derived EVs may be employed to transfer functional miRNAs and proteins to ovarian tissues. Additionally, EVs may be readily isolated from stem cells of various origins and carry biologically active molecules that can be transferred to target ovarian cells to exert their therapeutic effects.

Collagen is the most abundant extracellular matrix protein in the animal kingdom, and it transmits loads in tissues and provides a highly biocompatible environment for cells. Collagen may be used as a scaffold for stem cell-based ovarian therapy. Such scaffolds may be employed in connection with treatments for primary ovarian failure (POF) and increase the long-term retention of adipose-derived stem cells (ADSCs) and contribute to the restoration of ovarian function, including a regular estrus cycle, elevated E2 levels, improved fertility, and successful clinical pregnancy. Collagen may be used to improve targeted delivery of the compositions disclosed herein to ovarian tissues.

Hyaluronic acid (HA) is present in all vertebrates, is an important component of the ECM in most mature tissues, and plays a significant role in establishing a microenvironment conducive to the development of follicles in ovaries. HA has excellent physiochemical properties and may be used for drug delivery. Self-linked HA is a good cell scaffold to improve the transplantation of stem cells in the treatment of ovarian therapy. Additionally, HA may be used to improve targeted delivery of the compositions disclosed herein to ovarian tissues.

Fibrin (FIB) is a natural scaffold formed after tissue injury, and it has excellent biocompatibility, controllability, biodegradability, and the ability to transfer cells and biomolecules to a target site. Primate oocytes derived from primary follicles cultured in fibrin-alginate 3D capsules may be employed to restart meiosis for fertilization. Fibrin scaffolds may be used for follicular transplantation by controlling the release of growth factors and creating a continuous path of cell infiltration. Fibrin may be used to improve targeted delivery of the compositions disclosed herein to ovarian tissues.

Synthetic biomaterials may be designed, selected, and/or tailored according to the physicochemical and mechanical properties of target biological tissues. PEG is a commonly used biocompatible polymer, and PEG hydrogels have been shown to provide a good microenvironment for follicles. Synthetic biomaterials may be designed to deliver the compositions disclosed herein to target ovarian tissues.

Alginate is a group of non-branched polysaccharides that is non-toxic, nutrient-rich, biocompatible and biodegradable in humans. Alginate is able to form a soft hydrogel under physiological conditions, with pores large enough to allow nutrients and growth factors to pass through freely, while trapping cells in the polymer network. Alginate may be used to improve targeted delivery of the compositions disclosed herein to ovarian tissues.

The methods, and any aspects of the foregoing, may further comprise one or more of the steps: (i) identifying a subject in need of treatment or administration; and (ii) providing a compound of the disclosure or a pharmaceutical composition comprising a compound of the disclosure.

In any of the foregoing embodiments, and any aspects of the foregoing, when the term “preventing” is used the term may refer to at least a partial inhibition, for example a 10% inhibition, a 20% inhibition, a 30% inhibition, a 40% inhibition, 50% inhibition, a 60% inhibition, a 70% inhibition, an 80% inhibition, a 90% inhibition, a 95% inhibition or greater than 95% inhibition.

In any of the foregoing embodiments, and any aspects of the foregoing, when the term “improving” is used the term may refer to at least partial improvement, for example, a 10% improvement, a 20% improvement, a 30% improvement, a 40% improvement, 50% improvement, a 60% improvement, a 70% improvement, an 80% improvement, a 90% improvement, a 95% improvement or greater than 95% improvement.

In any of the foregoing embodiments, and any aspects of the foregoing, when the term “enhancing” is used the term may refer to at least partial enhancement, for example, a 10% enhancement, a 20% enhancement, a 30% enhancement, a 40% enhancement, 50% enhancement, a 60% enhancement, a 70% enhancement, an 80% enhancement, a 90% enhancement, a 95% enhancement or greater than 95% enhancement.

In any of the foregoing embodiments, and any aspects of the foregoing, when the term “increasing” is used the term may refer to at least partial increase, for example, a 10% increase, a 20% increase, a 30% increase, a 40% increase, 50% increase, a 60% increase, a 70% increase, an 80% increase, a 90% increase, a 95% increase or greater than 95% increase. The term may refer to a two-fold, three-fold, four-fold, five-fold, ten-fold, one hundred-fold increase, or more.

In any of the foregoing embodiments, and any aspects of the foregoing, the compound, or pharmaceutically acceptable form thereof, may be administered alone or as a part of a pharmaceutical composition. The pharmaceutical composition may be formulated by combining a solution of the compound with a pharmaceutically suitable carrier. The solution of the compound may comprise 1 to 1,000 mg of the compound, for example, 10 to 600 mg, 20 to 500 mg. 30 to 200 mg, 50 to 100 mg, or 1 mg, 5 mg, 10 mg, 20 mg, 30 mg, 40 mg, 50 mg, 60 mg, 70 mg, 80 mg, 90 mg, 100 mg, 200 mg, 300 mg, 400 mg. 500 mg, 600 mg, 700 mg, 800 mg, 900 mg, or 1,000 mg of the compound.

The compound and the pharmaceutically suitable carrier may be combined in a ratio of 1:5 to 5:1. The pharmaceutical composition may be formulated to have a concentration of 1 to 1,000 mg/ml of the compound, for example, 10 to 600 mg/ml, 20 to 500 mg/ml, 30 to 200 mg/ml, 50 to 100 mg/ml, or 1 mg/ml, 5 mg/ml, 10 mg/ml, 20 mg/ml, 30 mg/ml, 40 mg/ml, 50 mg/ml, 60 mg/ml, 70 mg/ml, 80 mg/ml, 90 mg/ml, 100 mg/ml, 200 mg/ml, 300 mg/ml, 400 mg/ml, 500 mg/ml, 600 mg/ml, 700 mg/ml, 800 mg/ml, 900 mg/ml, or 1,000 mg/ml of the compound.

The pharmaceutical composition may be formulated to have a concentration of 0.01% to 2% of the compound, for example, 0.01%, 0.02%, 0.05%, 0.1%, 0.2%, 0.5%, 1%, or 2% of the compound.

The pharmaceutical composition may be formulated to have a concentration of 2.5 to 50 μM of the compound, for example, 2.5 μM, 5 μM, 10 μM, 25 μM, or 50 μM of the compound. The pharmaceutical composition may be formulated to have a concentration of 50 to 500 μM of the compound, for example, 50 μM, 100 μM, 200 μM, 300 μM, 400 μM, or 500 UM of the compound.

The pharmaceutical composition may be formulated to have a concentration of 2.5 to 50 μM of the compound, for example, 2.5 μg/L, 5 μg/L, 10 μg/L, 25 μg/L, or 50 μg/L of the compound. The pharmaceutical composition may be formulated to have a concentration of 50 to 500 μg/L of the compound, for example, 50 μg/L, 100 μg/L, 200 μg/L, 300 μg/L, 400 μg/L, or 500 μg/L of the compound.

In any of the foregoing embodiments, and any aspects of the foregoing, the compound, or pharmaceutically acceptable form thereof, may be administered alone or as a part of a cosmetic composition. The cosmetic composition may be formulated by combining a solution of the compound with a cosmetically suitable carrier. The solution of the compound may comprise 1 to 1,000 mg of the compound, for example, 10 to 600 mg, 20 to 500 mg, 30 to 200 mg, 50 to 100 mg, or 1 mg, 5 mg, 10 mg, 20 mg. 30 mg, 40 mg, 50 mg, 60 mg, 70 mg, 80 mg, 90 mg, 100 mg, 200 mg. 300 mg, 400 mg, 500 mg, 600 mg, 700 mg, 800 mg, 900 mg, or 1,000 mg of the compound.

The compound and the cosmetically suitable carrier may be combined in a ratio of 1:5 to 5:1. The cosmetic composition may be formulated to have a concentration of 1 to 1,000 mg/ml of the compound, for example, 10 to 600 mg/ml, 20 to 500 mg/ml, 30 to 200 mg/ml, 50 to 100 mg/ml, or 1 mg/ml, 5 mg/ml, 10 mg/ml, 20 mg/ml, 30 mg/ml, 40 mg/ml, 50 mg/ml, 60 mg/ml, 70 mg/ml, 80 mg/ml, 90 mg/ml, 100 mg/ml, 200 mg/ml, 300 mg/ml, 400 mg/ml, 500 mg/ml, 600 mg/ml, 700 mg/ml, 800 mg/ml, 900 mg/ml, or 1,000 mg/ml of the compound.

In any of the methods, and any aspects of the foregoing, a compound described herein is in the form of a pharmaceutically acceptable salt, solvate, or hydrate. Such a compound may be formulated as a pharmaceutically acceptable salt, e.g., acid addition salt, and complexes thereof. The preparation of such salts can facilitate the pharmacological use by altering the physical characteristics of the agent without preventing its physiological effect. Examples of useful alterations in physical properties include, but are not limited to, increasing the solubility to facilitate administering higher concentrations of the compound.

In any of the methods, and any aspects of the foregoing, a compound or an extract described herein is administered topically, intravenously, intraperitoneally, parenterally, intramuscularly, orally or via the respiratory tract. In any of the methods, and any aspects of the foregoing, the compound or the extract is administered topically. In any of the methods, and any aspects of the foregoing, the compound or the extract is administered parenterally, e.g., by injection.

The compound or an extract described herein may be administered locally, e.g., at a local site of the disease or condition. The compound or an extract described herein may be formulated for local administration, e.g., to a target site of the disease or condition. The compound or an extract described herein may be administered systemically. Systemic administration may be, e.g., topical, intravenous, intraperitoneal, parenteral, intramuscular, oral, or via the respiratory tract. The compound or an extract described herein may be formulated for systemic administration.

In any of the methods, and any of the aspects of the foregoing, the subject animal is a vertebrate. In any of the methods, and any aspects of the foregoing, the subject is a mammal. In any of the methods, and any aspects of the foregoing, the subject is a human. In any of the methods, and any of aspects of the foregoing, the subject is a non-human mammal, e.g., a rodent, e.g., a mouse.

In some aspects, the subject may be female. In some aspects, the subject may be male. The subject may be characterized as one of the following ethnicity/race: Asian, Black or African American, Hispanic or Latino, white, or multi-racial. The subject may be of an age less than 1, or between 1-5, 5-10, 10-20, 20-30, 30-40, 40-50, 50-60, or over 60 years. The subject may suffer from or have been diagnosed with a condition that causes increased risk of severe illness due to a viral infection, or for which a standard of care treatment causes increased risk of severe illness due to a viral infection. Such conditions include, for example, cancer, chronic kidney disease, chronic lung disease (such as chronic obstructive pulmonary disease (COPD), asthma, interstitial lung disease, cystic fibrosis, and pulmonary hypertension), dementia or other neurological condition, diabetes type 1 or type 2, down syndrome, heart conditions (such as heart failure, coronary artery disease, cardiomyopathies, or hypertension), immunocompromised state, liver disease, overweight (for example, having a body mass index of 25 or greater), obesity (for example, having a body mass index of 30 or greater), pregnancy, sickle cell disease or thalassemia, being a current or former smoker, having received a solid organ or blood stem cell transplant, stroke or cerebrovascular disease, substance use disorders, a concurrent viral infection, such as HIV infection, SARS-COV-2 infection, influenza infection, or MERS infection, or any indication identified by the U.S. Center for Disease Control and Prevention (CDC) as increasing risk of severe illness or for which standard of care causes increased risk of severe illness due to viral infection.

In any of the methods, and any aspects of the foregoing, the compound or the extract is administered in an effective amount. Suitable effective amounts are described in more detail herein. In any of the methods, and any of the aspects of the foregoing, the compound or the extract is administered in a therapeutically effective amount. In any of the methods, and any aspects of the foregoing, the administering step may comprise administering a single dose of a compound or extract according to a course of treatment (where the dose may contain an effective amount). In any of the methods, and any aspects of the foregoing, the administering step may comprise administering more than one dose of a compound or extract according to a course of treatment (where one or more doses may contain an effective amount). The amount of a compound or extract in each dose administered during a course of treatment is not required to be the same. For example, the administering step may comprise administering at least one loading dose and at least one maintenance dose during a course of treatment. Dosing is described in more details herein.

The compound may be administered as a prophylactic treatment. The compound may be administered as a cosmetic or therapeutic treatment.

The compounds disclosed herein may be used in various applications, e.g., cosmetic and/or therapeutic applications. The compounds may be administered in an effective amount for an intended use, e.g., a cosmetic or a therapeutic application. In some embodiments, a composition may comprise a concentration or amount, e.g., an effective amount, of the compound sufficient to have a desired cosmetic effect. In some embodiments, a composition may comprise a concentration or amount, e.g., an effective amount, of the compound sufficient to have a desired therapeutic effect.

An amount and/or frequency of administration may be sufficient to induce mitochondrial biogenesis. An amount and/or a frequency of administration may be sufficient to treat, inhibit, or prevent the progression of at least one of mitochondrial dysfunction, a disease or condition associated with mitochondrial dysfunction or a symptom thereof, an aging-associated chronic condition associated with mitochondrial dysfunction or a symptom thereof, and/or a decrease in energy level or vitality. An amount and/or frequency of administration may be sufficient to modify a score of a parameter on a qualitative scale, as graded by the subject or a clinical grader. The qualitative scale may comprise the following categories: none (best possible condition), mild, moderate, severe (worst possible condition). The qualitative scale may refer to perceived or actual energy levels and/or vitality.

In some aspects, administering an effective amount of the compound may limit or inhibit at least one of mitochondrial dysfunction, a disease or condition associated with mitochondrial dysfunction or a symptom thereof, an aging-associated chronic condition associated with mitochondrial dysfunction or a symptom thereof, and/or a decrease in energy level or vitality. For example, the effective amount of the compound may slow progression of the at least one of mitochondrial dysfunction, a disease or condition associated with mitochondrial dysfunction or a symptom thereof, an aging-associated chronic condition associated with mitochondrial dysfunction or a symptom thereof, and/or a decrease in energy level or vitality. In some embodiments, administering an effective amount of the compound may promote mitochondrial biogenesis, mitochondrial function, and/or an increase in energy level or vitality.

In some aspects, administering an effective amount of the compound may increase expression of at least one protein selected from PGC-1a, TFAM, NRF-1, and COX II. Administering an effective amount may decrease expression or inhibit an increase of expression of at least one protein selected from FGF-21 and IL-6, or any cytokine related to viral infection. Administering an effective amount may increase or inhibit a decrease in at least one of ATP-linked respiration, maximal respiration and reserve capacity in subjects infected with SARS-CoV-2. Administering an effective amount may modulate, e.g., increases or decreases, viral protein interaction with one or more host mitochondrial genes, e.g., MRPS2, MRPS5, MRPS25, MRPS27, NDUFAF1, NDUFB9, NDUFAF2, ATPIB1, ATP6VIA, ACADM, AASS, PMPCB, PITRM1, COQ8B, PMPCA, and Tomm70. Administering an effective amount may increase expression of ACE2.

The amount of the compound or composition may be effective in treating or preventing an ovarian disease or condition in the subject. The amount of the composition may be effective in improving or enhancing fertility and/or extending reproductive longevity. In certain embodiments, administering an effective amount, e.g., administering a therapeutically or cosmetically effective amount, may induce mitochondrial biogenesis and/or improve mitochondrial function. For instance, in some embodiments, administering an effective amount may increase oocyte mitochondrial mass or prevent a decrease in oocyte mitochondrial mass.

In some aspects, the compound may be administered prior to onset of the disease or condition in the subject. The compound may be administered during incidence of the disease or condition in the subject. The compound may be administered subsequent to at least partial reduction of the disease or condition in the subject.

The compound may be administered in response to a trigger or warning sign of an ovarian disease or condition, e.g., prolonged or irregular menstrual cycles, decreased ovulation events, and/or menstrual cramps or pain.

The compound may be administered in response to a trigger or warning sign of a mitochondrial dysfunction or aging-associated condition, e.g., aging, premature aging, habitual sleep conditions, weight loss, ultraviolet (UV) light exposure, treatment with a chemical or therapeutic agent, e.g., chemotherapy and/or radiation therapy, smoking, dehydration, or immersion. The subject may be predisposed for a mitochondrial dysfunction condition, e.g., based on age, race, skin type, eye color, habit, or heredity.

A method may further comprise determining whether the subject is in need of treatment or administration.

The composition comprising the compound may additionally comprise a moisturizing agent, deodorizing agent, scent, colorant, insect repellant, cleansing agent, or UV-blocking agent. The composition may include microspheres or microcapsules.

The composition may be formulated for immediate release or extended release. The composition may be formulated for controlled or sustained release. For example, the composition may be formulated for sustained release over a period of 6 hours, 12 hours, 18 hours, 24 hours, 36 hours, 48 hours, or more.

The subject may be characterized as having normal mitochondrial function. The subject may be characterized as having reduced mitochondrial function. The subject may be characterized as having an increased level of circulating mtDNA, e.g., plasma mtDNA and/or cytoplasmic mtDNA.

The subject may be characterized by age-related reduced fertility, infertility, and/or ovarian aging. For instance, in some embodiments, the subject or recipient subject is 40 years or older.

The subject may be characterized as experiencing premature aging or a symptom of premature aging. For example, the subject may be characterized as experience premature reproductive aging, which may include one or more of primary ovarian insufficiency (POI), early onset prolonged or irregular menstrual cycles, early onset decreased ovulation events, premature menopause or perimenopause, and/or ovarian inflammation associated with premature reproductive aging. In such embodiments, the subject may be younger than 40 years.

In some embodiments, the subject may have a diminished ovarian reserve (DOR). In other embodiments, the subject may have a normal ovarian reserve (NOR).

The subject may be pre-menopausal, perimenopausal, or post-menopausal.

The subject may suffer from reduced production of reproductive hormones, e.g., estrogen and/or progesterone, increased production of follicle-stimulating hormone (FSH), prolonged or irregular menstrual cycles, uterine or vaginal atrophy, e.g., decreased endometrial glands, diffuse glandular breakdown (apoptotic bodies) of the uterus, acute inflammation of the uterus, and/or decreased vaginal epithelial thickness, decreased ovulation events, ovarian inflammation, and/or infertility.

The subject may be characterized by dysfunctional folliculogenesis, e.g., follicle depletion or follicle dysfunction. For example, the subject may have decreased tertiary follicle counts, e.g., early antral, antral, and/or pre-ovulatory, and/or decreased corpora lutea (CL) follicle counts.

The subject may be characterized by down-regulation of estrogen receptors (ER) α and β in the ovary, reduced ESR1 or ESR2 gene expression, increased miR-206-3p RNA expression, reduced ovarian production of 17β-estradiol (E2), and/or increased lipid-laden ovarian stromal cells.

The subject may be characterized by a reduced anti-Mullerian hormone (AMH) level, e.g., less than about 80 ng/ml, an increased follicle stimulating hormone (FHL) level, e.g., greater than about 10 IU/L, an increased estradiol level, and/or a reduced antral follicle count (AFC), e.g., less than about 5-7 total follicles.

In certain embodiments, the subject may be characterized as suffering from one or more of obesity, diabetes, chronic inflammation, high blood sugar, an autoimmune disease, and/or poor lifestyle factors, such as smoking, alcohol use, drug use, exposure to chemicals (e.g., bisphenol A, advanced glycation end products (AGE)) and/or radiation, poor nutrition, e.g., increased ingestion of charred foods, increased ingestion of saturated fats, and/or reduced ingestion of fruits and vegetables, and/or reduced exercise.

In certain embodiments, the subject may be undergoing in vitro fertilization (IVF). In certain embodiments, the subject may have previously attempted IVF, successfully or unsuccessfully. The subject may be a poor responder to IVF. In other embodiments, the subject may be a normal responder to IVF.

The subject may have a mitochondrial DNA mutation in an oocyte to be fertilized to form the embryo or the recipient oocyte. The mutation may be somatic or inherited. In certain embodiments, the mutation may be, e.g., a T414G transverse mutation.

The subject may be characterized by decreased levels of mitochondrial DNA (mtDNA) in an oocyte to be fertilized to form the embryo. The subject may be characterized by reduced oocyte quality, e.g., chromosomal misalignment and/or spindle malformation.

The subject may be characterized by a mitofusin (Mfn1) gene abnormality, a dynamic related protein 1 (Drp1) gene abnormality, a caseinolytic peptidase P (Clpp) gene abnormality, a growth differentiation factor 9 (GDF9) gene abnormality, a fragile-X mental retardation 1 (FMR1) gene abnormality, a transcription factor A (TFAM) gene abnormality, a mitochondrial DNA polymerase gamma (PolgA) gene abnormality, a mitochondrial inner membrane peptidase (IMP) gene abnormality, and/or a mitochondrial ABC transporter (MDR-1) gene abnormality. The subject may be characterized by decreased gene expression of PPARy, PGC-1a, PGC-1b, ERR, NRF-1, NRF-2, SIRT1, SIRT3, SIRT4, and/or SIRT5. The subject may be characterized by decreased AMP-activated protein kinase (AMPK) and or PTEN-induced kinase 1 (PINK1) protein expression and/or increased mammalian target of rapamycin (mTOR) protein expression.

The subject may be characterized by decreased gene expression of superoxide dismutase (SOD) and/or increased levels of Aldh3A2 enzyme. The subject may be characterized by decreased mRNA expression of Prdx3, Prdx4, and/or Txn2 enzyme.

The subject may be characterized by a neurologic condition. In some embodiments, the subject may suffer from Turner's Syndrome, galactosemia, and/or Fragile X Syndrome. In some embodiments, the subject may be characterized by progressive external ophthalmoplegia (POE), spinocerebellar ataxia with epilepsy (SCAE), and/or mitochondrial encephalomyopathy, lactic acidosis, and stroke-like episodes (MELAS).

In certain embodiments, the subject may be characterized by one or more of: an inflammatory phenotype in the skin, a change in mitochondrial protein expression, reduced expression of mitochondrial oxidative phosphorylation complexes, reduced stability of mitochondrial oxidative phosphorylation complexes, a decrease in collagen content of the skin, increased epidermal thickness, increased epidermal hyperplasia, acanthosis, hyperkeratosis, increased expression of at least one gene selected from the group consisting of: NF-κB, COX-2, INF-1, CCL5, MMP1, MMP2, MMP9, MMP13, IGF1R, VEGF, and MRPS5, decreased expression of at least one of gene selected from the ground consisting of TIMP1, KLOTHO, COL1A1, MTCO2, TFAM, and VDAC, and increased inflammatory infiltrate in skin. The subject may be characterized as having decreased expression of at least one protein selected from PGC-1a, TFAM, NRF-1, and COXII.

In some aspects, a method may further comprise administering a second amount of the compound to the subject. The second amount may be administered as a second dose of the same formulation. The second amount may be administered in another formulation. The second amount may be administered in another formulation by the same route of administration, e.g., a topical solution or oil with a shampoo, conditioner, spray, cream, gel, body wash, soap, or lotion. The second amount may be administered by another route of administration, e.g., each amount may independently be administered topically, parenterally, or enterally.

In some aspects, a method may further comprise administering a second compound to the subject. The second compound may be a compound that is a constituent of the same extract. The second compound may be a compound that is a constituent of another extract. The second compound may be a compound having a similarity score of at least 95% with the first compound. The second compound may be administered in the same formulation. The second compound may be administered in another formulation.

The compound may be administered as part of a combination therapy. The method may further comprise administering a second treatment in combination with the compound. The compound may be administered for a period of time prior to initiating the second treatment. The compound may be administered concurrently with the second treatment. The compound may be administered for a period of time subsequent to ceasing the second treatment. The second treatment may be administered via an alternate mode of administration. The second treatment may be a cosmetic and/or therapeutic treatment.

The compound may be administered in combination with a second agent, e.g., cosmetic or therapeutic agent, approved to treat or commonly used to treat the disease or condition or a symptom thereof.

The compound may be administered in combination with a standard of care treatment for improving fertility. For instance, the compound may be administered in combination with one or more of clomiphene, tamoxifen, letrozole, metformin, gonadotrophins, gonadotrophin-releasing hormone, dopamine agonists, and other hormonal treatments.

The compound may be administered in combination with a standard of care treatment for treating an ovarian disease or condition. For instance, the compound may be administered in combination with one or more of hormone therapy, e.g., estrogen, progesterone, testosterone, or a synthetic form thereof, selective serotonin reuptake inhibitors (SSRIs), gabapentin, clonidine, hormonal contraceptives, gonadotropin-releasing hormone (GnRH) agonists or antagonists, heat therapy, pain relievers, nonsteroidal anti-inflammatory drugs (NSAID), such as ibuprofen or other analgesics, spironolactone, eflornithine, electrolysis, chemotherapy, radiation therapy, surgery, e.g., hysterectomy, bilateral salpingo-oophorectomy, and debulking surgery, and lifestyle changes, such as weight loss, improved nutrition, and increased physical activity.

The compound may be administered in combination with caffeine, B vitamins (e.g., B1, B2, B3, B5, B6, B8, B9 and/or B12), vitamin C, vitamin D, iron, magnesium, and/or zinc.

The compound may be administered in combination with UV-blocking agent, moisturizer, sunscreen, wrinkle cream, retinoid, alpha-hydroxy acid, beta-hydroxy acid, squalene, antioxidant, tretinoin, glycosaminoglycan (GAG), lactic acid, malic acid, citric acid, tartaric acid, hydroquinone, kojic acid, L-ascorbic acid, licorice extract. N-acetylglucosamine, niacinamide, soy, dermal filler or injection, e.g. hyaluronic acid or calcium hydroxylapatite, botulinum toxin, laser resurfacing procedure, ultrasound therapy, chemical peel, e.g. glycolic acid peel, trichloroacetic acid or salicylic acid, or dermabrasion procedure.

The compound may be administered in combination with a surgical procedure, e.g., fallopian tube surgery, laparoscopic surgery to remove or destroy cysts or submucosal fibroids, or laparoscopic ovarian drilling.

The compound may be administered in combination with an antioxidant. Exemplary antioxidants include CoQ10, vitamin C, vitamin E, carotenoids, e.g., beta-carotene, minerals, e.g., selenium and manganese, glutathione, lipoic acid, flavonoids, betaflavinoids, phenols, polyphenols, phytoestrogens, mitoquinol mesylate, and ubiquinone.

The Fucus compound may be administered in combination with an Emblica extract or a compound constituent of an Emblica extract. The Fucus compound may be administered in combination with a chebula extract or a compound constituent of a chebula extract. The Emblica compound may be administered with a Fucus extract or a compound constituent of a Fucus extract. The Emblica compound may be administered with a chebula extract or a compound constituent of a chebula extract. In accordance with certain embodiments, the combination of two or more of an Emblica compound, a Fucus compound, and a chebula compound may provide synergistic effects in the treatment of the disease or condition.

In accordance with one or more embodiments, an effective amount of the compound may be administered to a face of a subject. In accordance with one or more embodiments, the compound may be administered to the scalp of the subject. In accordance with one or more embodiments, the compound may be administered to the body of the subject. For example, the compound may be applied to one or more of the more of the forehead, eye region, neck, scalp, head, shoulder, arm, hands, leg, underarm, torso, chest, feet, knee, ankle, back, buttock, or genitals of the subject.

In accordance with one or more embodiments, an effective amount of the compound may be administered enterally or parenterally.

### In Vitro Treatment Methods, Such as In Vitro Fertilization (IVF)

In accordance with certain embodiments, the subject may be undergoing in vitro fertilization (IVF). The subject may have previously attempted IVF, successfully or unsuccessfully. The subject may be a poor responder to IVF. In other embodiments, the subject may be a normal responder to IVF.

During IVF, oocytes are typically retrieved from the ovary of a subject and placed in vitro with active, motile sperm to produce one or more embryo. Viable embryos may be selected for transfer into the uterus of the subject or a surrogate. Additional viable embryos may be preserved for future transfer. The methods disclosed herein may involve improving embryo development or improving embryo fertilization rate.

Thus, in some embodiments, the embryo may be produced by IVF. The method may comprise measuring mitochondrial content of the embryo and selecting the embryo responsive to the measurement of mitochondrial content being within a predetermined range. The predetermined range may correspond to a healthy range, for example, a range associated with a normal ovarian reserve (NOR) and fertility.

The methods may comprise administering the compositions disclosed herein to the subject or to the embryo. For instance, the method may comprise treating the embryo by introducing the composition to a culture media of the embryo during development. The composition may be introduced in an amount effective to improve embryo development or fertilization rate. The method may comprise introducing antioxidants to the culture media in combination with the composition.

In certain embodiments, the methods may include transferring ooplasm from a donor oocyte to a recipient oocyte of the subject to be fertilized to form the embryo. In general, the donor oocyte may have a mitochondrial DNA (mtDNA) content greater than the recipient oocyte of the subject. The donor oocyte may be autologous. The donor oocyte may be allogeneic. The donor oocyte may be xenogeneic. In such embodiments, the composition may have been administered to a donor subject, the recipient subject, and/or the embryo.

The subject may have a mitochondrial DNA mutation in the oocyte to be fertilized to form the embryo. The mutation may be somatic or inherited. In certain embodiments, the mutation may be, e.g., a T414G transverse mutation. In some embodiments, the subject may be characterized by decreased levels of mitochondrial DNA (mtDNA) in the oocyte to be fertilized to form the embryo. The subject may be characterized by reduced oocyte quality, e.g., chromosomal misalignment and/or spindle malformation.

In some embodiments, the ooplasm transfer may be a complete transfer. In other embodiments, the ooplasm transfer may be partial. The partial ooplasm transfer may be by electrofusion or direct ooplasmic injection.

The ooplasm transfer may be performed by one of several methods. In some embodiments, the ooplasm transfer may be performed by modified intracytoplasmic sperm injection (ICSI). In some embodiments, the ooplasm transfer may be performed by autonomous germline mitochondrial energy transfer (AUGMENT) on the oocyte. The ooplasm transfer may be performed by nuclear genome transfer, e.g., oocyte spindle transfer, germinal vesicle (GV) transfer, pronuclear transfer (PNT), or polar body nuclear transfer (PBNT).

In some embodiments, the method may comprise using CRISPR/Cas 9 gene editing technology on mitochondrial DNA (mtDNA) of an oocyte of the subject to be fertilized to form the embryo. In embodiments which include ooplasm transfer, the methods may comprise using CRISPR/Cas 9 gene editing technology on the mtDNA of the donor oocyte or the recipient oocyte.

In some embodiments, the method may comprise introducing stem cell generated mitochondrial DNA (mtDNA) into an oocyte of the subject to be fertilized to form the embryo. Thus, in certain embodiments, mtDNA transfer may occur without a donor oocyte.

Thus, in accordance with another aspect, there is provided a preparation comprising mtDNA as a therapeutic agent. The preparation may comprise an effective amount of donor mtDNA in a therapeutically acceptable carrier. The mtDNA may be edited by CRISPR/Cas 9. The mtDNA may be stem cell generated. The mtDNA may be in the form of a complete ooplasm transfer or a partial ooplasm transfer.

Kits comprising the preparation comprising mtDNA are also disclosed. The kits may additionally comprise a compound of the disclosure, or a pharmaceutically acceptable form thereof.

### Dosage and Administration

In accordance with the methods, the compounds of the disclosure are administered to the subject (or are contacted with cells of the subject) in an effective amount.

In certain embodiments, therapeutically the effective amount of a compound of the disclosure ranges from about 0.05 mg/kg/day to about 50 mg/kg/day. In certain embodiments, the effective amount ranges from about 0.05 mg/kg/day to about 40 mg/kg/day. In certain embodiments, the effective amount ranges from about 0.05 mg/kg/day to about 30 mg/kg/day. In certain embodiments, the effective amount ranges from about 0.05 mg/kg/day to about 20 mg/kg/day. In certain embodiments, the effective amount ranges from about 0.05 mg/kg/day to about 10 mg/kg/day. In certain embodiments, the effective amount ranges from about 0.05 mg/kg/day to about 8 mg/kg/day. In certain embodiments, the effective amount ranges from about 0.05 mg/kg/day to about 6 mg/kg/day. In certain embodiments, the effective amount ranges from about 0.05 mg/kg/day to about 4 mg/kg/day. In certain embodiments, the effective amount ranges from about 0.05 mg/kg/day to about 3 mg/kg/day. In certain embodiments, the effective amount ranges from about 0.05 mg/kg/day to about 2 mg/kg/day. In certain embodiments, the effective amount ranges from about 0.05 mg/kg/day to about 1 mg/kg/day. In certain embodiments, the effective amount ranges from about 0.05 mg/kg/day to about 0.8 mg/kg/day. In certain embodiments, the effective amount ranges from about 0.05 mg/kg/day to about 0.6 mg/kg/day. In certain embodiments, the effective amount ranges from about 0.05 mg/kg/day to about 0.4 mg/kg/day. In certain embodiment, the amounts per day described above are administered according to a course of treatment and may be administered in a single dose or in more than 1 dose per day. The amounts per day described above may be administered according to a course of treatment and administered in one dose (q.d.) or two doses each day (b.i.d.), wherein the amount of the compound of the disclosure in each dose need not be the same.

In certain embodiments, each dose is administered according to a course of treatment. As used herein, the term “dose” refers to an amount of a compound of the disclosure administered at a given time point according to a course of treatment. For example, if a course of treatment for a compound of the disclosure is b.i.d (2 times/administrations per day) for 7 days, the two administrations on each of days 1-7 would each comprise administering a dose of a compound of the disclosure (for 2 doses each day). In certain embodiments, a dose is administered q.d. (1 time/administration per day) according to a course of treatment. In certain embodiments, a dose is administered b.i.d. according to a course of treatment. In certain embodiments, a dose is administered t.i.d. (three times/administrations per day) according to a course of treatment.

When 2 or more doses are administered on a given day according to a course of treatment, each dose administered according to the course of treatment may contain the same amount of a compound of the disclosure or one or more of doses administered according to the course of treatment may contain a greater or lesser amount of a compound of the disclosure as compared to another dose administered according to the course of treatment. For example, if a course of treatment for a compound of the disclosure is b.i.d for 7 days, the first dose administered on day 1 may contain a first amount (i.e., 2 mg/kg) and the second dose administered on day 1 may contain a second amount (i.e., 0.5 mg/kg). As another example, if a course of treatment for a compound of the disclosure is b.i.d for 7 days, the first dose administered on day 1 may contain a first amount (i.e., 2 mg/kg), the second dose administered on day 1 may contain a second amount (i.e., 0.5 mg/kg), the two doses administered on each of days 2-4 may contain the second amount, and the two doses administered on each of days 5-7 may contain a third amount (i.e., 1 mg/kg).

A dose may be further divided into a sub-dose. Any given dose may be delivered in a single unit dose form or more than one unit dose form. For example, a dose when given by IV administration may be provided as a single IV infusion (i.e., a single 5 mg/kg IV infusion) or as two or more IV infusions administered one after the other (i.e., two 2.5 mg/kg IV infusions). Further, a sub-dose might be, for example, a number of discrete loosely spaced administrations, such as multiple inhalations from an insufflator, by application of a plurality of drops into the eye, or multiple tablets for oral administration.

In certain embodiments, more than one dose of a compound of the disclosure is administered during a course of treatment. Therefore, in the methods described herein, the methods may comprise the administration of multiple doses during the course of treatment. In certain embodiments, the course of treatment may range from months to years. In certain embodiments, the course of treatment may range from 2 days to 1 month, from 2 days to 3 weeks, from 2 days to 2 weeks, or from 2 days to 1 week. In certain embodiments, the course of treatment may range from 1 year to 20 years or longer. In certain embodiments, a dose is delivered at least 1 time per day (i.e., 1 to 3 times) during the course of treatment. In certain embodiments, a dose is not administered every day during the course of treatment (for example, a dose is be administered at least 1 timer per day every other day, every third day, every week, or every month during the course of treatment). Furthermore, the amount of a compound of the disclosure in each dose need not be the same as discussed above. In certain embodiments, of the foregoing, one or more doses, or all of the doses, contain an effective amount of a compound of the disclosure.

In one embodiment, a course of treatment may comprise administering at least one dose as a loading dose and at least one dose as a maintenance dose, wherein the loading dose contains a greater amount of a compound of the disclosure as compared to the maintenance dose (such as, but not limited to, 2 to 10 times higher). In one aspect of this embodiment, the loading dose is administered initially, either as a single administration or more than one administration, followed by administration of one or more maintenance doses through the remaining course of treatment. For example, for a course of treatment that is q.d. every week for 10 years, a loading dose of 3 mg/kg may be administered as the first dose on week 1 of the course of treatment, followed by maintenance doses of 0.5 mg/kg every week for the remainder of the course of treatment. Furthermore, a loading dose may be given as a dose that is not the first dose administered during a course of treatment. For example, a loading dose may be administered as the first dose on week and as a dose on one or more additional weeks (for example, weeks 10 and 20).

In one embodiment, a course of treatment may comprise administering a first dose formulated in a first composition and administering at least one second or subsequent dose formulated in a second composition. The first composition and the second composition may be the same formulation. The first composition and the second composition may be different formulations.

Pharmaceutical, Cosmetic, and/or Dietary Compositions

Pharmaceutical compositions are provided that comprise an amount, e.g., an effective amount, of a compound of the disclosure. In one embodiment, such pharmaceutical compositions contain a therapeutically effective amount of a compound of the disclosure. In addition, other active agents may be included in such pharmaceutical compositions. Additional active agents to be included may be selected based on the disease or condition to be treated.

The pharmaceutical compositions disclosed may comprise one or more compound of the disclosure, alone or in combination with additional active agents, in combination with a pharmaceutically acceptable carrier and/or excipient and/or in combination with a cosmetically acceptable carrier and/or excipient. Such pharmaceutical compositions may be used in the manufacture of a medicament for use in the methods described herein. The compounds of the disclosure are useful in both free form and in the pharmaceutically acceptable forms, such as pharmaceutically acceptable salts.

The pharmaceutically acceptable carriers and/or excipients and/or cosmetically acceptable carriers and/or excipients are well-known to those who are skilled in the art. The choice of carrier and/or excipient will be determined in part by the particular compound(s), as well as by the particular method used to administer the compound composition. Accordingly, there is a wide variety of suitable formulations of the composition of the disclosure. The following methods and excipients are merely exemplary and are in no way limiting. Suitable carriers and excipients include solvents such as water, alcohol, and propylene glycol, solid absorbants and diluents, surface active agents, suspending agent, tableting binders, lubricants, flavors, and coloring agents. The pharmaceutically and/or cosmetically acceptable carriers can include polymers and polymer matrices. Examples of acceptable carriers include carboxymethyl cellulose, crystalline cellulose, glycerin, gum arabic, lactose, magnesium stearate, methyl cellulose, powders, saline, sodium alginate, sucrose, starch, talc and water, among others. Typically, the acceptable carrier is chemically inert to the active agents in the composition and has no detrimental side effects or toxicity under the conditions of use.

Surfactants such as, for example, detergents, are also suitable for use in the formulations. Specific examples of surfactants include polyvinylpyrrolidone, polyvinyl alcohols, copolymers of vinyl acetate and of vinylpyrrolidone, polyethylene glycols, benzyl alcohol, mannitol, glycerol, sorbitol or polyoxyethylenated esters of sorbitan; lecithin or sodium carboxymethylcellulose; or acrylic derivatives, such as methacrylates and others, anionic surfactants, such as alkaline stearates, in particular sodium, potassium or ammonium stearate; calcium stearate or triethanolamine stearate; alkyl sulfates, in particular sodium lauryl sufate and sodium cetyl sulfate; sodium dodecylbenzenesulphonate or sodium dioctyl sulphosuccinate; or fatty acids, in particular those derived from coconut oil, cationic surfactants, such as water-soluble quaternary ammonium salts of formula N+R′R″R′″R″″Y−, in which the R radicals are identical or different optionally hydroxylated hydrocarbon radicals and Y− is an anion of a strong acid, such as halide, sulfate and sulfonate anions; cetyltrimethylammonium bromide is one of the cationic surfactants which can be used, amine salts of formula N+R′R″R′″, in which the R radicals are identical or different optionally hydroxylated hydrocarbon radicals; octadecylamine hydrochloride is one of the cationic surfactants which can be used, non-ionic surfactants, such as optionally polyoxyethylenated esters of sorbitan, in particular Polysorbate 80, or polyoxyethylenated alkyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide, amphoteric surfactants, such as substituted lauryl compounds of betaine.

Exemplary pharmaceutically acceptable carriers and/or excipients and/or cosmetically acceptable carriers and/or excipients include water, silica, glycerin, dimethicone, butylene glycol, pentylene glycol, ethoxydiglycol, polyacrylate-13, pentapeptide-34 trifluoroacetate, polyisobutene, lysolecithin, sclerotium gum, pullulan, polysorbate 20, diethylhexyl syringylidenemalonate, caprylyl glycol, glyceryl stearate, PEG-100 stearate, cetearyl alcohol, butyrospermum parkii (shea) butter, acetyl tetrapeptide-2, betaine, melanin, tocopheryl acetate, tocopherol, hydroxyacetophenone, caprylic/capric triglyceride, batyl alcohol, C12-15 alkyl benzoate, panthenol, ceteareth-20, xanthan gum, ethylhexylglycerin, disodium EDTA, propanediol, caprylyl glycol, potassium sorbate, sorbic acid, and phenoxyethanol.

The compounds of the disclosure and pharmaceutical compositions containing such compounds as described in the instant disclosure can be administered by any conventional method available for use in conjunction with pharmaceuticals, either as individual therapeutic agents or in combination with additional therapeutic agents.

In one embodiment, the compounds of the disclosure are administered in an effective amount, whether alone or as a part of a pharmaceutical composition. The effective amount and the dosage administered will, of course, vary depending upon known factors, such as the pharmacodynamic characteristics of the particular agent and its mode and route of administration, the age, health and weight of the recipient; the severity and stage of the disease state or condition; the kind of concurrent treatment; the frequency of treatment; and the effect desired.

The total amount of the compound administered will also be determined by the route, timing and frequency of administration as well as the existence, nature, and extent of any adverse side effects that might accompany the administration of the compound and the desired physiological effect. It will be appreciated by one skilled in the art that various conditions or disease states, in particular chronic conditions or disease states, may require prolonged treatment involving multiple administrations.

In these pharmaceutical or cosmetic compositions, the compound(s) of the disclosure will ordinarily be present in an amount of about 0.5-95% weight based on the total weight of the composition, or about 0.1-99.9% weight based on the total weight of the composition. Multiple dosage forms may be administered as part of a single treatment.

The active agent can be administered enterally in solid dosage forms, such as capsules, tablets, and powders, or in liquid dosage forms, such as milk, elixirs, syrups and suspensions. It can also be administered parenterally, in sterile liquid dosage forms. The compound(s) of the disclosure can also be administered intranasally (nose drops) or by inhalation via the pulmonary system, such as by propellant based metered dose inhalers or dry powders inhalation devices. Other dosage forms include topical administration, such as administration transdermally, via patch mechanism or ointment.

Formulations suitable for enteral or oral administration may be liquid solutions, such as an effective amount of the compound(s) dissolved in diluents, such as milk, water, saline, buffered solutions, infant formula, other suitable carriers, or combinations thereof. Formulations suitable for enteral or oral administration of the compounds of the disclosure are known in the art as exemplified by: Shaji, et al., Indian J Pharm Sci. 2008 May-June; 70 (3): 269-277; Bruno, et al., Ther Deliv. 2013 November; 4 (11): 1443-1467; Ibrahim, et al., DARU Journal of Pharmaceutical Sciences, 2020, 28, 403-416. The compound(s) can then be mixed to the diluent just prior to administration. In an alternate embodiment, formulations suitable for enteral or oral administration may be capsules, sachets, tablets, lozenges, and troches. In each embodiment, the formulation may contain a predetermined amount of the compound(s) of the disclosure, as solids or granules, powders, suspensions and suitable emulsions. Liquid formulations may include diluents, such as water and alcohols, for example, ethanol, benzyl alcohol, propylene glycol, glycerin, and the polyethylene alcohols, either with or without the addition of an acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers.

Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such carriers as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the patient, and aqueous and non-aqueous sterile suspensions that can include suspending agents, solubilizers, thickening agents, stabilizers, and preservatives. The compound(s) can be administered in a physiologically acceptable diluent in an acceptable carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of an acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldialkylammonium halides, and alkylpyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkyl.beta.-aminopropionates, and 2-alkylimidazoline quaternary ammonium salts, and (e) mixtures thereof.

The parenteral formulations typically contain from about 0.5% to about 50% by weight of the compound(s) in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

The compound(s) of the disclosure can be formulated into aerosol formulations to be administered via nasal or pulmonary inhalation. These aerosol formulations can be placed into pressurized acceptable propellants, such as dichlorodifluoromethane, propane, and nitrogen. Such aerosol formulations may be administered by metered dose inhalers. They also may be formulated as pharmaceuticals for non-pressured preparations, such as in a nebulizer or an atomizer.

The compound(s) of the disclosure, alone or in combination with other suitable components, may be administered in an aqueous solution as a nasal or pulmonary spray and may be dispensed in spray form by a variety of methods known to those skilled in the art. Systems for dispensing liquids as a nasal spray are disclosed in U.S. Pat. No. 4,511,069. The formulations may be presented in multi-dose containers, for example in the sealed dispensing system disclosed in U.S. Pat. No. 4,511,069. Additional aerosol delivery forms may include, e.g., compressed air-, jet-, ultrasonic-, and piezoelectric nebulizers, which deliver the active agent dissolved or suspended in a pharmaceutical solvent, e.g., water, ethanol, or a mixture thereof.

Nasal and pulmonary solutions of the disclosure may typically comprise the drug or drug to be delivered, optionally formulated with a surface-active agent, such as a nonionic surfactant (e.g., polysorbate-80), and one or more buffers. In some embodiments, the nasal spray solution further comprises a propellant. The pH of the nasal spray solution is optionally between about pH 3.0 and 6.0, or 4.5+/−0.5. Suitable buffers for use within these compositions are as described above or as otherwise known in the art. Other components may be added to enhance or maintain chemical stability, including preservatives, surfactants, dispersants, or gases. Suitable preservatives include, but are not limited to, phenol, methyl paraben, paraben, m-cresol, thiomersal, chlorobutanol, benzylalkonimum chloride, and the like. Suitable surfactants include, but are not limited to, oleic acid, sorbitan trioleate, polysorbates, lecithin, phosphatidyl cholines, and various long chain diglycerides and phospholipids. Suitable dispersants include, but are not limited to, ethylenediaminetetraacetic acid, and the like. Suitable gases include, but are not limited to, nitrogen, helium, chlorofluorocarbons (CFCs), hydrofluorocarbons (HFCs), carbon dioxide, air, and the like.

Within alternate embodiments, nasal and pulmonary formulations are administered as dry powder formulations comprising the active agent in a dry, usually lyophilized, form of an appropriate particle size, or within an appropriate particle size range, for intranasal delivery. Minimum particle size appropriate for deposition within the nasal or pulmonary passages is often about 0.5 μm. mass median equivalent aerodynamic diameter (MMEAD), commonly about 1 μm MMEAD, and more typically about 2 μm MMEAD. Maximum particle size appropriate for deposition within the nasal passages is often about 10 μm MMEAD, commonly about 8 μm MMEAD, and more typically about 4 μm MMEAD. Intranasally and pulmonaryly respirable powders within these size ranges can be produced by a variety of conventional techniques, such as jet milling, spray drying, solvent precipitation, supercritical fluid condensation, and the like. These dry powders of appropriate MMEAD can be administered to a patient via a conventional dry powder inhaler (DPI), which relies on the patient's breath, upon pulmonary or nasal inhalation, to disperse the power into an acrosolized amount. Alternatively, the dry powder may be administered via air-assisted devices that use an external power source to disperse the powder into an aerosolized amount, e.g., a piston pump.

To formulate compositions for nasal or pulmonary delivery, the active agent can be combined with various pharmaceutically and/or cosmetically acceptable additives, as well as a base or carrier for dispersion of the active agent(s). Desired additives include, but are not limited to, pH control agents, such as arginine, sodium hydroxide, glycine, hydrochloric acid, citric acid, etc. In addition, local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione) can be included. When the composition for nasal or pulmonary delivery is a liquid, the tonicity of the formulation, as measured with reference to the tonicity of 0.9% (w/v) physiological saline solution taken as unity, is typically adjusted to a value at which no substantial, irreversible tissue damage will be induced in the nasal mucosa at the site of administration. Generally, the tonicity of the solution is adjusted to a value of about 1/3 to 3, more typically 1/2 to 2, and most often 3/4 to 1.7.

The compound(s) of the disclosure may be dispersed in a base or vehicle, which may comprise a hydrophilic compound having a capacity to disperse the active agent and any desired additives. The base may be selected from a wide range of suitable carriers, including but not limited to, copolymers of polycarboxylic acids or salts thereof, carboxylic anhydrides (e.g., maleic anhydride) with other monomers (e.g., methyl (meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. Often, a biodegradable polymer is selected as a base or carrier, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters, etc. can be employed as carriers. Hydrophilic polymers and other carriers can be used alone or in combination, and enhanced structural integrity can be imparted to the carrier by partial crystallization, ionic bonding, crosslinking and the like. The carrier can be provided in a variety of forms, including, fluid or viscous solutions, gels, pastes, powders, microspheres and films for direct application to the nasal mucosa. The use of a selected carrier in this context may result in promotion of absorption of the active agent.

The compounds of the disclosure may be formulated as described in International Patent Application No. PCT/US2022/020983, filed Mar. 18, 2022, titled “COMPOSITIONS AND METHODS FOR IMPROVING MITOCHONDRIAL FUNCTION,” the entire disclosure of which is herein incorporated by reference in its entirety for all purposes.

The compounds of the disclosure may be formulated in a nanoparticle-based delivery carrier. The nanoparticle delivery systems disclosed herein may offer benefits in mitochondria-targeted delivery and improving the therapeutic ability of the compounds. Nanoparticle formulations have been shown to effectively transport drug molecules in their original form, solubilize hydrophobic drug molecules, enhance half-life of molecules, and decrease side effects and immunogenicity attributable to the molecules. The nanoparticle delivery carriers disclosed herein may be selected or designed to provide enhanced skin penetration, higher stability, site specific targeting, e.g., efficiently deliver cargo inside the mitochondrial matrix, high entrapment efficiency, and/or time-controlled, e.g., delayed or sustained, release of the compounds. Properties of the nanoparticle delivery carrier that may be designed to effectively deliver compounds of the formulation to a target site of a subject include surface chemistry, coating, structure, size, ability to aggregate, and solubility. Exemplary nanomaterials are described in “Nanotherapeutic Approaches to Target Mitochondria in Cancer,” (Mani, 2021) and “Role of Nanotechnology in Cosmeceuticals: A Review of Recent Advances,” (Kaul, 2018), each of which is incorporated herein by reference in its entirety for all purposes.

The nanoparticle delivery carrier may comprise and/or be functionalized with carbon-based nanomaterials, liposomal delivery vehicles, polymeric nanocarriers, micelles, dendrimers, lipophilic cations, solid-lipid nanoparticles (SLN), peptide-based nanomaterials, nanostructured lipid carriers (NLC), niosomes, nanoemulsions, metal nanoparticles, nanospheres, polymerosomes, cubosomes, and combinations thereof. The delivery carrier may be designed to for mitochondrial targeting or specific cell type targeting. Exemplary mitochondrial targeting agents include antibodies, polymeric functional moieties, such as PEG, lipophilic cations, such as triphenylphosphine (TPP), and peptides, such as mitochondrial penetrating peptides (MPP). The delivery carrier may be formed of a targeting moiety, for example, encapsulating or conjugated with the compounds disclosed herein, and/or the delivery carrier may be functionalized with a surface targeting moiety. The nanoparticle carrier may be dimensioned to have an average size of about 10 to 5000 nm, for example 10 to 50 nm, 10 to 100 nm, 50 to 500 nm, 50 to 100 nm, 100 to 500 nm, 500 to 1000 nm, or 1000 nm to 5000 nm, which can be selected based on the target tissue, compound to be delivered, and other properties of the nanomaterial.

Exemplary carbon-based nanomaterials include carbon dots (C-dots or CD), carbon nanotubes (CNT), graphene derivatives, nanodiamonds (ND), quantum dots (QD), and magnetic nanoparticles (MNP). Carbon nanoparticles may be formed into different shapes including, for example, spherical, elliptical, tube, horn-shaped, and combinations thereof.

CNTs are graphene sheets formed into cylindrical tubes. CNTs have demonstrated low toxicity profile, good biocompatibility, and targeted accumulation. CNTs of the disclosure may be single-walled carbon nanotubes (SWCNT) and/or multi-walled carbon nanotubes (MWCNT). SWCNTs have a smaller diameter, in the range of 1 to 10 nm. MWCNTs have a larger diameter, in the range of 2 to 50 nm. CNTs may be functionalized with a targeting sequence. For instance, CNTs may be functionalized to target mitochondria and/or a specific cell type. Graphene derivatives also include two-dimensional carbon allotropes. Graphene derivatives may be designed to exhibit specific physicochemical properties, such as a high surface area and selected multifaceted surface properties. Graphene derivatives may be functionalized, e.g., surface functionalized, for targeted delivery to mitochondria and/or a specific cell type. CNTs have been used successfully in hair colorants and cosmetic hair care formulations.

Nanodiamonds have been shown to provide high affinity to biomolecules, biocompatibility, and non- or low-cytotoxicity. Nanodiamonds, quantum dots, and magnetic nanoparticles may be conjugated with the compounds disclosed herein for targeted delivery. Certain MNP may be designed to encapsulate the compounds disclosed herein. QDs are generally formed of a semiconductive core layered by a shell designed to provide selected physical and chemical characteristics. MNPs are formed of a magnetic core, such as an iron oxide material core, and surface coating designed to improve stability and biocompatibility in physiological environments. Thus, MNPs and QDs are highly adaptable for their selected use.

Liposome based delivery vehicles are typically formed of enclosed spherical vesicles composed of a lipid bilayer with an internal bilayer and internal aqueous core region. The liposome may have a unilamellar or multilamellar structure. Liposomes may be designed to have a surface chemistry effective for targeted delivery and/or controlled release delivery. For instance, engineered liposomes have shown improved cellular update and accumulation of a delivery compound in the mitochondria. Antioxidants, such as carotenoids, CoQ10, lycopene and agents such as vitamin A, E, and K, may be incorporated into liposomes to amplify physical and chemical stability. Liposomes may be formulated with phosphatidylcholine to provide moisturizing properties to skin and hair care products. Vegetable phospholipids and soya phospholipids may be used with topical formulations for their high content of esterified essential fatty acids. For example, when applied with an active agent, the barrier function of the skin is increased and water loss is decreased. It has been shown that certain liposomes have an effect on wrinkle reduction, decreasing efflorescence in acne treatment, and increasing skin smoothness.

Niosomes are vesicles having a bilayer structure composed of self-assembled hydrated nonionic surfactants. Niosomes may have cholesterol incorporated into their lipids or may be free of cholesterol. Niosomes may be formulated as multilamellar or unilamellar structures encapsulating the compounds disclosed herein by a membrane formed when the surfactant macromolecules are organized as a bilayer. Exemplary nonionic surfactants include spans, tweens, brijs, alkyl amides, sorbitan ester, crown ester, polyoxyethylene alkyl ether, and steroid-linked surfactants. Niosomes may encapsulate the compounds disclosed herein, providing prolonged systemic circulation and enhanced penetration into target tissue. Niosomes in cosmetic and skin care applications provide skin penetration, increased stability of entrapped ingredients, and improved bioavailability of typically poorly adsorbed compounds. Niosomes may be designed for a target application by controlling the nature and structure of surfactants, membrane composition, and temperature of hydration, which influences size and shape of the particle. Specialized niosomes called proniosomes may be used. Proniosomes are nonionic based surfactant vesicles which are hydrated immediately before use to yield aqueous noisome dispersions. To further enhance drug delivery, niosomes and proniosomes may be combined in a formulation.

Polymeric nanocarriers are generally formed of biodegradable polymers. The polymeric nanocarriers may be reservoir type (nanocapsules in which compounds are dissolved/distributed in the core of the polymer), matrix type (nanospheres, in which compounds are entrapped in the polymer matrix), and combinations thereof. Polymeric nanocarriers may offer the benefits of low toxicity, easy modification (for example, for targeted delivery), high drug loading capacity, small size, good aqueous solubility, and biocompatibility. Exemplary polymeric nanocarriers for mitochondrial targeting include hydrophilic block polymer, such as polyethylene glycol (PEG), poly E-caprolactone (PCL). Other nanoparticles disclosed herein may be modified for mitochondrial targeting, such as by including surface hydrophilic block polymers (e.g., PEGylation). Polymerosomes are artificial vesicles formed of self-assembling block copolymer amphiphiles. Polymerosomes typically have a hydrophilic core and lipophilic bilayer. Polymerosomes are highly customizable. Drug encapsulation and release capabilities may be controlled by forming the polymerosome with block copolymers that are biodegradable and/or responsive to stimuli. The composition and molecular weight of the polymerosome may be selected to control properties, such as, response to stimuli, membrane thickness, permeability, flexibility, and size (polymerosomes may be designed to have a radius from 50 nm to 5000 nm or more). Polymerosomes provide benefits, such as, improved skin elasticity and skin cell activation energy enhancement.

Cubosomes are nanostructured particles formed from self-assembled liquid crystalline particles of aqueous lipids and surfactants. Cubosomes are formed of a bicontinuous liquid phase, enclosing two separate vesicles of aqueous formulations divided by a surfactant-controlled bilayer in a strongly packed structure. Cubosomes may be designed as a honeycombed structure having more than two separate vesicles. Release of each vesicle may be separately controlled. Cubosomes may provide benefits, such as, providing controlled and/or targeted release of the compounds, possessing lipid biodegradability, and having a high internal surface area with different drug-loading modalities.

Micelles are colloidal aggregates that are generally amphiphilic in nature, having a hydrophilic head and hydrophobic tail. The size and shape of micelle nanoparticles may be selected by varying the solution's isotonic strength, pH, temperature, and the nature of the amphiphilic molecule. Micelles may be utilized to improve uptake of the compounds disclosed herein in mitochondria. For instance, micelle formulations have been found to improve bioavailability of low absorption compounds, prevent mitochondrial swelling (indicating less mitochondrial permeability transition pore (mPTP) opening and prevention of injury), and protect cells from nitrosative stress, “Curcumin Micelles Improve Mitochondrial Function in Neuronal PC12 Cells and Brains of NMRI Mice-Impact on Bioavailability” (Hagl, 2015) (incorporated herein by reference in its entirety for all purposes). Micelles may be functionalized with a targeting group, such as mitochondrial targeting TPP, MPP, or PEGyltaion. In some embodiments, micelle nanoparticles may be polymeric micelles.

Dendrimers are hyperbranched macromolecules which may be formed of sugars, amino acids, and/or nucleotides. Dendrimers are generally formed of a central core, repeated branches, and diverse peripheral groups. The peripheral groups may be designed or functionalized for a target application, such as targeted delivery. Benefits of dendrimers include the ability to provide drug encapsulation, high aqueous solubility, high retention time, biodegradability, specificity, low toxicity, and surface modification capabilities that may provide properties such as monodispersity, polyvalence, and stability. Dendrimers may be functionalized with a targeting group, such as TPP, MPP, or PEGylation. Dendrimers may be formulated to encapsulate the compound or conjugate to the compound.

Lipophilic cations are positively charged ions that can penetrate the plasma and mitochondrial membranes. The lipophilic cations tend to accumulate in the mitochondria. Thus, lipophilic cations, such as TPP, dequalinum, and rhodamine 123, may be utilized for mitochondrial-targeted therapeutic delivery. Delocalized lipophilic cations (DLC) have a strong mitochondrial targeting ability to cross the membrane and drive specific aggregation of attached moieties within the mitochondria of the cells. Thus, DLCs may be utilized as nanoparticle carriers and/or as surface modifications for targeted delivery. For instance, the compositions disclosed herein may be conjugated to a DLC, such as TPP, or encapsulated in another carrier having a TPP surface functionalization for targeted delivery.

Solid-lipid nanoparticles are sub-micron colloidal carriers in the range of 50 to 1000 nm, for example 50 to 500 nm or 50 to 100 nm. SLNs are generally formed of physiological lipid disseminated in water or a liquid surfactant solution having an oil-based or lipoidal core. SLNs may be prepared from complex glyceride mixtures, purified triglycerides, and waxes having phospholipid hydrophobic chains in the fat matrix. SLNs provide benefits such as small size, large surface area, high drug loading capacity, and the contact of phases at the interface. SLNs may be designed to provide controlled or sustained release of the compound. In cosmetics and pharmaceuticals, SLNs may provide increased penetration of the compounds disclosed herein through the skin. SLNs may have ultraviolet (UV) resistant properties, occlusive properties which can be used to increase skin hydration, and good stability coalescence due to their solid nature, which reduces mobility and leakage of the active molecules.

Nanostructured lipid carriers (NLC) are a form of lipid nanoparticle formed by blending solid lipids with spatially incompatible liquid lipid compositions, forming an amorphous solid. NLCs may be of the imperfect type, amorphous type, or multiple type. NLC particles typically range in size from 10 nm to 1000 nm. When formulated from biodegradable and physiological lipids, NLCs show very little toxicity. Thus, NLC formulations may provide the benefits of reduced systemic side effects and higher drug loading capacity. NLCs may be designed to have a biphasic drug release pattern. For example, a first compound release profile may be immediate or controlled release, and a second compound release profile may be controlled or delayed release. Like SLNs, NLCs may also provide increased penetration of the compounds disclosed herein through the skin, ultraviolet (UV) resistant properties, occlusive properties which can be used to increase skin hydration, and good stability coalescence.

Nanoemulsions are kinetically and thermodynamically stable dispersions of liquid formed from an oil phase and a water phase in combination with a surfactant. The nanoemulsions disclosed herein may be oil in water, water in oil, or bicontinuous formulations. Properties of nanoemulsions may be designed by controlling method of preparation. Nanoemulsions are typically dispersed phase, comprising small particles or droplets having low oil or water interfacial tension. Nanoemulsions are tupically formed of a lipophilic core surrounded by a monomolecular layer of phospholipids. Nanoemulsions provide benefits, such as, low viscosity, high kinetic stability, high interfacial area, high solubilization capacity, and increased rate of absorption. In cosmetic and pharmaceuticals, nanoemulsions may provide rapid penetration and active transport of active ingredients and hydration to the skin. Nanoemulsions may be formulated into foams, creams, sprays, or liquids.

Certain nanoparticle formulations, such as SLNs, nanoemulsions, liposomes, and niosomes, may be used in moisturizing formulations, providing humectants that retain moisture for a prolonged period of time.

Metal nanoparticles may be designed to have specific properties and may be shaped as nanospheres, nanoshells, nanoclusters, nanorods, nanostars, nanocubes, branched, and nanotriangles. Shape, size, and dielectric properties of metal nanoparticles may have an effect on resonance frequency. Metal nanoparticles may be designed to have high drug loading capacity and effectively penetrate the cell wall by controlling size, surface area, and crystallinity. Benefits of metal nanoparticles include acceleration of blood circulation, anti-inflammatory properties, antiseptic properties, improvising firmness and elasticity of skin, delaying aging, and vitalizing skin metabolism. Exemplary metal nanoparticles are gold, silver, and copper. Such metal nanoparticles have been shown to provide strong antifungal and/or antimicrobial properties. Another exemplary metal nanoparticle is titanium dioxide (TiO₂), which has been shown to provide protection from ultraviolet (UV) radiation. Metal nanoparticles may be designed to be inert in nature, highly stable, biocompatible, and noncytotoxic. More than one metal may be used to form metal nanocomposites with selected properties.

Nanospheres are spherical nanoparticles having a core-shell structure. The compounds may be encapsulated, conjugated, dissolved, or otherwise entrapped in the nanoparticle. The nanospheres may be crystalline or amorphous structures. The nanospheres may be biodegradable or nonbiodegradable. Exemplary biodegradable biospheres include gelatin, modified starch, and albumin nanospheres. One exemplary nonbiodegradable nanosphere is polylactic acid. In cosmetics and pharmaceuticals, nanospheres may be used to deliver the compounds disclosed herein into a deep layer of the skin more precisely and efficiently. Nanospheres have been shown to provide protection against actinic aging.

Peptide-based nanomaterials are biomolecules made up of several amino acids linked by peptide bond. Peptides may generally provide rapid clearance in the kidney due to enzymatic degradation. Peptide nanomaterials may provide several benefits including targeting and accumulating capacity, small size, case of production and customizability, and biocompatibility. Certain peptide nanomaterials may be designed to self-assemble into distinct shapes and sizes in response to environmental factors, such as temperature, pH, ionic strength, or molecular interaction between the host and peptide. Peptide nanoparticles may also be functionalized for targeted delivery. Functionalized peptides have been found to exhibit improved targetability and enhanced efficacy.

One exemplary peptide nanomaterial with mitochondrial-targeting ability is mitochondrial penetrating peptides (MPP). MPPs are cell penetrating peptides which can efficiently penetrate mitochondrial double membranes. MPPs are generally designed or selected to be positively charged peptides. Due to the strongly negative charge of mitochondrial membranes, positively charged peptides are capable of penetrating mitochondria. MPPs are described in more detail in “Mitochondrial targeted strategies and their application for cancer and other diseases treatment.” (Li, 2020), incorporated herein by reference in its entirety for all purposes. Thus, MPPs may be utilized as nanoparticle carriers and/or as surface modifications for targeted delivery. For instance, the compositions disclosed herein may be conjugated to an MPP or encapsulated in another carrier having an MPP surface functionalization for targeted delivery.

The formulations disclosed herein may comprise one or more skin penetration enhancer. Generally, small and moderately lipophilic molecules are likely to penetrate the skin barrier. Other compounds may require a suitable skin penetration enhancer to penetrate the barrier by either diminishing the barrier properties of the skin or actively driving movement of compounds across the skin with the input of external energy. Skin penetration enhancers are described in “Penetration Enhancement of Topical Formulations,” (Ng. 2018) and “Transdermal Delivery Systems in Cosmetics,” (Kim, 2020), each of which is incorporated herein by reference in its entirety for all purposes.

The nanoparticle delivery carriers disclosed herein may provide skin penetration enhancement for the compounds. The formulations disclosed herein may additionally or alternatively contain one or more chemical or physical skin penetration enhancers. Exemplary chemical skin penetration enhancers include alcohols, such as, ethanol and glycol, sulfoxides, such as, dimethyl sulfoxide, laurocapram, pyrrolidones, dimethyl isosorbide, isopropyl myristate, propylene glycol, oleic acid, eucalyptol, water/aqua (hydration), surfactants, urea, fatty acids, fatty alcohols, and terpenes and/or terpenoids. Exemplary physical skin penetration enhancers include rollers, scrapers, scrubbers, exfoliators, microdermabrasion needles, iontophoresis devices, electroporation devices, ultrasound devices, such as sonophoresis, thermal ablation, magnetophoresis, photomechanical waves, electron beam irradiation, and low light therapy devices, such as light emitting diode (LED) sources. Two or more skin penetration enhancers may be used in the formulation synergistically.

Chemical skin penetration enhancers may be included in dermatological, transdermal, cosmetic, and pharmaceutical products to enhance the dermal absorption of drug compounds. Chemical enhancers may enable or improve solubility, penetration, and/or absorption of the compounds disclosed herein. Exemplary chemical enhancers are described below.

Water (aqua) generally increases fluidity of the composition, providing higher permeability. Additionally, water hydrates the skin barrier, altering skin lipids and/or proteins for improved permeation. Hydrating compounds, such as glycerol and urea, may promote transdermal permeation by facilitating hydration of the stratum corneum and forming hydrophilic diffusion channels within the barrier.

Alcohol solvents, such as ethanol and propylene glycol, may provide penetration enhancement by increasing fluidity of the compound and also act as good solvents. Degree of permeation may be selected by controlling alkyl chain length of fatty alcohols.

Surfactants generally solubilize lipophilic agents, including active ingredients of the formulation as well as lipids within the stratum corneum. Thus, surfactants may enhance skin permeability by partitioning into the epithelial cell membranes and disrupting the packing of membrane lipids, forming structural defects that reduce membrane integrity. The effect of the surfactant on skin permeation may be designed by selecting concentration and type of surfactant. The surfactant may be anionic, cationic, or nonionic. Anionic surfactants may be selected for skin or hair applications because they interact with keratin and lipids. Cationic surfactants may be selected for skin applications because they interact with skin proteins via poler interactions. Non-ionic surfactants are generally less irritating to the skin and have better tolerability.

Fatty acids may increase percutaneous drug absorption. Long-chain fatty acids, which are carboxylic acids with typically long, unbranched aliphatic tails, have been demonstrated to increase percutaneous drug absorption as an effect of alkyl chain length. Low molecular weight alkanols may act as solubilizers to enhance the solubility of the compound in the fatty matric of the stratum corneum. Polyunsaturated fatty acids, such as linoleic, alpha-linoleic, and arachidonic acids, may enhance the skin permeation. One exemplary fatty acid chemical penetration enhancer is oleic acid. Oleic acid provides increased fluidity and reduced resistance toward the permeation of molecules.

Terpenes are hydrocarbons commonly found in plant extracts. Terpenoids are terpenes containing additional functional groups. One group of terpenes that may provide penetration enhancement are oxygen-containing terpenes. Exemplary oxygen containing terpenes include menthol, thymol, carvacrol, menthone, and cineole. Such terpenes may enhance penetration in a similar mechanism as alcohols. However, terpenes may be considered natural products. Benefits of terpenes include high percutaneous ability with minimal irritancy and toxicity.

The compounds of the disclosure may be formulated with a mitochondria-targeting agent. Exemplary mitochondrial targeting agents include antibodies, polymeric functional moieties, such as PEG, lipophilic cations, such as triphenylphosphine (TPP), and peptides such as mitochondrial targeting peptides (MPP).

The compounds of the disclosure may alternatively contain as pharmaceutically and/or cosmetically acceptable carriers substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. For solid compositions, conventional nontoxic pharmaceutically and/or cosmetically acceptable carriers can be used which include, for example, pharmaceutical grades of mannitol, lactose, starch, magnesium stearate, sodium saccharin, talcum, magnesium carbonate, and the like.

Compositions of the disclosure can also be formulated as a solution, microemulsion, or other ordered structure suitable for high concentration of active ingredients. The carrier can be a solvent or dispersion medium containing, for example, water, ethanol, polyol (for example, glycerol, propylene glycol, and liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants.

In certain embodiments, compound(s) and compositions of the disclosure are administered in a time-release formulation, for example in a composition which includes a slow release polymer. Such compositions can be prepared with carriers that will protect against rapid release, for example a controlled release vehicle such as a polymer, microencapsulated delivery system or bioadhesive gel. Prolonged delivery, in various compositions of the invention can be brought about by including in the composition agents that delay absorption, for example, aluminum monosterate hydrogels and gelatin. When controlled release formulations is desired, controlled release binders suitable for use in accordance with the invention include any biocompatible controlled-release material which is inert to the active agent and which is capable of incorporating the biologically active agent. Numerous such materials are known in the art.

Formulations suitable for topical administration include solutions, oils, creams, emulsions, and gels containing, in addition to the active ingredient, such carriers as are known in the art. Topical formulations may be pharmaceutical or cosmetic formulations. In some embodiments, the compound is formulated as a shampoo, conditioner, spray, cream, gel, balm, body wash, soap, lotion, or make-up.

The compounds of the disclosure and compositions of the disclosure can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Suitable unit doses, i.e., effective amounts, may be determined during clinical trials designed appropriately for each of the conditions for which administration of a chosen compound is indicated and will, of course, vary depending on the desired clinical endpoint. Extemporaneous injection solutions and suspensions can be prepared from sterile powders, granules, and tablets. The requirements for effective pharmaceutically acceptable carriers for injectable compositions are well known to those of ordinary skill in the art. See Pharmaceutics and Pharmacy Practice, J.B. Lippincott Co., Philadelphia, Pa., Banker and Chalmers, Eds., 238-250 (1982) and ASHP Handbook on Injectable Drugs, Toissel, 4^{th} ed., 622-630 (1986).

Additionally, formulations suitable for rectal administration may be presented as suppositories by mixing with a variety of bases such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

The compositions disclosed herein may be associated with a variety of natural products, and examples of such products are set out below. The compositions disclosed herein may be formulated as a natural product. The compositions disclosed herein may be administered in combination with a natural product. The compositions disclosed herein may be incorporated in a natural product. These natural products may be comprised of formulations or compositions disclosed throughout this disclosure.

Natural products may be or comprise products for commercial purposes, and may refer to dietary supplements, and foods, e.g., food, food supplements, medical food, food additive, nutraceutical, or drink, produced from natural sources. Natural products may have pharmacological or biological activity that may be of therapeutic benefit, e.g., in treating disease or conditions. Natural products may be included in traditional medicines, treatments for cosmetological purposes, cosmetics, and spa treatments. A natural product referred to herein may comprise any one or more of the components described as a natural product to be incorporated into a composition or formulation comprising one or more other components, e.g., excipients. The preparation or formulation referred to as a natural product may comprise a natural product defined herein and one or more additional components or ingredients. Any of the compositions, preparations, or formulations discussed throughout this disclosure may be or comprise one or more natural products.

One skilled in the art will appreciate that suitable methods of administering a compound of the disclosure to a patient are available, and, although more than one route can be used to administer a particular compound, a particular route can provide a more immediate and more effective reaction than another route.

### EXAMPLES

The function and advantages of these and other embodiments can be better understood from the following examples. These examples are intended to be illustrative in nature and are not considered to be limiting the scope of the invention.

### Example 1: Identification of Compounds from Emblica Extract

To identify compounds present in Emblica, an Emblica extract was prepared and analyzed by HPLC.

In this example, 40.9 grams of caplets containing E. officinalis extract (Himalaya Drug Company, Sugar Land, TX Lot 112000924; each caplet contains 250 mg fruit extract (45% tannins) and 350 mg powder stem (2% tannins)) was stirred with 250 ml methanol for 6 hours at room temperature. The resulting solution was filtered to remove insoluble material and the methanol layer was stripped using a Rotovap (fraction 1). The solid material resulting from the filtration step was stirred with an additional 250 ml of methanol for 4 hours at room temperature, filtered through to remove insoluble material and a filter funnel and the methanol layer was stripped using a Rotovap (fraction 2). Fractions 1 and 2 were combined to obtain 8.25 grams of a dark, hydroscopic solid.

450 mg of solid isolated as described above was dissolved in 2 ml of methanol. The resulting solution was sonicated until all contents were in solution and subject to preparative HPLC purification using a ACCQPrep instrument (Teledyne ISCO, Lincoln, NE) with a Phenomenex Gemini® 5 μM NX-C18 110A 150×4.6 mm liquid chromatography column (Phenomenex, Torrance, CA). HPLC was carried out with a two solvent gradient as described in the Table 3 below.

TABLE 3
HPLC Gradient.

| Gradient | % | % | |
|---|---|---|---|
| Time | Solvent | Solvent | Flow rate |
| (min) | A | B | (ml/min) |
| 0 | 0 | 100 | 42.5 |
| 5 | 0 | 100 | 42.5 |
| 20 | 90 | 10 | 45.2 |
| 24 | 90 | 10 | 42.5 |
| 26 | 5 | 95 | 42.5 |
| 28 | 5 | 95 | 42.5 |
| 29 | 50 | 50 | 42.5 |
| 43 | 50 | 50 | 42.5 |

Solvent A-acetonitrile;
Solvent B-water
Water purified through a PureLab ® Ultra water purification system (ELGA LabWater, Woodridge, IL).

The collected fractions were stripped and fractions transferred into eleven separate vials. 1 mg of each fraction was taken and dissolved in 1 ml of methanol in a 1.5 ml vial. The resulting solution was sonicated until all contents were in solution and subject to analytical HPLC analysis using an Agilent 1100 Series instrument (Agilent Technologies, Santa Clara, CA) with a Phenomenex Gemini® 5 μM NX-C18 110A 150×4.6 mm liquid chromatography column (Phenomenex, Torrance, CA). HPLC was carried out with a two solvent gradient as described in the Table 4 below.

TABLE 4
HPLC Gradient.

| Gradient | % | % | |
|---|---|---|---|
| Time | Solvent | Solvent | Flow rate |
| (min) | A | B | (ml/min) |
| 0 | 0 | 100 | 1 |
| 5 | 0 | 100 | 1 |
| 20 | 97.5 | 2.5 | 1 |
| 24 | 90 | 10 | 1 |
| 26 | 5 | 95 | 1 |
| 28 | 5 | 95 | 1 |
| 29 | 100 | 0 | 1 |
| 43 | 100 | 0 | 1 |

Solvent A-acetonitrile with 0.1% trifluoroacetic acid (TFA)
Solvent B-water with 0.1% TFA
Water purified through a PureLab ® Ultra water purification system (ELGA LabWater, Woodridge, IL).

To identify specific compounds in the Emblica extract the standards shown in Table 5 were used.

TABLE 5
Emblica Extract Compound Identification Standards.

| | | | Retention |
|---|---|---|---|
| | | | Time |
| Common name | Cas # | Source | (min) |
| Gallic Acid | 149-91-7 | Acros | 6.88 |
| | | Organics | 7.22 |
| vanillic acid | 121-34-6 | Alfa Aesar | 27.64 |
| chlorogenic acid | 327-97-9 | TCI America | 28.61 |
| caffenic acid | 331-39-5 | TCI America | 28.65 |
| Syringic acid | 530-57-4 | Acros | 28.86 |
| | | Organics | |
| tp coumaric acid | 501-98-4 | TCI America | 29.52 |
| quercetin | 849061-97-8 | Acros | 30.63 |
| | | Organics | |
| Vitamin C | 50-81-7 | Sigma | 2.36 |
| | | Aldrich | 3.3 |

Standards were prepared by dissolving 1 mg of the standard in 1 ml methanol, sonicating the solution, and adding the solution to a 2 ml vial. Samples were subject to analytical HPLC as described above and to validate purity. A combined standard solution was created by adding 100 ul of each standard to a separate 2 ml vial. The standards were used to identify components in the Emblica extract.

The following compounds (Table 6) were identified in the Emblica extract prepared and analyzed as described above.

TABLE 6
Compounds identified in emblica extract.

| | | Estimated amount |
|---|---|---|
| Common name | Cas # | in emblica tablets |
| Gallic Acid | 149-91-7 | 70% |
| vanillic acid | 121-34-6 | Less than 2% |
| chlorogenic acid | 327-97-9 | Less than 2% |
| caffenic acid | 331-39-5 | Less than 2% |
| Syringic acid | 530-57-4 | Less than 2% |
| tp coumaric acid | 501-98-4 | Less than 2% |
| quercetin | 849061-97-8 | Less than 2% |
| Vitamin C | 50-81-7 | 20% |

In addition, approximately 20 other compounds were present but have not yet been identified.

### Example 2: Prophetic Example Showing In Vivo Energy and Vitality Improving Effects of Application of Emblica Extract

Male and female subjects aged between 35 and 70 years showing aging in the form of visible eye wrinkles without further specific inclusion criteria will be evaluated for the study. The subjects will complete a baseline questionnaire of 10 closed questions with predefined options to be selected. The questionnaire will request information relating to baseline energy levels and vitality. Other baseline parameters will be measured for skin roughness (Ra, Rz) by DermaTOP (three-dimensional imaging of surface structure), skin hydration by Corncometer (Courage & Khazaka, Cologne, Germany) (electrical capacitance measurement), and skin elasticity by Cutometer (optical measurement of skin displacement during 300 mbar suction).

The subjects will apply Emblica extract (2-5 drops of Emblica extract oil) or placebo twice daily topically to one side of the face for approximately 12 weeks. The study will be performed in a split-face design. Anti-wrinkling properties will be measured periorbitally in the region of crowfeet. Skin moisturizing effects will be evaluated on the bones of the cheeks. Effect of the Emblica extract will be compared to the reference product or placebo.

Subjects will complete a final questionnaire at completion of the study. The final questionnaire will request information relating to baseline energy levels and vitality. Skin roughness (Ra, Rz), skin hydration, and skin elasticity will also be measured at the completion of the study. Baseline results will be compared to final results.

It is expected that administration of Emblica extract will improve energy levels and vitality. It is also expected that administration of Emblica extract will improve aging-associated parameters including skin roughness, skin hydration, skin elasticity, and additional qualitative parameters measured by the questionnaire.

### Example 3: Restoration of Mitochondrial DNA Depletion and Function and Suppression of Inflammation by Emblica Extract In Vivo

In these experiments, the shaved dorsal skin of 8-9 weeks old female C57BL/6 control and mtDNA-depleter mice (expressing D1135A-POLG1) were treated topically with 200 μl of 50 mg/ml Emblica extract ointment (prepared as described in the Methods section) or a corresponding amount of the control ointment (lacking Emblica extract) daily beginning 1 week prior to the start of dox administration (200 mg/kg diet only) and continuing daily applications for 16 weeks (112 days).

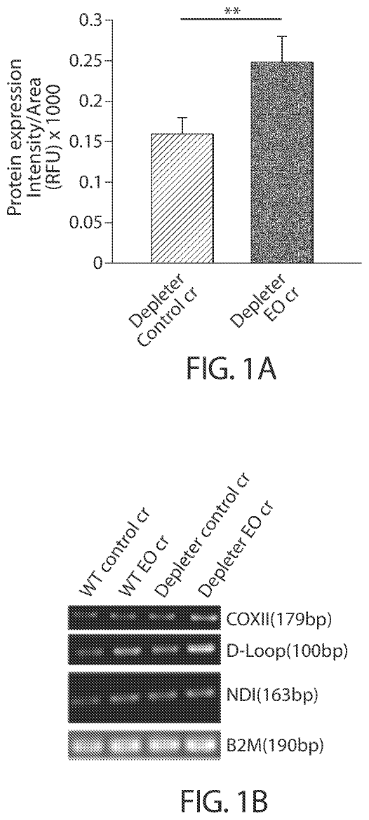

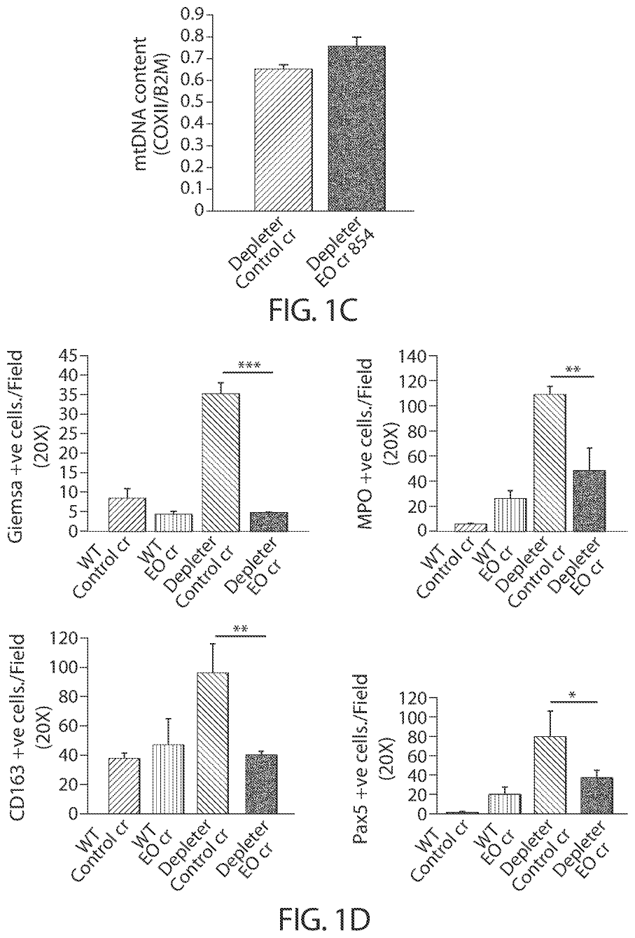

The effect of Emblica extract treatment on reversal of mtDNA function was examined by staining paraffin embedded dorsal skin sections mtDNA-depleter mice treated with Emblica extract ointment or control ointment with Oxphos complex IV antibody (COXII) (n=3). A statistically significant increase in COXII staining in mtDNA-depleter skin treated with Emblica extract as compared to control treatment was observed (FIG. 1A). In addition, RT-PCR analysis of mtDNA encoded genes showed upregulation in mtDNA-depleter skin treated with Emblica extract as compared to control treatment (FIG. 1B). Finally, an increase in mtDNA content was observed in skin samples from mtDNA-depleter mice treated with Emblica extract as compared to control treatment (FIG. 1C).

As described herein, the skin of mtDNA depleter mice showed increase in dermal and peri-appendageal mixed inflammatory cells, including mast cells, neutrophils and lymphocytes. After treatment with Emblica extract, the skin of mtDNA depleter mice showed a statistically significant decrease in dermal and peri-appendageal inflammatory cells (FIG. 1D) including mast cells (Giemsa+ve positive cells; p=3.56E-07), granulocytes (MPO+ve cells; P=0.002), macrophages and histiocytes (CD163+ve cells; p=0.007), and B lymphocytes (Pax-5+ve cells; p=0.042). Decreased expression of inflammatory genes in the skin samples of mtDNA-depleter mice treated with Emblica extract as to mtDNA-depleter mice treated with control ointment was also observed (data not shown).

### Example 4: Restoration of Mitochondrial DNA Depletion, as Evidenced by Reversal of Wrinkled Skin and Loss of Hair by Fucus Extract In Vivo

To investigate the ability of other agents to restore mitochondrial DNA and function, a Fucus extract was examined. Reversal of mitochondrial DNA depletion is evidenced by reversal of wrinkled skin and loss of hair. However, reversal of mitochondrial DNA depletion has a therapeutic effect on other indications.

In these experiments, the shaved dorsal skin of 8-9 weeks old female C57BL/6 control and mtDNA-depleter mice (expressing D1135A-POLG1) were treated topically with 200 ul of 50 mg/ml Fucus extract ointment (prepared as described in the Methods section) or a corresponding amount of the control ointment (lacking Fucus extract) daily beginning 1 week prior to the start of dox administration (200 mg/kg diet and 2 mg/mL in 5% sucrose water) and continued for 51 days (n=4 for each group).

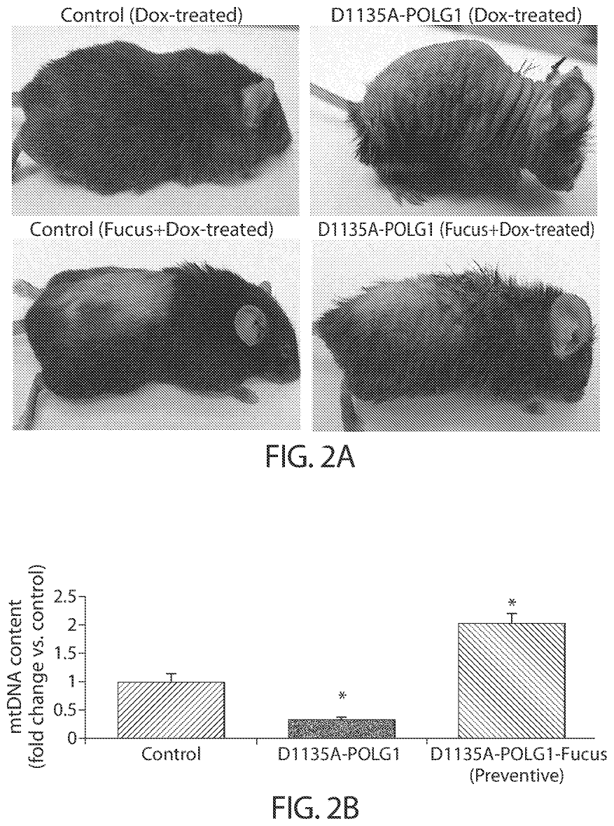

The results are shown in FIG. 2A. Consistent with previous results, dox administration to mtDNA-depleter mice resulted in significant hair loss and skin wrinkling phenotype. Administration of Fucus extract partially reversed the hair loss and wrinkled skin phenotype as compared to mtDNA-depleter mice without Fucus extract treatment (dox administration alone). Control mice showed no effects when treated with dox alone or dox in combination with Fucus extract.

### Example 5: Restoration of Mitochondrial DNA Content by Fucus Extract In Vivo

The effect of Fucus extract treatment on mitochondrial DNA content was also examined. Skin samples from the animals of Example 4 were collected and analyzed for mitochondrial DNA content and the results are shown in FIG. 2B. As shown in FIG. 2B, dox administration to mtDNA-depleter mice resulted in significant decrease in mitochondrial DNA content as compared to control mice. Administration of Fucus extract fully restored mitochondrial DNA content. These results show that the beneficial effects of Fucus extract on reversing the hair loss and wrinkled skin phenotype in mtDNA-depleter mice is correlated with preservation of mitochondrial DNA content.

### Example 6: Expression of Mitochondrial Biogenesis Regulatory Proteins In Vitro

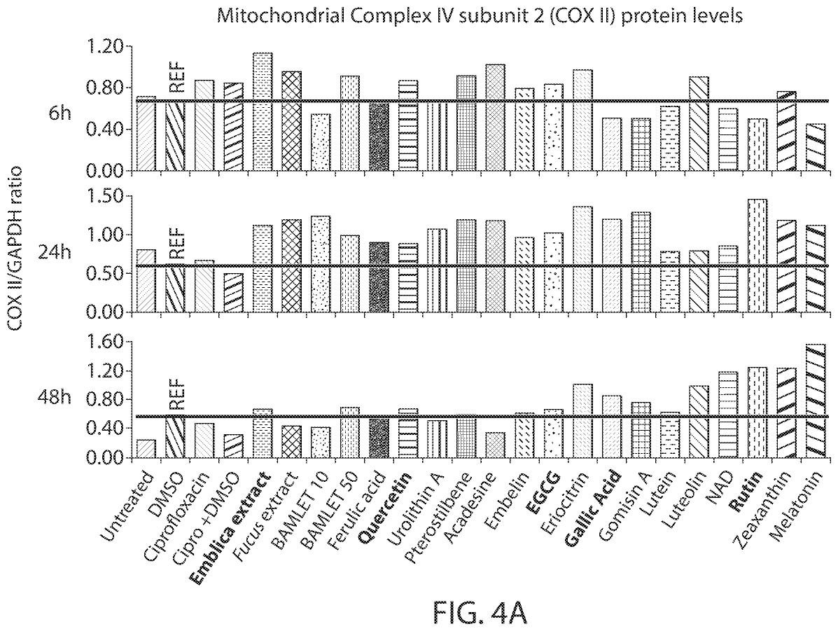

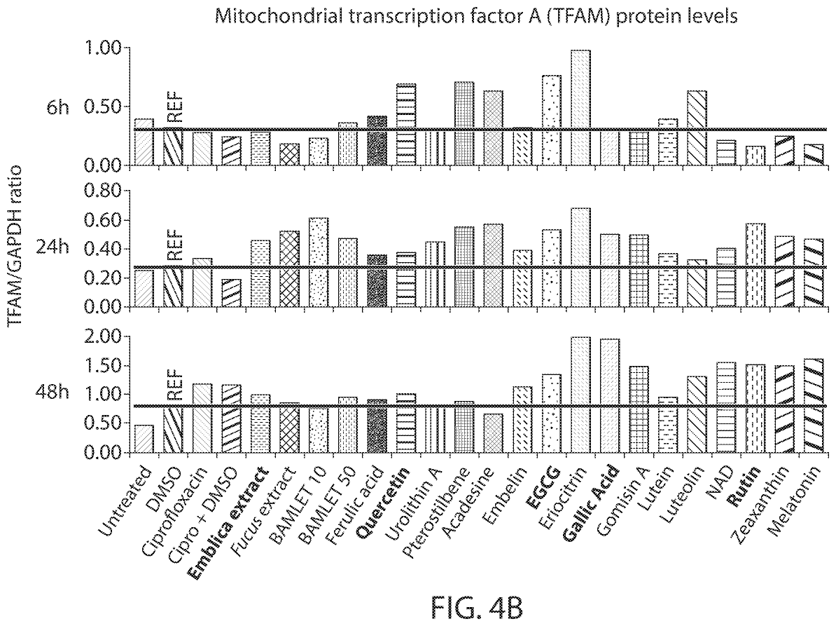

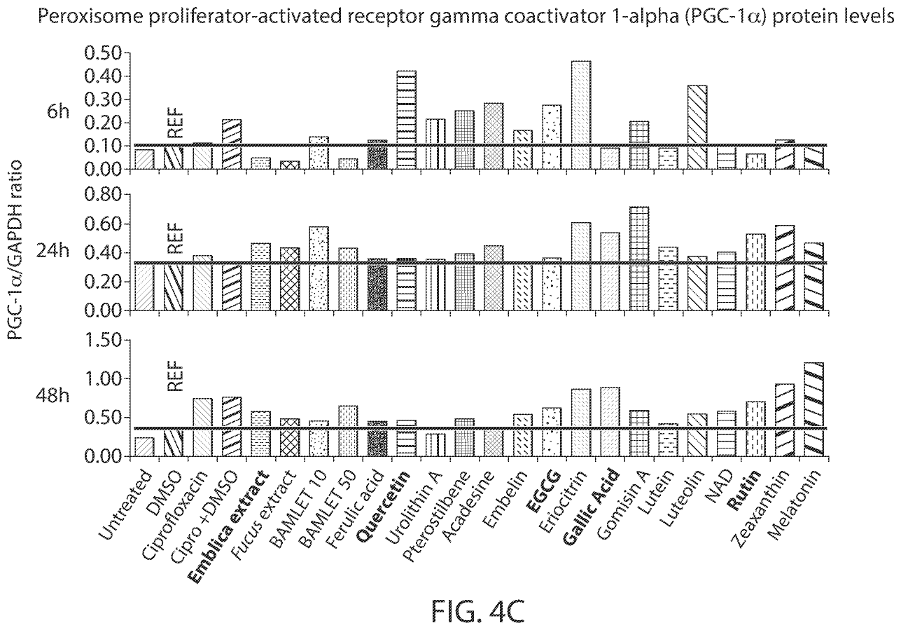

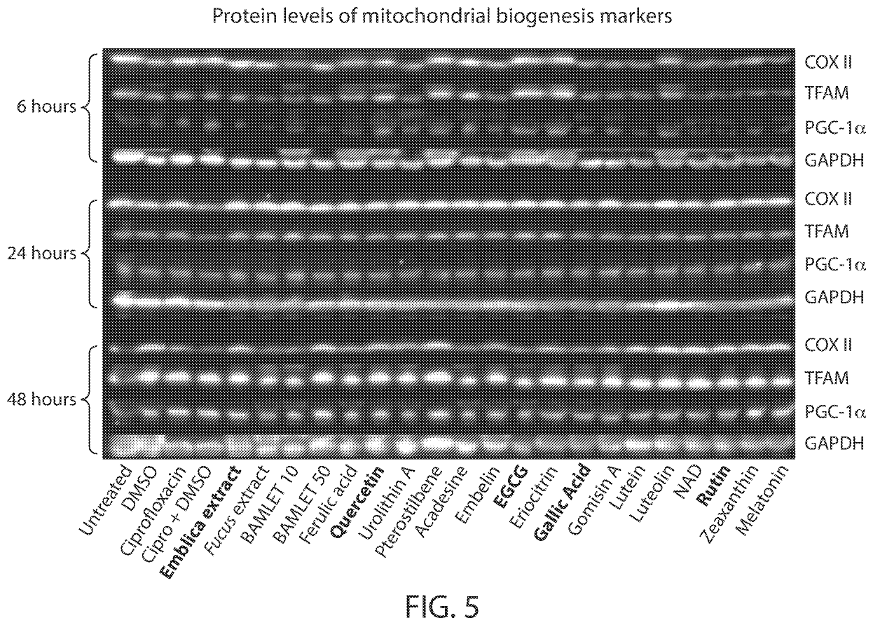

Emblica extract, components thereof, Fucus extract, and related compounds were tested in vitro for induced expression of mitochondrial biogenesis regulatory proteins including mitochondrial complex IV subunit 2 (COXII), mitochondrial transcript factor A (TFAM), and peroxisome proliferator-activated receptor gamma coactivator 1-alpha (PGC-1a) at timepoints of 6 hours, 24 hours, and 48 hours post-administration. The results are presented in the graphs of FIGS. 4A-4C and the gel-electrophoresis image of FIG. 5.

Many of the tested compositions showed increased expression of the proteins as compared to a DMSO reference. Expression generally increased with increasing time.

### Example 7: Extended Expression of Mitochondrial Biogenesis Regulatory Proteins In Vitro

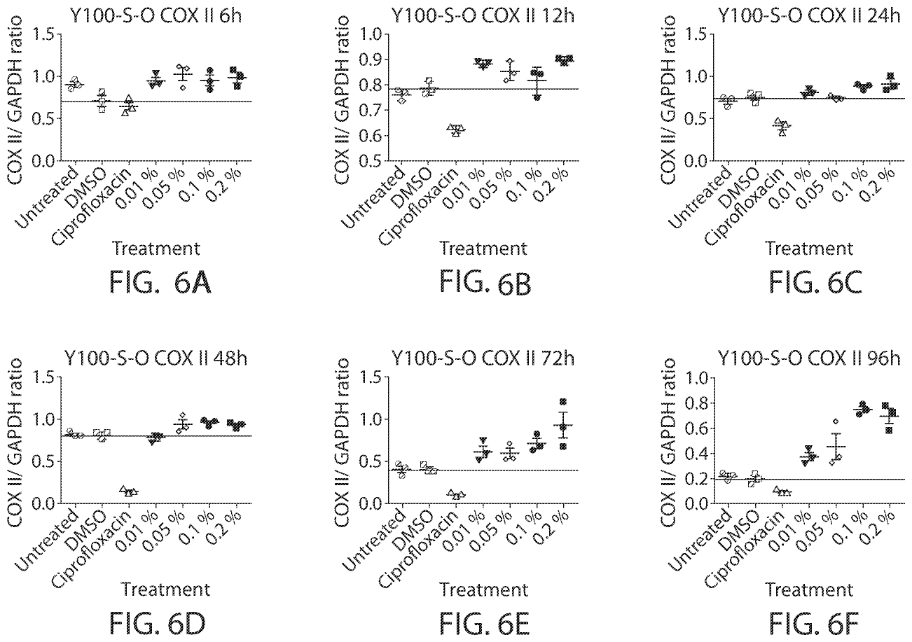

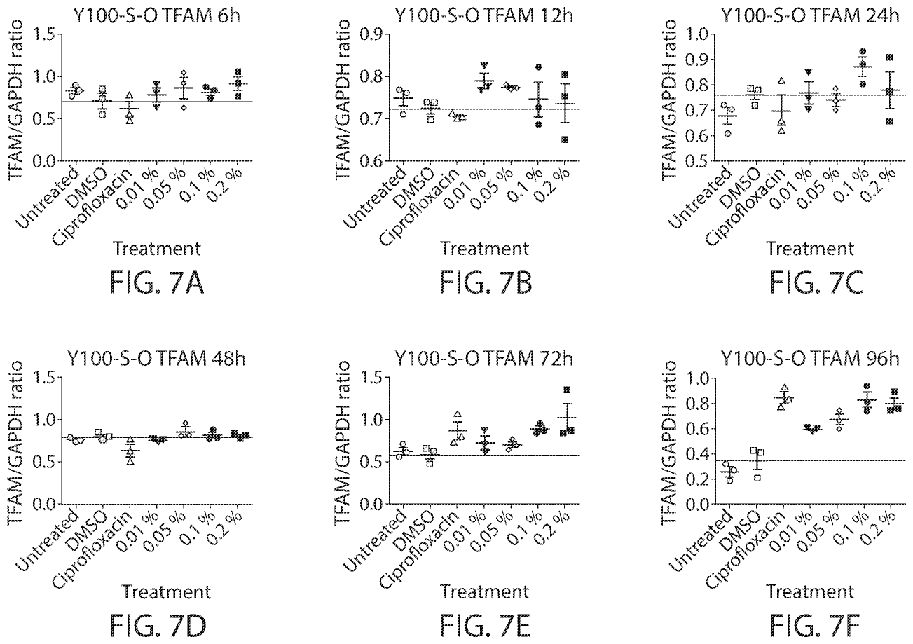

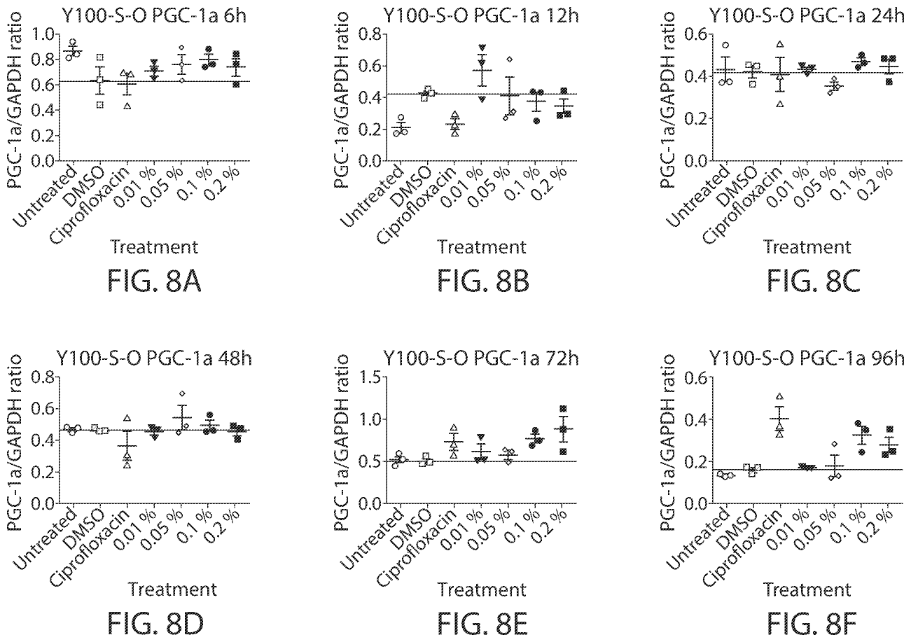

Emblica extract was tested in vitro for induced expression of mitochondrial biogenesis regulatory proteins including mitochondrial complex IV subunit 2 (COXII), mitochondrial transcript factor A (TFAM), and peroxisome proliferator-activated receptor gamma coactivator 1-alpha (PGC-1a) at timepoints of 6 hours, 12 hours, 24 hours, 48 hours, 72 hours, and 96 hours post-administration, generally as described in previous examples. Formulations of 0.01%, 0.05%, 0.1% and 0.2% Emblica extract were tested. The results are presented in the graphs of FIGS. 6A-6F (COXII expression), 7A-7F (TFAM expression), and 8A-8F (PCG-1a expression). The general trend is that certain formulations induced greater expression after 96 hours than at earlier timepoints.

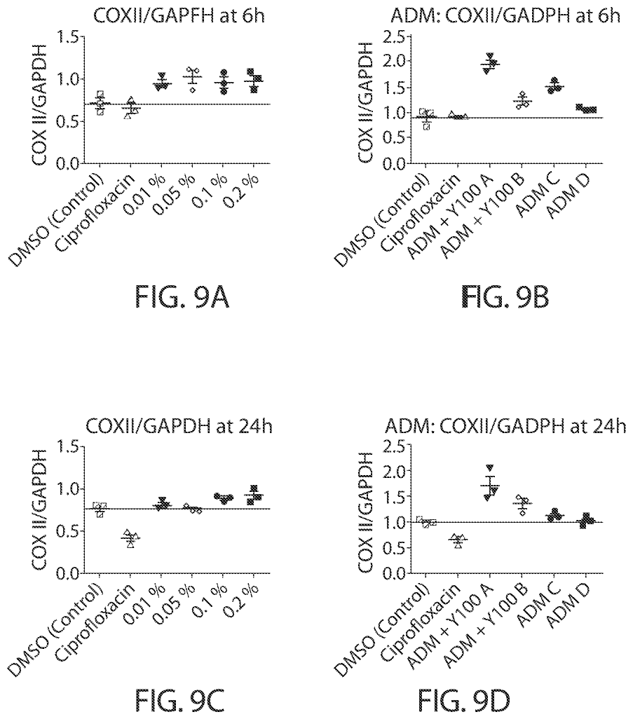

Emblica extract formulated with a nanoparticle delivery carrier was tested in vitro for induced expression of COXII and compared to a non-encapsulated composition at timepoints of 6 hours and 24 hours post-administration. The results are presented in the graphs of FIGS. 9A-9D. Specifically, FIG. 9A shows COXII expression at 6 hours after administration of a non-encapsulated composition. FIG. 9B shows COXII expression at 6 hours after administration of a nanoencapsulated composition. FIG. 9C shows COXII expression at 24 hours after administration of a non-encapsulated composition. FIG. 9D shows COXII expression at 24 hours after administration of a nanoencapsulated composition.

As shown in the data presented in FIGS. 9A-9D, all Emblica extract formulations showed an increase in COXII expression and certain nanoencapsulated formulations showed a greater increase in COXII expression.

### Example 8: Expression of Mitochondrial Biogenesis Regulatory Proteins In Vitro

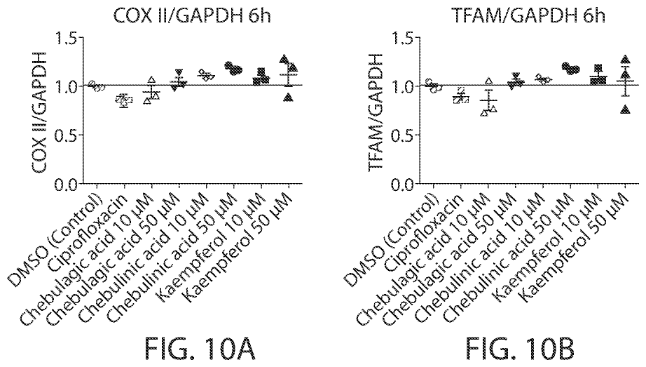

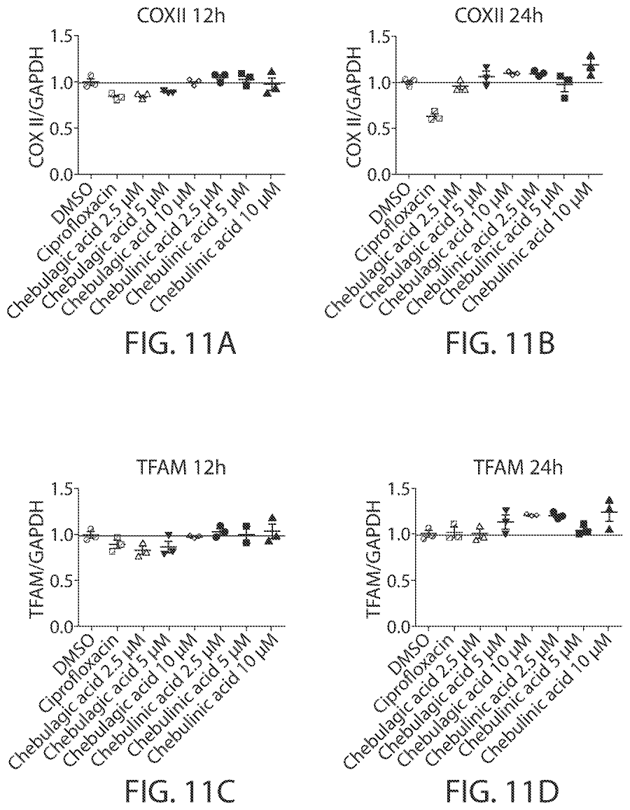

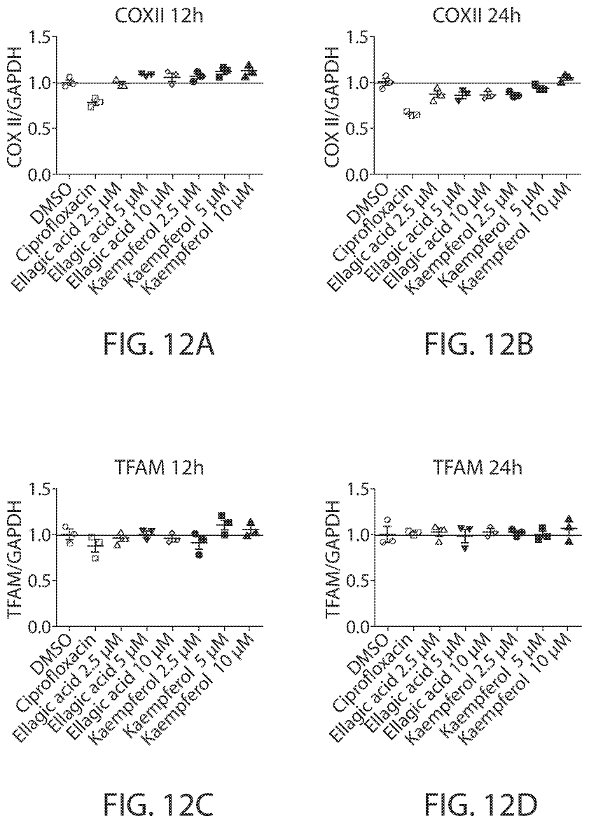

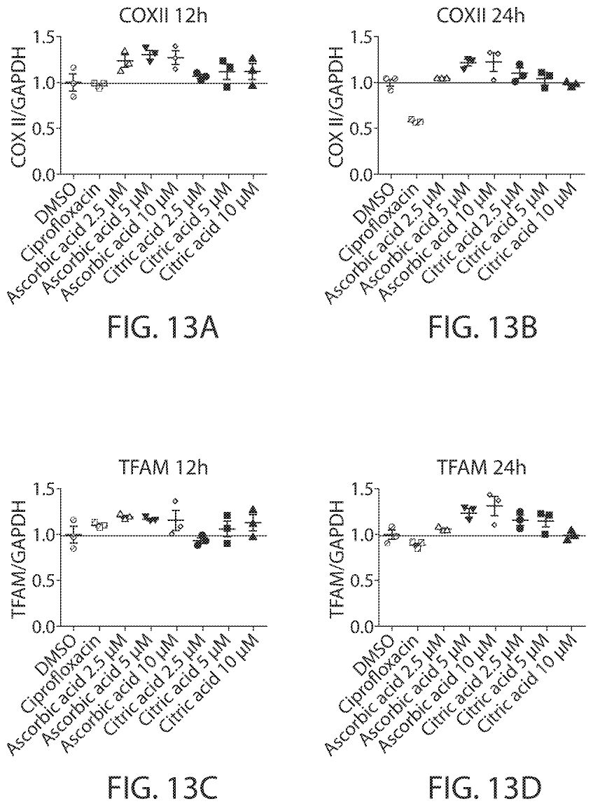

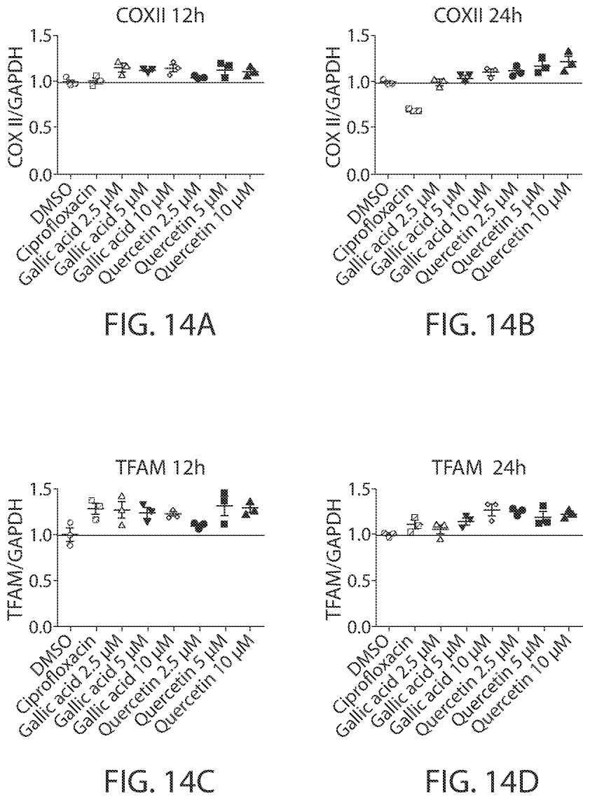

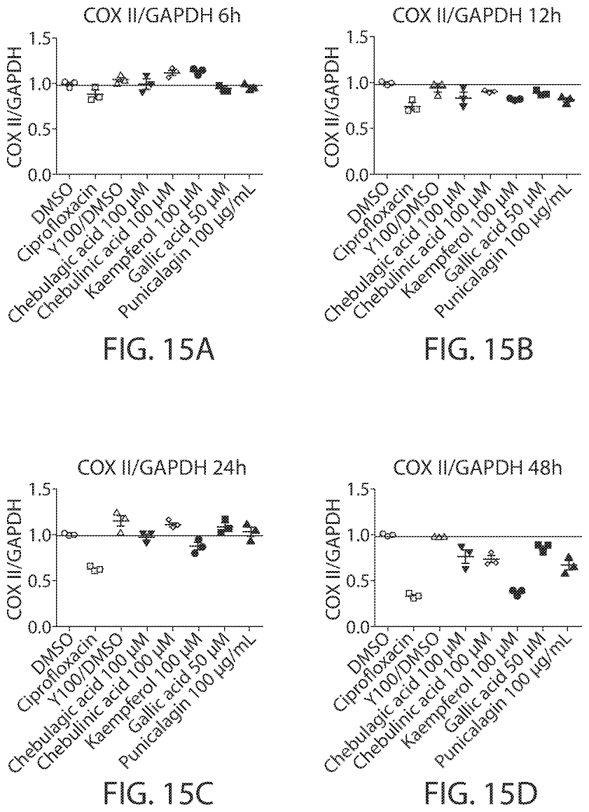

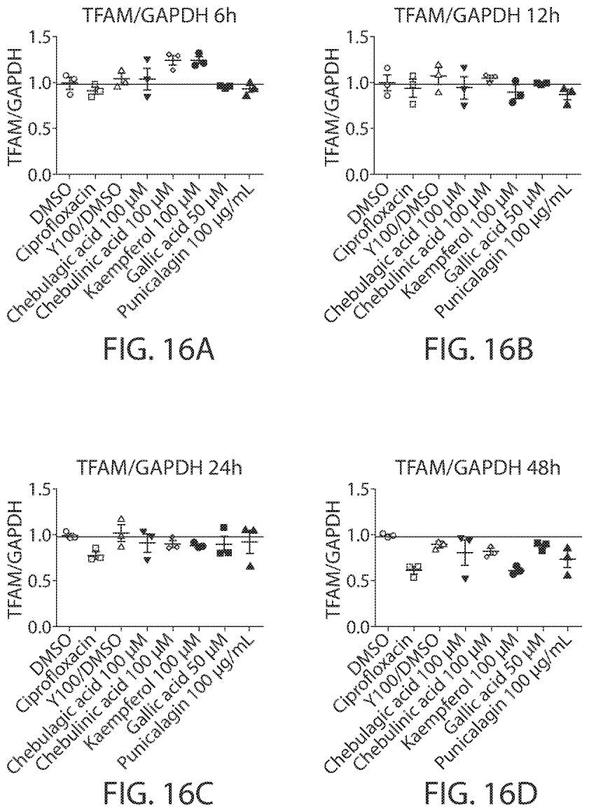

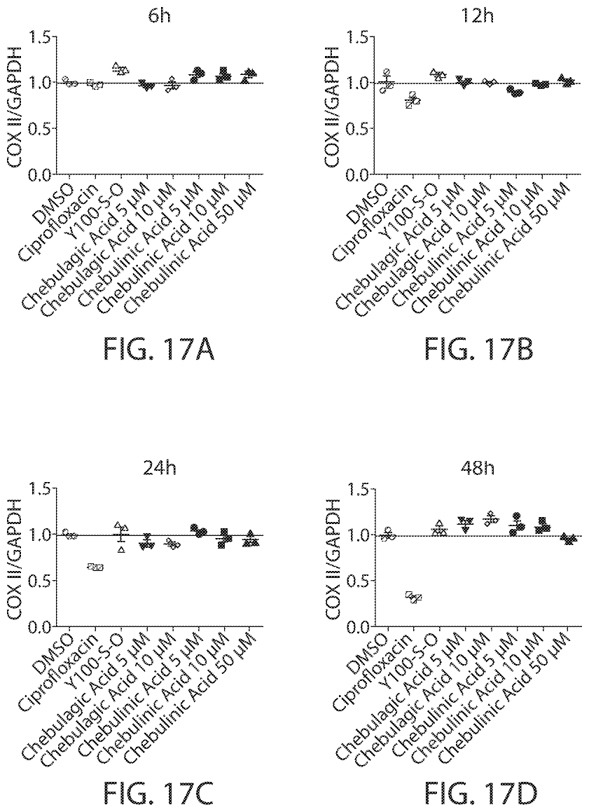

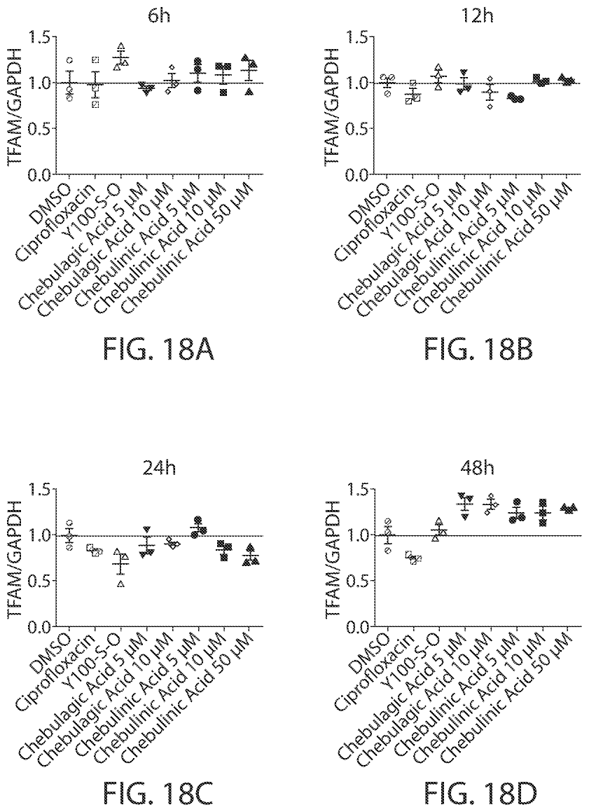

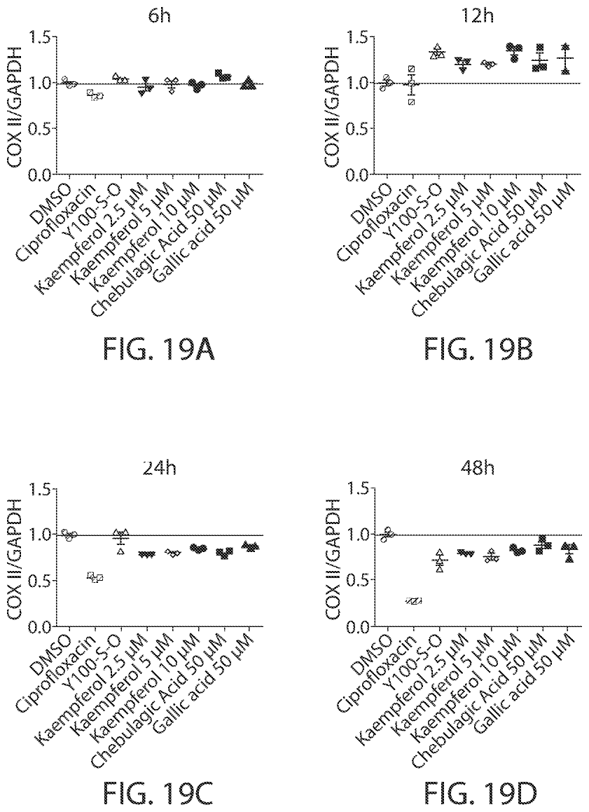

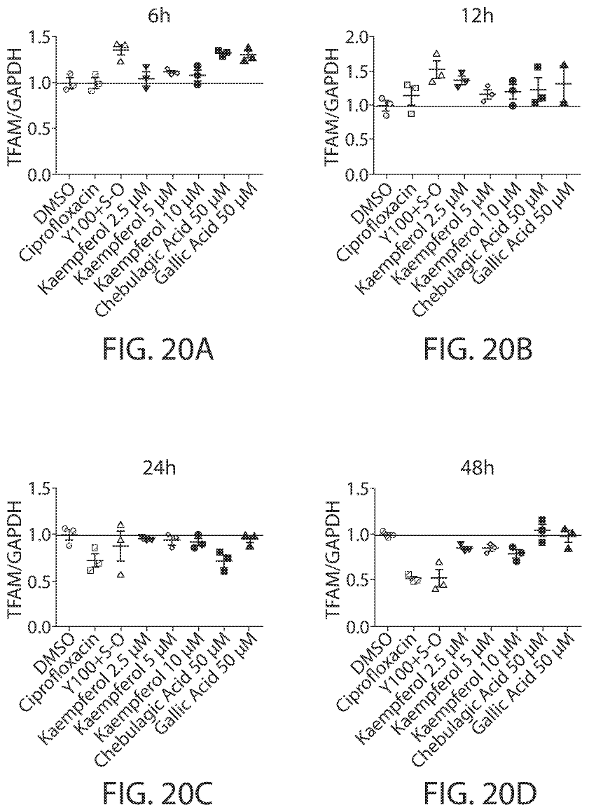

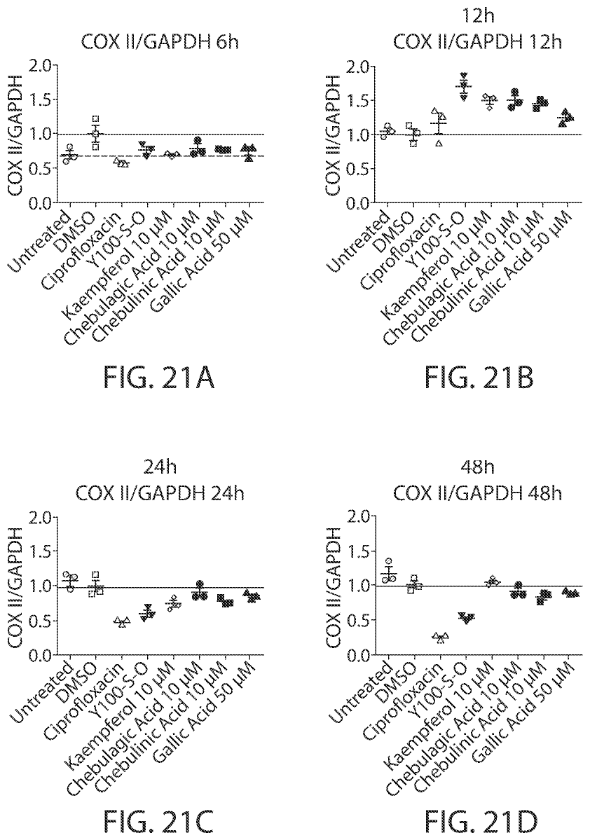

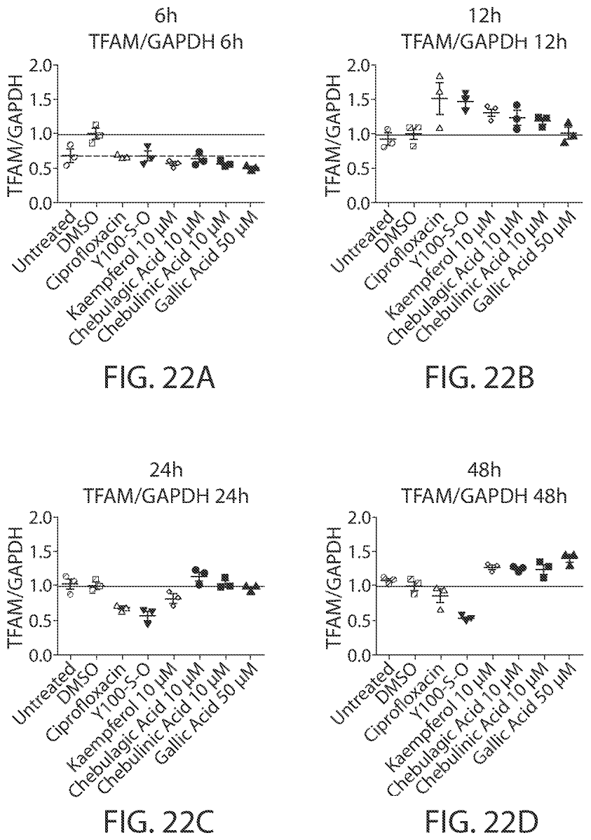

Selected constituents of Emblica extract were tested in vitro for induced expression of mitochondrial biogenesis regulatory proteins including COXII and TFAM at timepoints of 6, 12, 24, and 48 hours post-administration, generally as described in previous examples. Formulations of varying concentrations were tested. The results are presented in the graphs of FIGS. 10A through 22D. Briefly, chebulagic acid, chebulinic acid, kaempferol, ellagic acid, ascorbic acid, citric acid, gallic acid, quercetin, punicalagin were tested at concentrations ranging from 2.5 μM to 100 μM for induced expression of the target proteins.

### Example 9: Nanoencapsulation of Emblica Extract and Chebulinic Acid

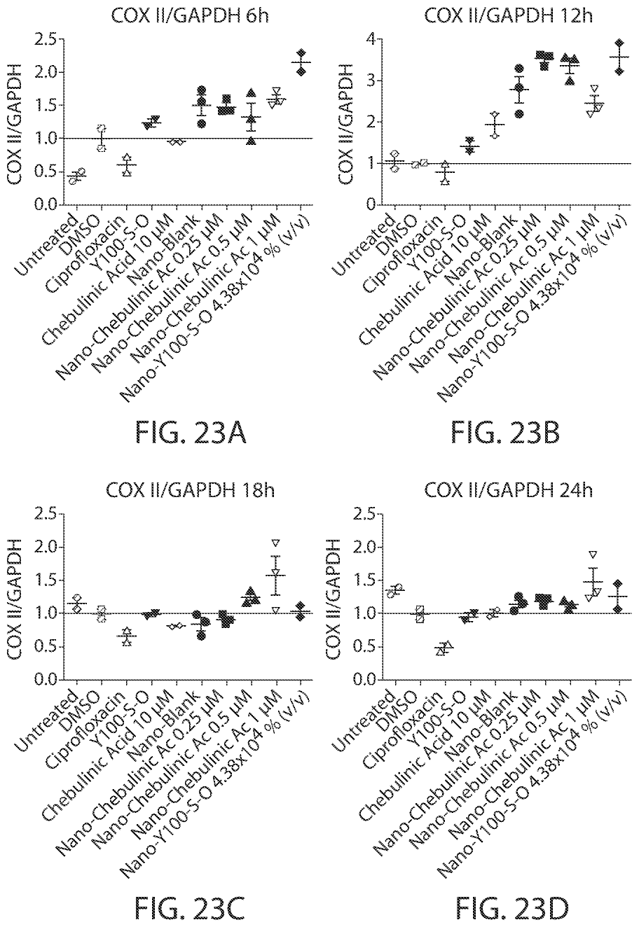

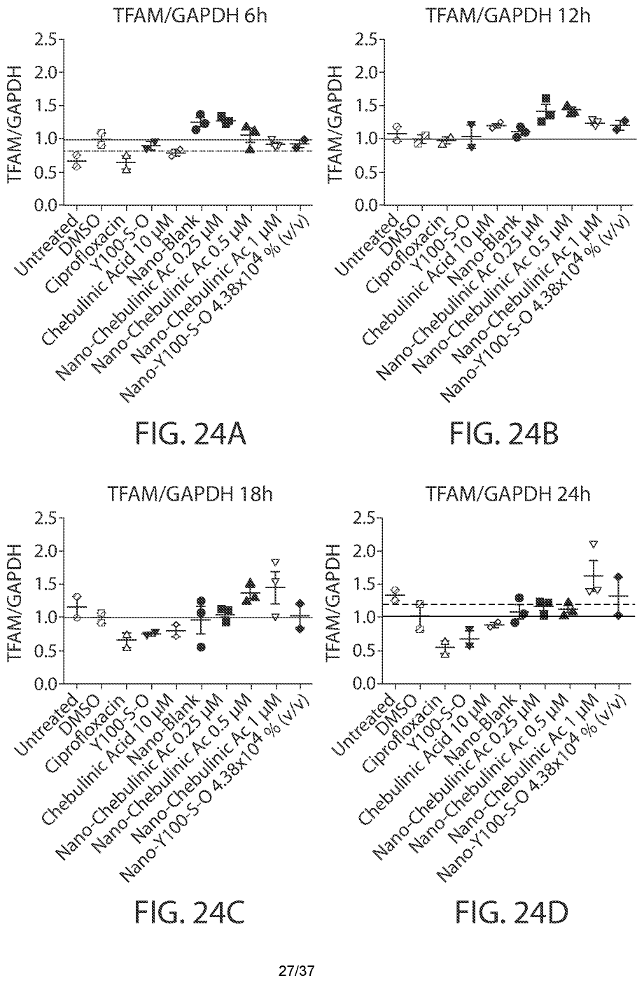

Emblica extract and chebulinic acid formulations and nanoencapsulated chebulinic acid formulations were tested in vitro for induced expression of mitochondrial biogenesis regulatory proteins COXII and TFAM at timepoints of 6, 12, 18, and 24 hours post-administration, generally as described in Example 4. Formulations of varying concentrations were tested. The results are presented in the graphs of FIGS. 23A-23D (COXII) and 24A-24D (TFAM).

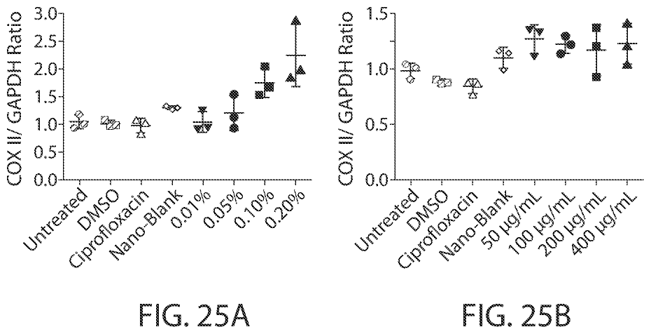

Induced expression of COXII at 6 hours post-administration of varying concentrations of nanoparticle loading from 0.01% to 0.2% Emblica extract and 50 to 400 μg/L was tested. The results are presented in the graphs of FIGS. 31A-31B. As shown in the graphs of FIGS. 25A-25B, the greater effect was observed as a result of greater compound loading.

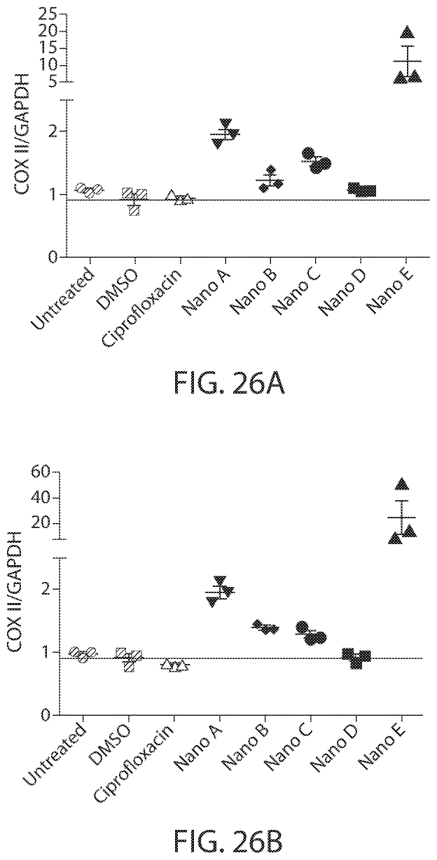

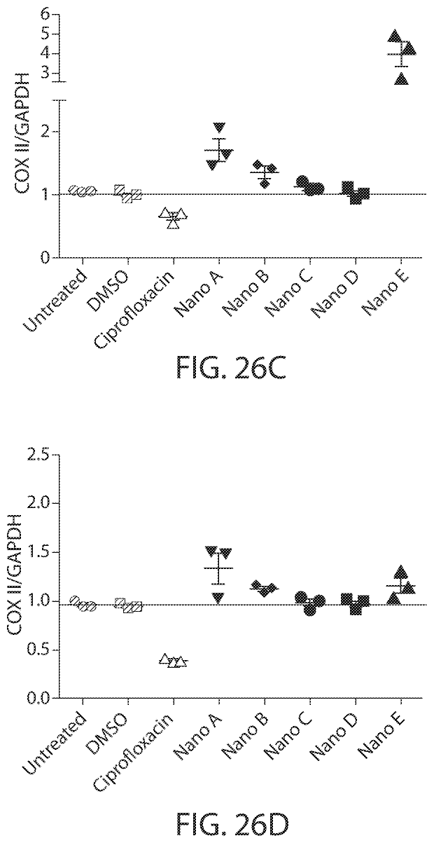

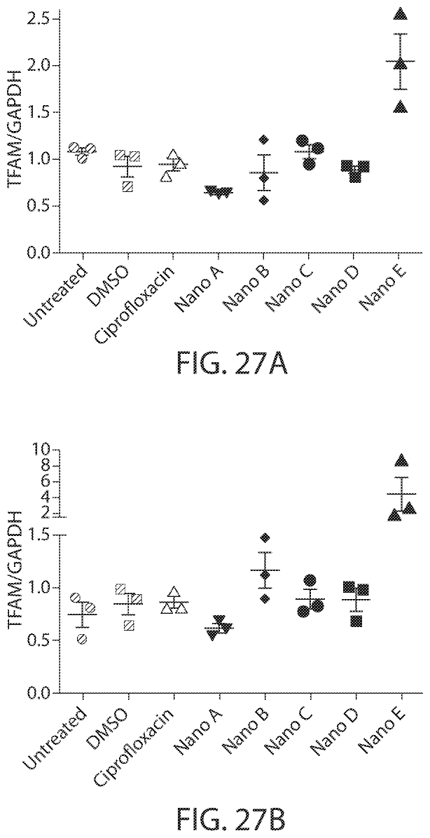

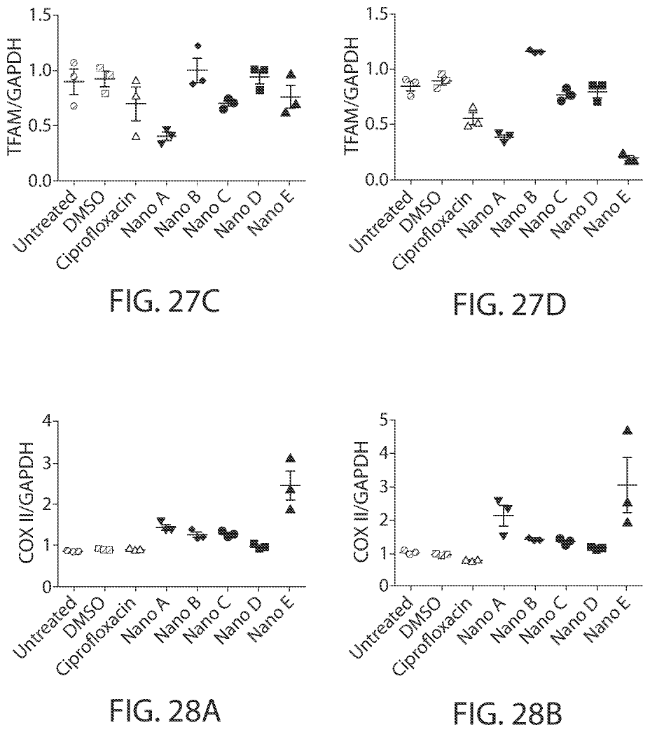

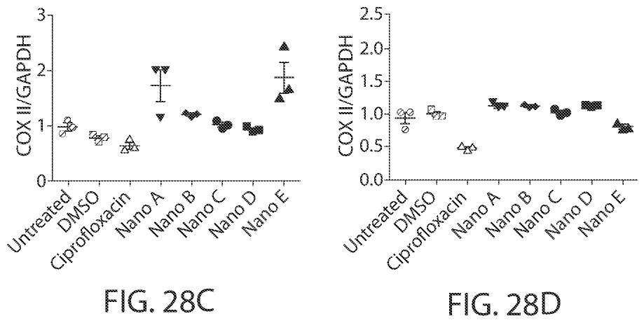

Induced expression of COXII and TFAM at 6, 12, 24, and 48 hours post-administration of nanoparticle compositions sized from 4.4 μm to 95 μm (with and without Emblica extract) was tested. The results are presented in the graphs of FIGS. 26A-26D (COXII), FIGS. 27A-27D (TFAM), and FIGS. 28A-28D (COXII). In the graphs, the x axis legend is as follows:
Nano A: Y100 (Emblica extract) powder loaded (40% LF; mean particle size=11.6 μm)
Nano B: Y100 (Emblica extract) oil loaded (42% LF; mean particle size=9.4 μm)
Nano C: undoped blank (mean particle size=7.7 μm)
Nano D: large size octyl-doped blank (mean particle size=95 μm)
Nano E; small size octyl-doped blank (mean particle size=4.4 μm)

As shown in the graphs of FIGS. 26A-26D, 27A-27D, and 28A-28D, the greater effect was generally observed as a result of smaller particles.

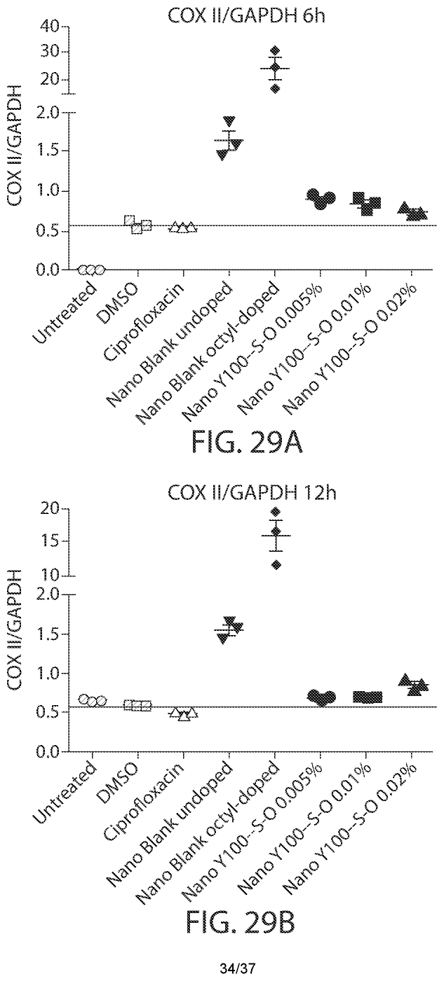

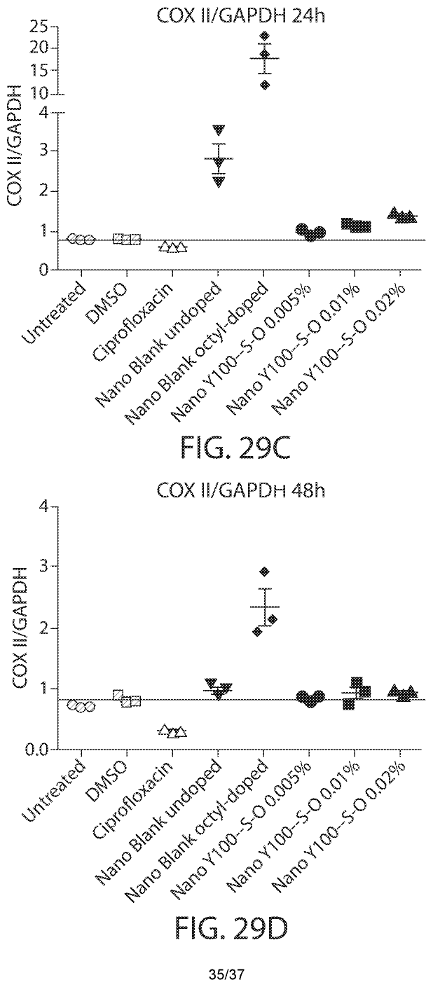

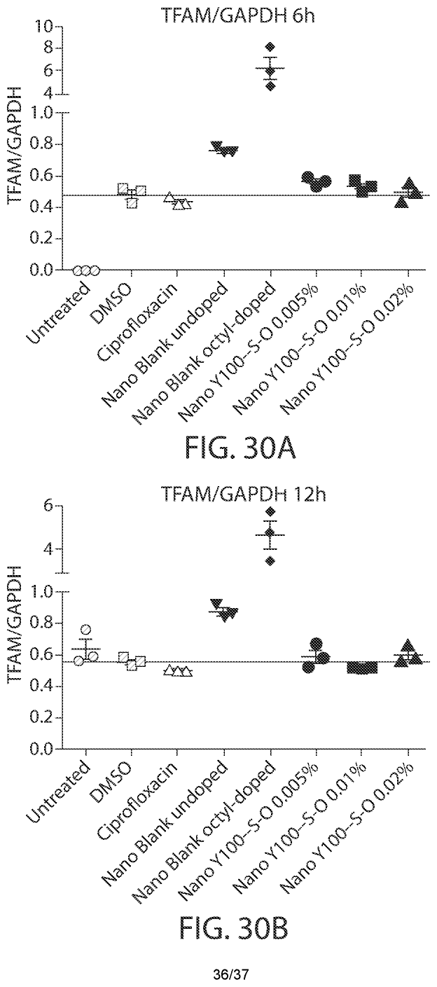

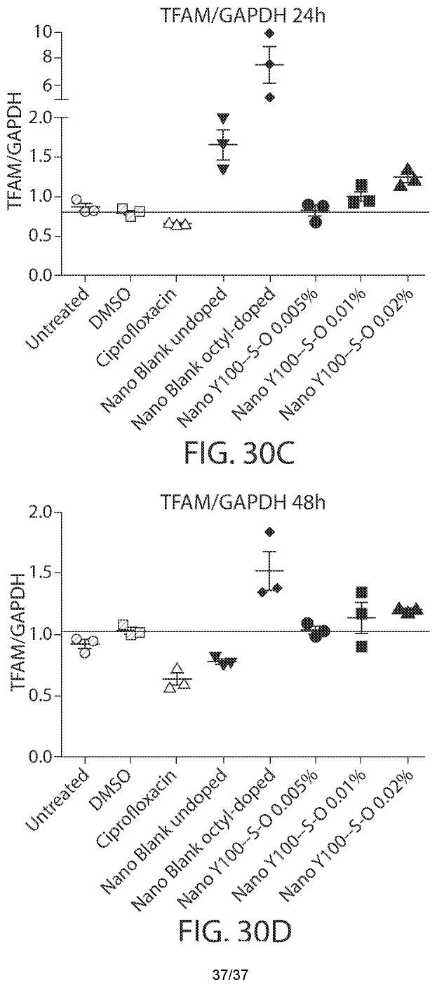

Induced expression of COXII and TFAM at 6, 12, 24, and 48 hours post-administration of nanoparticle compositions having a similar size (with and without Emblica extract) (with octyl-doping and without octyl-doping) was tested. The results are presented in the graphs of FIGS. 29A-29D (COXII), and FIGS. 30A-30D (TFAM). As shown in the data presented in FIGS. 29A-29D and 30A-30D, octyl-doped particles showed a greater expression than their undoped counterparts.

Overall, the data show that induction of mitochondrial biogenesis depends on nanoparticle load and the size of nanoparticles affects mitochondrial biogenesis. Differences in nanoparticle preparation (between octyl-doped and undoped) and load have an effect on mitochondrial biogenesis induction.

### Materials and Methods

Creation of mtDNA-Depleter Mice

D1135A-POLG1 site-directed mutation was created in the full-length human POLG1 complementary DNA (cDNA) using the site-directed mutagenesis kit (Agilent, Santa Clara, CA, USA). The primer sequences used for site-directed mutagenesis are as follows, with the mutated site in upper case: D1135A_F: 5′-gcatcagcatccatgCGgaggttcgctacctgg-3′ and D1135A_R: 5′-ccaggtagcgaacctcCGcatggatgctgatgc-3′. Mutations were confirmed by sequencing. D1135A-POLG1 cDNA was subcloned into the dox-inducible mammalian expression vector, pTRE-Tight-BI-AcGFP1 (Clontech, Palo Alto, CA, USA). To obtain germline transmission of human D1135A-POLG1 (POLG1-DN), microinjection of the pTRE-Tight-BI-AcGFP1-D1135A-POLG1 construct into fertilized oocytes from C57BL/6 mouse was carried out. Potential founders were identified by screening genomic DNA from tail biopsies for the presence of the human Polg1 transgene using the PCR. The heterozygous human POLG1-positive (+/POLG1-DN⁺) founder male mice were mated with CAG-rtTA3 (rtTA) C57BL/6 female mice (Jackson Laboratories, stock no. 016532) to obtain +/POLG1-DN⁺ rtTA⁺ heterozygous transgenic mice. The +/POLG1-DN⁺ rtTA⁺ heterozygous mice were intercrossed to generate homozygous POLG1-DN⁺ rtTA⁺/POLG1-DN⁺ rtTA⁺ mice (mtDNA-depleter mice). This cross resulted in normal litter size (6-7 pups) and Mendelian distributions of genotypes, that is, 1:2:1 distribution of wild-type, heterozygous +/POLG1-DN⁺ or +/rtTA⁺ and homozygous POLG1-DN⁺ rtTA⁺/POLG1-DN⁺ rtTA⁺ showing that homozygosity for POLG1-DN allele does not result in embryonic or postnatal lethality. All the mice were given dox in diet (200 mg/kg diet) and water (2 mg/ml dox in 5% sucrose water) ad libitum. All animal experiments were conducted by following guidelines established by the Institutional Animal Care and Use Committee.

### Histological and Immunohistochemical Analyses

Skin from the dorsal side as well as other tissues was fixed in buffered formalin, embedded in paraffin, sectioned (5 μM), and stained with hematoxylin and eosin. Skin sections were stained with Giemsa stain to detect mast cells, while MPO, CD3, CD163, and Pax-5 antibodies were used for detection of other types of inflammatory cells by immunohistochemical analyses (Carson, et al., Histotechnology: A Self-Instruction Text, 3 ed., American Society for Clinical Pathology Press, Hong Kong, 2009).

RT-PCR and mtDNA Content Analyses

To measure relative gene expression by RT-PCR, total cellular RNA from the skin samples was isolated using Trizol (Invitrogen, Carlsbad, CA, USA). Approximately, 1000-2000 ng RNA was normalized across samples, and cDNA was generated using the Iscript cDNA synthesis kit (Bio-Rad Laboratories, Hercules, CA, USA). cDNA was then subjected to RT-PCR using Green Taq PCR mixture (Promega, Madison, WI, USA) and gene-specific primers as given in Table 1 below. PCR products were run on 1.5 to 2% agarose gel and photographed using gel documentation system. At least three biological replicates were used in each PCR. β2-Microglobulin or RNU6B was used as an internal control in each PCR.

mtDNA content analyses in the skin and other tissues were carried out as reported earlier (Singh et al., PloS One, 10, e0139846, 2015). Briefly, the mtDNA content was analyzed by real-time PCR by absolute quantification with the following primers: mMitoF: 5′-CTAGAAACCCCGAAACCAAA-3′, mMitoR: 5′-CCAGCTATCACCAAGCTCGT-3′, mB2MF: 5′-ATGGGAAGCCGAACATACTG-3′, and mB2MR: 5′-CAGTCTCAGTGGGGGTGAAT-3′. Beta-2-Microglobulin (B2M) was used as an internal control.

TABLE 7

| | | |
|---|---|---|
| Target | Forward primer | Reverse primer |

Primers used for genotyping

| | | |
|---|---|---|
| POLG1 | CAA GGT CCA GAG AGA AAC TG | CTC TGT ACC ACC CAA TTC AC |
| CAG-rtTA | CTG CTG TCC ATT CCT TAT TC | CGA AAC TCT GGT TGA CAT G |
| GFP | GGG CAA TAA GAT GGA GTA CA | TGG ACA GGT AGT GGT TAT CG |

Primers used for RT-PCR

| | | |
|---|---|---|
| POLG1 | CCA GGG AGA GTT TAT AAC CA | CAA ATT CCT CAA ACA GCC AC |
| COXII | GGC ACC TTC ACC AAA ATC AC | CGG TTG TTG ATT AGG CGT TT |
| NDI | CCT ATC ACC CTT GCC ATC AT | TTG CTG CTT CAG TTG ATC GT |
| NF-κB | TGG CCG TGG AGT ACG ACA A | GCA TCA CCC TCC AGA AGC A |
| MMP2 | ACC TGA ACA CTT TCT ATG GCT | CTT CCG CAT GGT CTC GAT G |
| | G | |
| MMP9 | CTG GAC AGC CAG ACA CTA AAG | CTC GCG GCA AGT CTT CAG AG |
| TIMP1 | CTT GGT TCC CTG GCG TAC TC | ACC TGA TCC GTC CAC AAA |
| | | CAG |
| COL1A1 | CTG GCG GTT CAG GTC CAA T | TTC CAG GCA ATC CAC GAG C |
| Cyclooxygenase | AAC CGC ATT GCC TCT GAA T | CAT GTT CCA GGA GGA TGG AG |
| 2 | | |
| CCL5 | AGA TCT CTG CAG CTG CCC TCA | GGA GCA CTT GCT GCT GGT GTA |
| | | G |
| IL28a | AGG TCT GGG AGA ACA TGA CTG | CTG TGG CCT GAA GCT GTG TA |
| | | SEQ ID NO: 24 |
| IFNB1 | GTC ATG GGT TTC TCA TGA AGA | CAG ACC CCT TCC AGT GAT TCA |
| | ACA G | TC |
| | | SEQ ID NO: 26 |
| VEGF | GAG GAT GTC CTC ACT CGG ATG | GTC GTG TTT CTG GAA GTG AGC |
| | | AA |
| IGF1R | CGA GCT TCC TGT GAA AGT GAT | CAC GTT ATG ATG ATT CGG TTC |
| | GT | TTC |
| Klotho | GGA CAT TTC CCT GTG ACT TTG | AGA GAG AGT AGT GTC CAC |
| | C | TTG AAC GT |
| MRPS5 | AAC CAC TGT CTG ACC AGC TTG | AGT CTC TGC TAA TGC GCC TTT |
| RNU6B | CTC GCT TCG GCA GCA CA | AAC GCT TCA CGA ATT TGC GT |
| B2M | ATG GGA AGC CGA ACA TAC TG | CAG TCT CAG TGG GGG TGA AT |

### Microarray Gene Analysis

Total RNA samples were extracted from POLG1 D1135A expressing MCF-7 cells after 5 days of dox induction and from the cells grown in the absence of dox for 5 days by Trizol extraction method (Invitrogen). Illumina human microarray gene expression analysis was performed with total RNA samples as described earlier.

### BN-PAGE and Western Blot Analyses

Mitochondrial isolation was carried out as previously described (Johnstone et al., J Biol Chem, 277, 42197-42204, 2002). To analyze mitochondrial OXPHOS super complexes, Blue-Native polyacrylamide gel electrophoresis (BN-PAGE) was performed with mitochondrial fractions prepared from the skin samples as described previously (Schagger et al., Methods Enzymol, 260, 190-202, 1995). Protein expression of mitochondrial OXPHOS subunits in the skin samples was carried out following standard immunoblots. A premixed cocktail containing primary monoclonal antibodies (Mitosciences, Eugene, OR, USA) against subunits of OXPHOS complexes was used to detect OXPHOS super complexes in BN-PAGE analyses and protein expression of OXPHOS subunits in immunoblot analyses. Voltage-dependent anion channel (VDAC) or β-actin antibodies were used as loading controls.

### Analysis of Enzymatic Activities of OXPHOS Complexes

Isolated mitochondria were used for the measurement of enzymatic activities of OXPHOS complexes as previously described (Owens et al., PloS One, 6, e23846, 2011).

### Transmission Electron Microscopy

Transmission electron microscopic analyses of skin samples were carried as described previously (NAG et al., J Mol Cell Cardiol, 15, 301-317, 1983). Images were taken using the FEI-Tecnai electron microscope.

### Cell Culture

Skin fibroblasts from wild-type C57BL/6 (control cells) and mtDNA-depleter mice containing the D1135A-POLG1 site-directed mutation (POLG1-DN cells) were generated and spontaneously immortalized as described (Todaro et al., J Cell Biol, 17, 299-313 (1963). These cells were maintained in DMEM/F12 (Cellgro, Herndon, VA) supplemented with 10% FBS (Atlanta Biologicals, Lawrenceville, GA). To induce POLG1-DN expression in skin fibroblasts, 1 μg/ml dox dissolved in water was added to the cells in culture and after 6 days of incubation, cells were washed with PBS and collected in Trizol for isolation of total RNA.

To estimate cell proliferation and cell survival, MTT assays were carried out as described previously (Ronghe et al., J Steroid Biochem Mol Biol, 144 PtB, 500-512, 2014). Both control and POLG1-DN cells were first treated with dox (1 μg/ml) for 3 days and then cells were plated at a density of 3000 cells/well in 96 well plate with or without dox (1 μg/ml) containing culture media. Readings were taken at every 24 hours.

### Preparation of Emblica Extract

Emblica officinalis was obtained from commercially available caplets (Himalaya Drug Company, Sugar Land, TX). Each caplet contains 600 mg (250 mg fruit extract (45% tannins), 350 mg powder stem (2% tannins). Caplets were ground and dissolved in sterile water to make 100 mg/ml solution and then filtered. The Emblica solution was then mixed with an appropriate ointment base for topical application. Suitable ointment bases include, but are not limited to, dermabase ointment (MARCELLE®; water, mineral oil, propylene glycol, stearyl alcohol, cetyl esters, cetyl alcohol, glyceryl stearate, sodium lauryl sulfate, lecithin, and methylparaben) and 1:1 w/v) and Geritrex hydrophilic ointment (NDC 54162-670-14). The ointment base and Emblica solution may be added at any convenient ratio, for example from 1:5 w/v Emblica solution to ointment base to 5:1 w/v Emblica solution to ointment base. The ointment base and Emblica solution may be added at a 1:1 w/v ratio Emblica solution to ointment base.

### Preparation of Fucus Extract

Fucus vesiculosus (also known as bladder wrack, black tang, rockweed, bladder Fucus, sea oak, cut weed, dyers Fucus, red Fucus, and rock wrack) powder was obtained from Maine Coast Sea Vegetables, Inc. (Hancock, MN). An aqueous solution (100 mg/ml) solution was prepared from the Fucus powder and filtered. The Fucus solution was then mixed with an appropriate ointment base for topical application. Suitable ointment bases include, but are not limited to, dermabase ointment (MARCELLE®; water, mineral oil, propylene glycol, stearyl alcohol, cetyl esters, cetyl alcohol, glyceryl stearate, sodium lauryl sulfate, lecithin, and methylparaben) and 1:1 w/v) and Geritrex hydrophilic ointment (NDC 54162-670-14). The ointment base and Fucus solution may be added at any convenient ratio, for example from 1:5 w/v Fucus solution to ointment base to 5:1 w/v Fucus solution to ointment base. The ointment base and Fucus solution may be added at a 1:1 w/v ratio Fucus solution to ointment base.

### Emblica Extract In Vivo Experimental Design

For animal experiments, the dorsal skin of the mice was depilated (for example, shaved under low-dose isoflurane inhalation anesthesia) approximately 2 days before initiation of administration of a composition to the skin. Various active compositions and control compositions were applied topically to the dorsal skin daily as described. The skin of the mice was depilated prior to collection and analysis.

Emblica extract ointment at a concentration of 100 mg/mL aqueous solution was applied topically each day to a defined shaved area of the dorsal skin of the mice. Control compositions comprised the same amount of ointment base without addition of Emblica extract. Fucus extract ointment at a concentration of 100 mg/mL aqueous solution was applied topically each day to a defined shaved area of the dorsal skin of the mice. Control compositions comprised the same amount of ointment base without addition of Fucus extract.

mtDNA-depleter mice (containing the D1135A-POLG1 mutation) as well as wild-type C57BL/6 mice (control) were used in the prevention and therapeutic experiments.

In the preventive experiments, daily Emblica extract treatment as described above was started 7 days before starting the dox-mediated induction of POLG1-DN. Both mtDNA-depleter and wild-type C57BL/6 mice were divided in two treatment groups: i) a control group (treated with ointment base only, n=5); and ii) a test group (treated with Emblica extract ointment, n=5). Daily administration continued through the end of the experiment (up to 112 days).

In the therapeutic experiments, the wild-type C57BL/6 mice (control) and mtDNA-depleter mice were first induced with dox for 30 days and then Emblica extract treatment as described above was applied daily to the end of the experiment (up to 112 days) Both mtDNA-depleter and wild-type C57BL/6 mice were divided in two treatment groups: i) a control group (treated with ointment base only, n=5); and ii) a test group (treated with Emblica extract ointment, n=5).

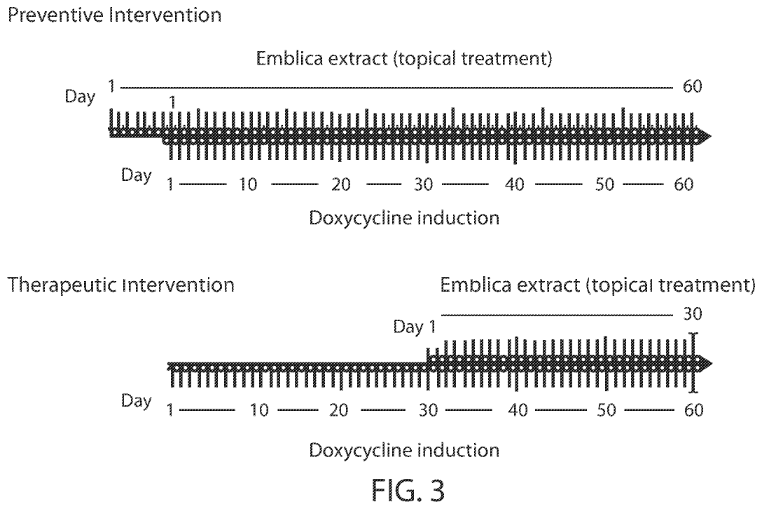

For periods of dox administration, mice were given dox in diet (200 mg/kg diet) and water (2 mg/ml dox in 5% sucrose water) ad libitum for Examples 1 to 13 and mice were given dox in diet (2 mg/kg diet) and water (2 mg/ml dox in 5% sucrose water) ad libitum for Examples 14 to 15. Schematics of both preventive and therapeutic in vivo experiments are shown in FIG. 3, noting that in certain experiments in Examples 3-5 the time course of Emblica extract or Fucus extract administration continued beyond the end of the time periods specified in FIG. 3 (i.e., up to 112 days).

### Statistical Analyses

Statistical analyses were performed using unpaired Student's t test. Data are expressed as mean±s.e.m. P values <0.05 were considered significant. All cellular experiments were repeated at least three times.

The phraseology and terminology used herein is for the purpose of description and should not be regarded as limiting. As used herein, the term “plurality” refers to two or more items or components. The terms “comprising.” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Having thus described several aspects of at least one embodiment, it is to be appreciated various alterations, modifications, and improvements will readily occur to those skilled in the art. Any feature described in any embodiment may be included in or substituted for any feature of any other embodiment. Such alterations, modifications, and improvements are intended to be part of this disclosure and are intended to be within the scope of the invention. Accordingly, the foregoing description and drawings are by way of example only.

Those skilled in the art should appreciate that the parameters and configurations described herein are exemplary and that actual parameters and/or configurations will depend on the specific application in which the disclosed methods and materials are used. Those skilled in the art should also recognize or be able to ascertain, using no more than routine experimentation, equivalents to the specific embodiments disclosed.

What is claimed is:

1. A method of enhancing or improving fertility in a subject, the method comprising administering to the subject a composition comprising an effective amount of one or more of an Emblica extract, a Fucus extract, and a chebula extract, or a pharmaceutically acceptable form thereof, or one or more compounds constituent of one or more of an Emblica extract, a Fucus extract, and a chebula extract, or a pharmaceutically acceptable form thereof, or one or more compounds having a similarity score of at least 95% with a compound constituent of one or more of an Emblica extract, a Fucus extract, and a chebula extract, or a pharmaceutically acceptable form thereof.

2. A method of extending reproductive longevity in a subject, the method comprising administering to the subject a composition comprising an effective amount of one or more of an Emblica extract, a Fucus extract, and a chebula extract, or a pharmaceutically acceptable form thereof, or one or more compounds constituent of one or more of an Emblica extract, a Fucus extract, and a chebula extract or a pharmaceutically acceptable form thereof, or one or more compounds having a similarity score of at least 95% with a compound constituent of one or more of an Emblica extract, a Fucus extract, and a chebula extract, or a pharmaceutically acceptable form thereof.

3. A method of increasing oocyte mitochondrial mass or preventing a decrease of oocyte mitochondrial mass in a subject, the method comprising administering to the subject a composition comprising an effective amount of one or more of an Emblica extract, a Fucus extract, and a chebula extract, or a pharmaceutically acceptable form thereof, or one or more compounds constituent of one or more of an Emblica extract, a Fucus extract, and a chebula extract, or a pharmaceutically acceptable form thereof, or one or more compounds having a similarity score of at least 95% with a compound constituent of one or more of an Emblica extract, a Fucus extract, and a chebula extract, or a pharmaceutically acceptable form thereof.

4. A method of improving embryo development or improving embryo fertilization rate, the method comprising administering to a subject source of the embryo or to the embryo a composition comprising an effective amount of one or more of an Emblica extract, a Fucus extract, and a chebula extract, or a pharmaceutically acceptable form thereof, or one or more compounds constituent of one or more of an Emblica extract, a Fucus extract, and a chebula extract or a pharmaceutically acceptable form thereof, or one or more compounds having a similarity score of at least 95% with a compound constituent of one or more of an Emblica extract, a Fucus extract, and a chebula extract, or a pharmaceutically acceptable form thereof.

5. A method of treating or preventing an ovarian disease or condition in a subject, the method comprising administering to the subject a composition comprising an effective amount of one or more of an Emblica extract, a Fucus extract, and a chebula extract, or a pharmaceutically acceptable form thereof, or one or more compounds constituent of one or more of an Emblica extract, a Fucus extract, and a chebula extract, or a pharmaceutically acceptable form thereof, or one or more compounds having a similarity score of at least 95% with a compound constituent of one or more of an Emblica extract, a Fucus extract, and a chebula extract, or a pharmaceutically acceptable form thereof.

6. A method of improving embryo development or improving embryo fertilization rate, the method comprising transferring ooplasm from a donor oocyte to a recipient oocyte to be fertilized to form the embryo, the donor oocyte having a mitochondrial DNA (mtDNA) content greater than the recipient oocyte, wherein at least one of a donor subject, a recipient subject, and the embryo was administered a composition comprising an effective amount of one or more of an Emblica extract, a Fucus extract, and a chebula extract, or a pharmaceutically acceptable form thereof, or one or more compounds constituent of one or both of an Emblica extract, a Fucus extract, and a chebula extract, or a pharmaceutically acceptable form thereof, or one or more compounds having a similarity score of at least 95% with a compound constituent of one or more of an Emblica extract, a Fucus extract, and a chebula extract, or a pharmaceutically acceptable form thereof.

7. A method of reducing, preventing, or delaying perimenopause, menopause, or a symptom thereof in a subject, the method comprising administering to the subject a composition comprising an effective amount of one or more of an Emblica extract, a Fucus extract, and a chebula extract, or a pharmaceutically acceptable form thereof, or one or more compounds constituent of one or more of an Emblica extract, a Fucus extract, and a chebula extract, or a pharmaceutically acceptable form thereof, or one or more compounds having a similarity score of at least 95% with a compound constituent of one or more of an Emblica extract, a Fucus extract, and a chebula extract, or a pharmaceutically acceptable form thereof.

8. The method of any of the preceding claims, comprising administering to the subject an effective amount of an Emblica extract, one or more compound constituent of an Emblica extract or having a similarity score of at least 95% with a compound constituent of an Emblica extract, or a pharmaceutically acceptable form thereof.

9. The method of any of the preceding claims, comprising administering to the subject an effective amount of a Fucus extract, one or more compound constituent of a Fucus extract or having a similarity score of at least 95% with a compound constituent of a Fucus extract, or a pharmaceutically acceptable form thereof.

10. The method of any of the preceding claims, comprising administering to the subject an effective amount of a chebula extract, one or more compound constituent of a chebula extract or having a similarity score of at least 95% with a compound constituent of a chebula extract, or a pharmaceutically acceptable form thereof.

11. The method of any of the preceding claims, wherein the Emblica extract is derived from Emblica officinalis.

12. The method of any of the preceding claims, wherein the compound constituent of the Emblica extract is a benzoic acid substituted with 1 to 5 hydroxy groups and optionally 1 to 3 O—(C1-C5 alkyl) or O—(C1-C5 alkenyl) groups, or a pharmaceutically acceptable form thereof, or a benzene substituted with —CH═CH—(CH2)a-C(O)OH, wherein a is 0 to 5, and 1 to 5 hydroxy groups, or a pharmaceutically acceptable form thereof, or a combination of the foregoing.

13. The method of any of the preceding claims, wherein the compound constituent of the Emblica extract is gallic acid, vanillic acid, chlorogenic acid, 5 caffeic acid, syringic acid, coumaric acid, quercetin, Emblicanin A, Emblicanin B, punigluconin, pedunculagin, punicafolin, phyllanemblin, kaempferol, ellagic acid, chebulinic acid, chebulagic acid, punicalagin, a metabolite of any of the foregoing, a compound having a similarity score of at least 95% with any of the foregoing, or a pharmaceutically acceptable form of any of the foregoing.

14. The method of any of the preceding claims, wherein the Fucus extract is derived from Fucus vesiculosus, Fucus serratus, Fucus, spiralis, or Fucus guiryi.

15. The method of any of the preceding claims, wherein the compound constituent of the Fucus extract is a benzoic acid substituted with 1 to 5 hydroxy groups and optionally 1 to 3 O—(C1-C5 alkyl) or O—(C1-C5 alkenyl) groups, or a pharmaceutically acceptable form thereof, or a benzene substituted with —CH═CH—(CH₂)a-C(O)OH, wherein a is 0 to 5, and 1 to 5 hydroxy groups, or a pharmaceutically acceptable form thereof, or a combination of the foregoing.

16. The method of any of the preceding claims, wherein the compound constituent of the Fucus extract is gallic acid, vanillic acid, chlorogenic acid, caffeic acid, syringic acid, coumaric acid, quercetin, fucoidan, punigluconin, pedunculagin, punicafolin, phyllanemblin, kaempferol, ellagic acid, chebulinic acid, chebulagic acid, punicalagin, a metabolite of any of the foregoing, a compound having a similarity score of at least 95% with any of the foregoing, or a pharmaceutically acceptable form of any of the foregoing.

17. The method of any of the preceding claims, wherein the chebula extract is derived from Terminilia chebula, Terminalia arborea, or Lumnitzera racemose.

18. The method of any of the preceding claims, wherein the compound constituent of the chebula extract is a benzoic acid substituted with 1 to 5 hydroxy groups and optionally 1 to 3 O—(C1-C5 alkyl) or O—(C1-C5 alkenyl) groups, or a pharmaceutically acceptable form thereof, or a benzene substituted with —CH═CH—(CH₂)a-C(O)OH, wherein a is 0 to 5, and 1 to 5 hydroxy groups, or a pharmaceutically acceptable form thereof, or a combination of the foregoing.

19. The method of any of the preceding claims, wherein the compound constituent of the chebula extract is gallic acid, vanillic acid, chlorogenic acid, caffeic acid, syringic acid, coumaric acid, quercetin, fucoidan, punigluconin, pedunculagin, punicafolin, phyllanemblin, kaempferol, ellagic acid, chebulinic acid, chebulagic acid, punicalagin, a metabolite of any of the foregoing, a compound having a similarity score of at least 95% with any of the foregoing, or a pharmaceutically acceptable form of any of the foregoing.

20. The method of any of the preceding claims, wherein the composition comprises two or more of: an effective amount of an Emblica extract, or a pharmaceutically acceptable form thereof, or a compound constituent of an Emblica extract, or a pharmaceutically acceptable form thereof, or a compound having a similarity score of at least 95% with a compound constituent of an Emblica extract, or a pharmaceutically acceptable form thereof; an effective amount of a Fucus extract, or a pharmaceutically acceptable form thereof, or a compound constituent of a Fucus extract, or a pharmaceutically acceptable form thereof, or a compound having a similarity score of at least 95% with a compound constituent of a Fucus extract, or a pharmaceutically acceptable form thereof; and an effective amount of a chebula extract, or a pharmaceutically acceptable form thereof, or a compound constituent of a chebula extract, or a pharmaceutically acceptable form thereof, or a compound having a similarity score of at least 95% with a compound constituent of a chebula extract, or a pharmaceutically acceptable form thereof.

21. The method of any of the preceding claims, wherein the composition is fortified with one or more compounds constituent of an Emblica extract, or a pharmaceutically acceptable form thereof, or a compound having a similarity score of at least 95% with a compound constituent of an Emblica extract, or a pharmaceutically acceptable form thereof.

22. The method of any of the preceding claims, wherein the composition is fortified with one or more compounds constituent of a Fucus extract, or a pharmaceutically acceptable form thereof, or a compound having a similarity score of at least 95% with a compound constituent of a Fucus extract, or a pharmaceutically acceptable form thereof.

23. The method of any of the preceding claims, wherein the composition is fortified with one or more compounds constituent of a chebula extract, or a pharmaceutically acceptable form thereof, or a compound having a similarity score of at least 95% with a compound constituent of a chebula extract, or a pharmaceutically acceptable form thereof.

24. The method of any of the preceding claims, wherein the one or more compounds constituent of the Emblica extract or having a similarity score of at least 95% with a compound constituent of the Emblica extract is purified, e.g., at least 80% purified, at least 85% purified, at least 90% purified, at least 95% purified, at least 98% purified, at least 99% purified, at least 99.9% purified, at least 99.99% purified, or at least 99.999% purified.

25. The method of any of the preceding claims, wherein the one or more compounds constituent of the Fucus extract or having a similarity score of at least 95% with a compound constituent of the Fucus extract is purified, e.g., at least 80% purified, at least 85% purified, at least 90% purified, at least 95% purified, at least 98% purified, at least 99% purified, at least 99.9% purified, at least 99.99% purified, or at least 99.999% purified.

26. The method of any of the preceding claims, wherein the one or more compounds constituent of the chebula extract or having a similarity score of at least 95% with a compound constituent of the chebula extract is purified, e.g., at least 80% purified, at least 85% purified, at least 90% purified, at least 95% purified, at least 98% purified, at least 99% purified, at least 99.9% purified, at least 99.99% purified, or at least 99.999% purified.

27. The method of any of the preceding claims, wherein the effective amount is a therapeutically effective amount.

28. The method of any of the preceding claims, wherein administration induces mitochondrial biogenesis and/or improves mitochondrial function.

29. The method of any of the preceding claims, wherein the effective amount or therapeutically effective amount is sufficient to induce mitochondrial biogenesis.

30. The method of any of the preceding claims, wherein administration increases expression of at least one protein selected from PGC-1a, TFAM, NRF-1, and COX II.

31. The method of any of the preceding claims, wherein administration modulates menstrual cycles, e.g., normalizes menstrual cycles, increases ovulation events, and/or increases an anti-Mullerian hormone (AMH) level of the subject or recipient subject.

32. The method of any of the preceding claims, wherein administration decreases the probability of embryonic aneuploidy and/or Leigh's Syndrome.

33. The method of any of the preceding claims, wherein administration reduces or lessens severity or frequency of at least one symptom of an ovarian diseases or conditions e.g., skin or vaginal dryness, pelvic pain or cramping, inflammation, prolonged or irregular menstrual cycles, and decreased ovulation events.

34. The method of any of the preceding claims, wherein the composition is administered topically.

35. The method of any of the preceding claims, wherein the composition is administered parenterally, e.g., intravenously, intraperitoneally, or intramuscularly.

36. The method of any of the preceding claims, wherein the composition is administered enterally.

37. The method of any of the preceding claims, wherein the composition is formulated as a topical solution, oil, cream, emulsion, or gel.

38. The method of any of the preceding claims, wherein the composition is formulated as a shampoo, conditioner, spray, cream, gel, balm, body wash, soap, lotion, or make-up.

39. The method of any of the preceding claim, wherein the composition is formulated as a parenteral liquid solution.

40. The method of any of the preceding claims, wherein the composition is formulated as an enteral capsule or tablet, or dietary supplement or food, e.g., food, food supplement, medical food, food additive, nutraceutical, or drink.

41. The method of any of the preceding claims, wherein the composition is administered locally.

42. The method of any of the preceding claims, wherein the composition is administered systemically.

43. The method of any of the preceding claims, wherein the composition is formulated for immediate release.

44. The method of any of the preceding claims, wherein the composition is formulated for extended release, e.g., controlled or sustained release.

45. The method of any of the preceding claims, wherein the composition is administered in combination with a standard of care treatment for improving fertility.

46. The method of any of the preceding claims, wherein the composition is administered in combination with a standard of care treatment for treating an ovarian disease or condition or a symptom thereof.

47. The method of any of the preceding claims, wherein the composition is administered in combination with one or more of clomiphene, tamoxifen, letrozole, metformin, gonadotrophins, gonadotrophin-releasing hormone, dopamine agonists, and other hormonal treatments.

48. The method of any of the preceding claims, wherein the composition is administered in combination with a surgical procedure, e.g., fallopian tube surgery, laparoscopic surgery to remove or destroy cysts or submucosal fibroids, or laparoscopic ovarian drilling.

49. The method of any of the preceding claims, wherein the composition is administered in combination with a UV-blocking agent, moisturizer, sunscreen, wrinkle cream, retinoid, alpha-hydroxy acid, beta-hydroxy acid, squalene, antioxidant, tretinoin, glycosaminoglycan (GAG), lactic acid, malic acid, citric acid, tartaric acid, hydroquinone, kojic acid, L-ascorbic acid, licorice extract, N-acetylglucosamine, niacinamide, soy, dermal filler or injection, e.g. hyaluronic acid or calcium hydroxylapatite, botulinum toxin, laser resurfacing procedure, ultrasound therapy, chemical peel, e.g. glycolic acid peel, trichloroacetic acid or salicylic acid, or dermabrasion procedure.

50. The method of any of the preceding claims, wherein the composition is administered in combination with a biomaterial, or encapsulated in a biomaterial, selected from an extracellular vesicle, collagen, hyaluronic acid, a synthetic biomaterial (e.g., polylactic acid (PLA), polyglycolic acid (PGA), polycaprolactone (PCL), polyethylene glycol (PEG)), fibrin, alginate, and a composite biomaterial.

51. The method of any of the preceding claims, wherein the ovarian disease or condition is selected from perimenopause, menopause, endometriosis, ovarian cysts, pre-menopausal or post-menopausal ovarian cancer, e.g., ovarian epithelial cancer, ovarian tumors, e.g., ovarian germ cell tumor, ovarian low malignant potential tumors, and ovarian stromal tumors, polycystic ovary syndrome (PCOS), primary ovarian insufficiency (POI), and ovarian torsion.

52. The method of any of the preceding claims, wherein the composition is administered in combination with one or more of hormone therapy, e.g., estrogen, progesterone, testosterone, or a synthetic form thereof, selective serotonin reuptake inhibitors (SSRIs), gabapentin, clonidine, hormonal contraceptives, gonadotropin-releasing hormone (GnRH) agonists or antagonists, heat therapy, pain relievers, nonsteroidal anti-inflammatory drugs (NSAID), such as ibuprofen or other analgesics, spironolactone, eflornithine, electrolysis, chemotherapy, radiation therapy, and surgery, e.g., hysterectomy, bilateral salpingo-oophorectomy, and debulking surgery, and lifestyle changes, such as weight loss, improved nutrition, and increased physical activity.

53. The method of any of the preceding claims, wherein the embryo is produced by in vitro fertilization (IVF).

54. The method of any of the preceding claims, further comprising measuring mitochondrial content of the embryo and selecting the embryo responsive to the measurement of mitochondrial content being within a predetermined range.

55. The method of any of the preceding claims, wherein the donor oocyte is autologous.

56. The method of any of the preceding claims, wherein the donor oocyte is allogeneic.

57. The method of any of the preceding claims, wherein the donor oocyte is xenogeneic.

58. The method of any of the preceding claims, wherein the ooplasm transfer is complete.

59. The method of any of the preceding claims, wherein the ooplasm transfer is partial, e.g., by electrofusion or direct ooplasmic injection.

60. The method of any of the preceding claims, wherein the transfer is performed by modified intracytoplasmic sperm injection (ICSI).

61. The method of any of the preceding claims, wherein the transfer is performed by autonomous germline mitochondrial energy transfer (AUGMENT) on the oocyte.

62. The method of any of the preceding claims, further comprising using CRISPR/Cas 9 gene editing technology on the mtDNA of the donor oocyte or the recipient oocyte.

63. The method of any of the preceding claims, further comprising using CRISPR/Cas 9 gene editing technology on mitochondrial DNA (mtDNA) of an oocyte of the subject to be fertilized to form the embryo.

64. The method of any of the preceding claims, further comprising introducing stem cell generated mitochondrial DNA (mtDNA) into an oocyte of the subject to be fertilized to form the embryo.

65. The method of any of the preceding claims, further comprising introducing antioxidants to a culture media of the embryo during development.

66. The method of any of the preceding claims, wherein the transfer is performed by nuclear genome transfer, e.g., oocyte spindle transfer, germinal vesicle (GV) transfer, pronuclear transfer (PNT), or polar body nuclear transfer (PBNT).

67. The method of any of the preceding claims, wherein the subject or recipient subject is characterized by age-related reduced fertility, infertility, and/or ovarian aging.

68. The method of any of the preceding claims, wherein the subject or recipient subject is 40 years or older.

69. The method of any of the preceding claims, wherein the subject or recipient subject is characterized by premature reproductive aging, e.g., primary ovarian insufficiency (POI), early onset prolonged or irregular menstrual cycles, early onset decreased ovulation events, premature menopause or perimenopause, and/or ovarian inflammation associated with premature reproductive aging.

70. The method of any of the preceding claims, wherein the subject or recipient subject is younger than 40 years.

71. The method of any of the preceding claims, wherein the subject or recipient subject is pre-menopausal.

72. The method of any of the preceding claims, wherein the subject or recipient subject is perimenopausal.

73. The method of any of the preceding claims, wherein the subject or recipient subject is post-menopausal.

74. The method of any of the preceding claims, wherein the subject or recipient subject has a diminished ovarian reserve (DOR).

75. The method of any of the preceding claims, wherein the subject or recipient subject has a normal ovarian reserve (NOR).

76. The method of any of the preceding claims, wherein the subject or recipient subject suffers from one or more of obesity, diabetes, chronic inflammation, high blood sugar, an autoimmune disease, and/or poor lifestyle factors, such as smoking, alcohol use, drug use, exposure to chemicals (e.g., bisphenol A, advanced glycation end products (AGE)) and/or radiation, poor nutrition, e.g., increased ingestion of charred foods, increased ingestion of saturated fats, and/or reduced ingestion of fruits and vegetables, and/or reduced exercise.

77. The method of any of the preceding claims, wherein the subject or recipient subject is characterized by a reduced anti-Mullerian hormone (AMH) level, e.g., less than about 80 ng/ml, an increased follicle stimulating hormone (FHL) level, e.g., greater than about 10 IU/L, an increased estradiol level, and/or a reduced antral follicle count (AFC), e.g., less than about 5-7 total follicles.

78. The method of any of the preceding claims, wherein the subject or recipient subject is undergoing or has attempted in vitro fertilization (IVF).

79. The method of any of the preceding claims, wherein the subject or recipient subject is a poor responder to IVF.

80. The method of any of the preceding claims, wherein the subject or recipient subject is a normal responder to IVF.

81. The method of any of the preceding claims, wherein the subject or recipient subject suffers from reduced production of reproductive hormones, e.g., estrogen and/or progesterone, increased production of follicle-stimulating hormone (FSH), prolonged or irregular menstrual cycles, uterine or vaginal atrophy, e.g., decreased endometrial glands, diffuse glandular breakdown (apoptotic bodies) of the uterus, acute inflammation of the uterus, and/or decreased vaginal epithelial thickness, decreased ovulation events, ovarian inflammation, and/or infertility.

82. The method of any of the preceding claims, wherein the subject or recipient subject is characterized by a mitofusin (Mfn1) gene abnormality, a dynamic related protein 1 (Drp1) gene abnormality, a caseinolytic peptidase P (Clpp) gene abnormality, a growth differentiation factor 9 (GDF9) gene abnormality, a fragile-X mental retardation 1 (FMR1) gene abnormality, a transcription factor A (TFAM) gene abnormality, a mitochondrial DNA polymerase gamma (PolgA) gene abnormality, a mitochondrial inner membrane peptidase (IMP) gene abnormality, and/or a mitochondrial ABC transporter (MDR-1) gene abnormality.

83. The method of any of the preceding claims, wherein the subject or recipient subject is characterized by decreased gene expression of PPARy, PGC-1a, PGC-1b, ERR, NRF-1, NRF-2, SIRT1, SIRT3, SIRT4, and/or SIRT5.

84. The method of any of the preceding claims, wherein the subject or recipient subject is characterized by decreased AMP-activated protein kinase (AMPK) and or PTEN-induced kinase 1 (PINK1) protein expression and/or increased mammalian target of rapamycin (mTOR) protein expression.

85. The method of any of the preceding claims, wherein the subject or recipient subject has a somatic mitochondrial DNA mutation in an oocyte to be fertilized to form the embryo or the recipient oocyte, e.g., a T414G transverse mutation.

86. The method of any of the preceding claims, wherein the subject or recipient subject has an inherited mitochondrial DNA (mtDNA) mutation in an oocyte to be fertilized to form the embryo or the recipient oocyte, e.g., a T414G transverse mutation.

87. The method of any of the preceding claims, wherein the subject or recipient subject is characterized by decreased levels of mitochondrial DNA (mtDNA) in an oocyte to be fertilized to form the embryo or the recipient oocyte and/or reduced oocyte quality, e.g., chromosomal misalignment and/or spindle malformation.

88. The method of any of the preceding claims, wherein the subject or recipient subject is characterized by decreased gene expression of superoxide dismutase (SOD) and/or increased levels of Aldh3A2 enzyme.

89. The method of any of the preceding claims, wherein the subject or recipient subject is characterized by decreased mRNA expression of Prdx3, Prdx4, and/or Txn2 enzyme.

90. The method of any of the preceding claims, wherein the subject or recipient subject suffers from Turner's Syndrome, galactosemia, and/or Fragile X Syndrome.

91. The method of any of the preceding claims, wherein the subject or recipient subject is characterized by progressive external ophthalmoplegia (POE), spinocerebellar ataxia with epilepsy (SCAE), and/or mitochondrial encephalomyopathy, lactic acidosis, and stroke-like episodes (MELAS).

92. The method of any of the preceding claims, wherein the subject is characterized by a neurologic condition.

93. The method of any of the preceding claims, wherein the subject or recipient subject is characterized by dysfunctional folliculogenesis, e.g., follicle depletion or follicle dysfunction.

94. The method of any of the preceding claims, wherein the subject or recipient subject has decreased tertiary follicle counts, e.g., early antral, antral, and/or pre-ovulatory, and/or decreased corpora lutea (CL) follicle counts.

95. The method of any of the preceding claims, wherein the subject or recipient subject is characterized by down-regulation of estrogen receptors (ER) α and β in the ovary, reduced ESR1 or ESR2 gene expression, increased miR-206-3p RNA expression, reduced ovarian production of 17β-estradiol (E2), and/or increased lipid-laden ovarian stromal cells.

96. The method of any of the preceding claims, wherein the composition comprises a nanoparticle-based delivery carrier.

97. The method of any of the preceding claims, wherein the composition comprises a skin penetration enhancer or is administered in combination with a skin penetration enhancer, e.g., a chemical skin penetration enhancer or a physical skin penetration enhancer.

98. The method of any of the preceding claims, wherein the composition comprises a mitochondria-targeting agent or a delivery carrier functionalized with a mitochondria-targeting agent.

99. The method of any of the preceding claims, wherein the compound constituent of an Emblica extract was derived from, purified from, or isolated from the Emblica extract.

100. The method of any of the preceding claims, wherein the compound constituent of an Emblica extract was derived from, purified from, or isolated from a source other than the Emblica extract.

101. The method of any of the preceding claims, wherein the compound constituent of an Emblica extract was synthesized.

102. The method of any of the preceding claims, wherein the compound constituent of a Fucus extract was derived from, purified from, or isolated from the Fucus extract.

103. The method of any of the preceding claims, wherein the compound constituent of a Fucus extract was derived from, purified from, or isolated from a source other than the Fucus extract.

104. The method of any of the preceding claims, wherein the compound constituent of a Fucus extract was synthesized.

105. The method of any of the preceding claims, wherein the compound constituent of a chebula extract was derived from, purified from, or isolated from the chebula extract.

106. The method of any of the preceding claims, wherein the compound constituent of a chebula extract was derived from, purified from, or isolated from a source other than the chebula extract.

107. The method of any of the preceding claims, wherein the compound constituent of a chebula extract was synthesized.

108. A kit comprising a preparation comprising an effective amount of donor mtDNA in a therapeutically acceptable carrier and a composition comprising an effective amount of one or more of an Emblica extract, a Fucus extract, and a chebula extract, or a pharmaceutically acceptable form thereof, or one or more compounds constituent of one or more of an Emblica extract, a Fucus extract, and a chebula extract, or a pharmaceutically acceptable form thereof, or one or more compounds having a similarity score of at least 95% with a compound constituent of one or more of an Emblica extract, a Fucus extract, and a chebula extract, or a pharmaceutically acceptable form thereof.
